(19) United States
(12) Patent Application Publication
(10) Pub. No.: US 2009/0175846 A1
(43) Pub. Date: Jul. 9, 2009

(54) Oligodendrocyte-Myelin Glycoprotein Compositions and Methods of Use Thereof

(75) Inventors: Sha Mi, Belmont, MA (US); R. Blake Pepinsky, Arlington, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: BIOGEN IDEC MA INC, Cambridge, MA (US)

(21) Appl. No.: 12/091,564

(22) PCT Filed: Oct. 27, 2006
(86) PCT No.: PCT/US06/41966

§ 371 Date: Oct. 14, 2008

Related U.S. Application Data
(60) Provisional application No. 60/730,357, filed on Oct. 27, 2005.
(60) Provisional application No. 60/735,170, filed on Nov. 10, 2005.

(51) Int. Cl. A61K 39/395; C12N 5/06; A61K 31/7088; A61K 38/02; A61K 35/12

(52) U.S. Cl. 424/130.1; 435/377; 514/44; 514/2; 514/9; 424/93.21

(57) ABSTRACT

The present invention is based on the discovery that oligodendrocyte-myelin glycoprotein (OMgp), which is ex-pressed by oligodendrocytes and CNS myelin, negatively regulates oligodendrocyte and neuronal differentiation and survival. Based on these discoveries, the invention relates generally to methods of promoting neuronal and oligodendrocyte survival and differentiation by administration of an OMgp anatagonist. Additionally, the invention generally relates to methods of treating various diseases, disorders or injuries associated with demyelination, dysmyelination, oligodendrocyte/neuronal cell death, axonal injury and/or differentiation by the administration of an OMgp antagonist.

### BACKGROUND OF THE INVENTION

1. Field of the Invention

This invention relates to neurobiology, neurology and pharmacology. More particularly, it relates to methods of promoting neuron and oligodendrocyte survival, proliferation and differentiation and methods of treating neuronal diseases related by the administration of OMgp antagonists.

2. Background Art

Damaged neurons do not regenerate in the central nervous system (CNS) following injury due to trauma and disease. The absence of axon regeneration following injury can be attributed to the presence of axon growth inhibitors. These inhibitors are predominantly associated with myelin and constitute an important barrier to regeneration. Axon growth inhibitors are present in CNS-derived myelin and the plasma membrane of oligodendrocytes, which synthesize myelin in the CNS (Schwab et al., (1993) Ann. Rev. Neurosci. 16, 565-595).

CNS myelin is an elaborate extension of the oligodendrocyte cell membrane. A single oligodendrocyte myelinates as many as thirty or more different CNS axonal segments. Oligodendrocyte membrane extensions wrap around the axons in a concentric fashion to form the myelin sheath. Tightly compacted mature myelin consists of parallel layers of bimolecular lipids apposed to layers of hydrated protein. Active myelin synthesis starts in utero and continues for the first two years of human life. Slower synthesis continues through childhood and adolescence while turnover of mature myelin continues at a slower rate throughout adult life. Both developing and mature forms of myelin are susceptible to injury from disease or physical trauma resulting in degradation of the myelin surrounding axons.

Myelin-associated inhibitors appear to be a primary contributor to the failure of CNS axon regeneration in vivo after an interruption of axonal continuity, while other non-myelin associated axon growth inhibitors in the CNS may play a lesser role. These myelin inhibitors block axonal regeneration following neuronal injury due to trauma, stroke, or viral infection. (Brittis et al., 2001, Neuron 30: 11-14; Jones et al, 2002, J. Neurosci. 22: 2792-2803; Grimpe et al, 2002, J. Neurosci. 22: 3144-3160).

Several myelin inhibitory proteins found on oligodendrocytes have been characterized. Known examples of myelin inhibitory proteins include NogoA (Chen et al., Nature, 2000, 403, 434-439; Grandpre et al., Nature 2000, 403, 439-444), myelin associated glycoprotein (MAG) (McKerracher et al., 1994, Neuron 13: 805-811; Mukhopadhyay et al., 1994, Neuron 13: 757-767) and oligodendrocyte-myelin glycoprotein (OMgp), Mikol et al., 1988, J. Cell. Biol. 106: 1273-1279). Each of these proteins has been separately shown to be a ligand for the neuronal NgRl (Wang et al., Nature 2002, 417, 941-944; Grandpre et al., Nature 2000, 403, 439-444; Chen et al., Nature, 2000, 403, 434-439; Domeniconi et al., Neuron 2002, published online Jun. 28, 2002).

Specifically, oligodendrocyte-myelin glycoprotein (OMgp) has a molecular weight of about 120-kD, is glycosylated and is linked to the cellular membrane via a glycosylphosphatidylinositol (GPI) lipid intermediate anchor. OMgp contains a leader sequence of approximately 24 amino acid residues and four structural domains. The N-terminal portion of the protein contains a cysteine-rich region (CR) or an N-terminal leucine rich repeat (LRR) domain about 32 amino acid residues in length, followed by several leucine rich repeat (LRR) domains which span about 190 to 205 amino acid residues in length (including N- and C-terminal caps). The OMgp protein contains approximately six LRR domains. The remaining two domains are a serine-threonine rich (S/TR) region of about 197 amino acids and a hydrophobic COOH-sequence of about 15 amino acids at the C-terminus of OMgp. The S/TR region of OMgp has sites capable of attaching to O-linked carbohydrates. As one of skill in the art would appreciate, the boundaries and number of domains (e.g. the LRR domains) may vary in a polypeptide depending upon which protein structure modeling program is used to analyze the structure of the protein.

OMgp is a member of the CR-LRR protein family due to the inclusion of cysteine-rich and leucine-rich regions within the protein. It is believed that the CR and LRR domains are responsible for the cell adhesion mediated by the proteins in the CR-LRR family. Other examples of proteins in this family are the α and β chains of platelet glycoprotein Ib, biglycan and chaoptin. OMgp has traditionally been considered a component of myelin membranes in the CNS. Recently it has been discovered that OMgp is a component of the thin glial membranes that appear to extend on the outer surface of myelin. (Huang et al. Manuscript in preparation).

There is an unmet need for molecules and methods for promoting neuronal survival, proliferation and differentiation. Particularly for the treatment of disease, disorders or injuries which involve neuronal or oligodendrocyte cell death or generally relate to the nervous system.

Such diseases, disorders or injuries include, but are not limited to, multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Globoid cell Leucodystrophy (Krabbe's disease) and Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, and Bell's palsy. Among these diseases, MS is the most widespread, affecting approximately 2.5 million people worldwide.

### BRIEF SUMMARY OF THE INVENTION

The present invention is based on the discovery that oligodendrocyte-myelin glycoprotein (OMgp) (OMgp is also designated in the literature as Sp37), which is expressed by oligodendrocytes and CNS myelin, negatively regulates oligodendrocyte and neuronal differentiation, proliferation and survival. Neurons from OMgp knock-out mice showed increased survival and earlier onset of differentiation as compared to their wild-type littermates. Based on these discoveries, the invention relates generally to methods of promoting neuronal and oligodendrocyte survival and differentiation by administration of an OMgp antagonist. Additionally, the invention is related generally to methods of treating various diseases, disorders or injuries associated with demyelination, dysmyelination, oligodendrocyte/neuronal cell death, axonal injury and/or differentiation by the administration of an OMgp antagonist.

In certain embodiments, the invention provides for methods of promoting differentiation, proliferation or survival of neurons comprising contacting a neuron with an effective amount of a composition comprising an OMgp antagonist.

The invention also provides for methods of promoting proliferation, differentiation or survival of oligodendrocytes comprising contacting an oligodendrocyte with an effective amount of a composition comprising an OMgp antagonist.

Additionally, the invention provides for methods of promoting myelination of neurons in a mammal comprising administering to a mammal an effective amount of a composition comprising an OMgp antagonist.

In additional embodiments, the invention provides for methods for treating a disease, disorder, or injury associated with neuronal or oligodendrocyte death or lack of proliferation and/or differentiation in a mammal comprising administering to a mammal in need thereof a therapeutically effective amount of a composition comprising an OMgp antagonist.

The invention also relates to methods for treating a disease, disorder, or injury involving the destruction of myelin in a mammal comprising administering a therapeutically effective amount of a composition comprising an OMgp antagonist.

In various embodiments of the above methods, the OMgp antagonist may be any molecule which interferes with ability of OMgp to negatively regulate survival, proliferation and differentiation of neurons and oligodendrocytes, as well as myelination of neurons. In certain embodiments the OMgp antagonist is a soluble OMgp polypeptide, an OMgp antibody or fragment thereof, an OMgp antagonist polynucleotide, or a combination of two or more OMgp antagonists.

In certain embodiments, the OMgp antagonist is a soluble OMgp polypeptide. Certain soluble OMgp polypeptides of the invention comprise or lack the following domains: an OMgp cysteine-rich domain, an OMgp LRR, an OMgp serine/threonine-rich domain, a fragment, variant, or derivative thereof of an OMgp cysteine rich domain, LRR domain or serine/threonine rich domain, or a combination of at least two of said OMgp domains, fragments, variants, or derivatives thereof. In some embodiments, the OMgp antagonist is administered by bolus injection or chronic infusion. In some embodiments, the soluble OMgp polypeptide is administered directly into the central nervous system. In some embodiments, the soluble OMgp polypeptide is administered directly into a chronic lesion of MS.

In other embodiments the OMgp antagonist is a OMgp antibody or fragment thereof which binds to an OMgp polypeptide comprising one or more of the following OMgp regions or domains: an OMgp cysteine-rich domain, an OMgp LRR domain; an OMgp serine/threonine-rich domain. Additionally, the OMgp antibody or fragment thereof specifically binds to an epitope within a polypeptide comprising an OMgp polypeptide fragment as described herein.

In other embodiments, the OMgp antagonist is a OMgp antagonist polynucleotide such as an antisense polynucleotide, a ribozyme, a small interfering RNA (siRNA), or a small-hairpin RNA (shRNA).

In some embodiments, the disease, disorder or injury is multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, or Bell's palsy.

Further embodiments of the invention include methods of treating a disease, disorder or injury by in vivo gene therapy, comprising administering to a mammal, at or near the site of the disease, disorder or injury, a vector comprising a nucleotide sequence that encodes an OMgp antagonist so that the OMgp antagonist is expressed from the nucleotide sequence in the mammal in an amount sufficient to reduce inhibition of neuronal or oligodendrocyte death, proliferation and/or differentiation at or near the site of the injury. In certain embodiments, the vector is a viral vector which is selected from the group consisting of an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, and a herpes simplex viral vector. In some embodiments, the vector is administered by a route selected from the group consisting of topical administration, intraocular administration, parenteral administration, intrathecal administration, subdural administration and subcutaneous administration.

In some embodiments, the OMgp antagonist is a fusion polypeptide comprising a non-OMgp moiety. In some embodiments, the non-OMgp moiety is selected from the group consisting of an antibody Ig moiety, a serum albumin moiety, a targeting moiety, a reporter moiety, and a purification-facilitating moiety. In some embodiments, the antibody Ig moiety is a hinge and Fc moiety.

In some embodiments, the polypeptides and antibodies of the present invention are conjugated to a polymer. In some embodiments, the polymer is selected from the group consisting of a polyalkylene glycol, a sugar polymer, and a polypeptide. In some embodiments, the polyalkylene glycol is polyethylene glycol (PEG). In some embodiments, the polypeptides and antibodies of the present invention are conjugated to 1, 2, 3 or 4 polymers. In some embodiments, the total molecular weight of the polymers is from 5,000 Da to 100,000 Da.

### BRIEF DESCRIPTION OF THE DRAWINGS/FIGURES

FIG. 1 is a schematic drawing of the mature oligodendrocyte-myelin glycoprotein (OMgp). The figure depicts the three major domains in the protein as well as the GPI-anchor which tethers the protein to the lipid membrane.

FIG. 2 is a northern blot which has been probed for OMgp expression in different human tissues. OMgp mRNA is only detected in brain tissue.

FIG. 3 shows the results of RT-PCR experiments to detect OMgp transcription in the brain and spinal cord of rats at different developmental time points. OMgp mRNA transcription is at its highest in adults in both the brain and spinal cord. OMgp expression is not detectable in the brain at embryonic day 14 (E 14) and is very low at embryonic day 18 (E18).

FIG. 4A depicts the strategy to create a OMgp knock-out mouse. The entire OMgp coding sequence was replaced by a green fluorescent protein (GFP)/neomycin resistance (neo) expression cassette.

FIG. 4B is the result of an RT-PCR experiment to verify the genotype of the OMgp knock-out mice. In wild-type brain and spinal cord tissue, OMgp mRNA is present. In the brain and spinal cord tissue of the OMgp knock-out mice, OMgp mRNA was not detected.

FIG. 5 shows the results of a Western blot of protein samples from the brain and spinal cord of wild-type and OMgp knock-out mice. OMgp, Nogo-A and MAG are present in the wild-type mice whereas only Nogo-A and MAG are present in the OMgp knock-out mice.

FIG. 6A and FIG. 6B are graphs depicting the number of apoptotic cells and cells with normal nuclei in hippocampal neuron cultures from wild-type and OMgp knock-out mice.

FIG. 7A and FIG. 73 are graphs depicting the neurite length and number of process-bearing neurons in hippocampal neuron cultures from wild-type and OMgp knock-out mice.

FIG. 8A and FIG. 8B are graphs showing the number of neurons and number of caspase-3-positive cells near the site of spinal cord injury, as described in Example 5, in wild-type and OMgp knock-out mice.

FIG. 9 is a graph showing improved functional recovery of wild-type (+/+) and OMgp knock-out mice (−/−) after complete transection of the spinal cord. Locomotion was evaluated using the Basso Mouse Scale (BMS) for locomotion.

FIGS. 10A and 10B show a decrease in Rho activation after spinal cord injury (SCI) in OMgp knock-out mice. FIG. 10A is a immunoblot showing RhoA GTP in wild-type injured and uninjured mice compared to OMgp knock-out injured and uninjured mice. FIG. 10B is a quantification of RhoA GTP with the level normalized by total Rho. Data are represented as mean±SEM, *p≦0.05.

### DETAILED DESCRIPTION OF THE INVENTION

### Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

In order to further define this invention, the following terms and definitions are provided.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

Throughout this specification and claims, the word “comprise,” or variations such as “comprises” or “comprising,” indicate the inclusion of any recited integer or group of integers but not the exclusion of any other integer or group of integers.

As used herein, a “therapeutically effective amount” refers to an amount effective, at dosages and for periods of time necessary, to achieve a desired therapeutic result. A therapeutic result may be, e.g., lessening of symptoms, prolonged survival, improved mobility, and the like. A therapeutic result need not be a “cure”.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “polynucleotide” can contain the nucleotide sequence of the full length cDNA sequence, including the untranslated 5′ and 3′ sequences, the coding sequences, as well as fragments, epitopes, domains, and variants of the nucleic acid sequence. The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In the present invention, a polypeptide can be composed of amino acids joined to each other by peptide bonds or modified peptide bonds, i.e., peptide isosteres, and may contain amino acids other than the 20 gene-encoded amino acids (e.g. non-naturally occurring amino acids). The polypeptides of the present invention may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

The terms “fragment,” “variant,” “derivative” and “analog” when referring to an OMgp antagonist of the present invention include any antagonist molecules which retain at least some ability to inhibit OMgp activity. OMgp antagonists as described herein may include fragment, variant, or derivative molecules therein without limitation, so long as the OMgp antagonist still serves its function. Soluble OMgp polypeptides of the present invention may include OMgp proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. Soluble OMgp polypeptides of the present invention may comprise variant OMgp regions, including fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Soluble OMgp polypeptides may comprise conservative or non-conservative amino acid substitutions, deletions or additions. OMgp antagonists of the present invention may also include derivative molecules. For example, soluble OMgp polypeptides of the present invention may include OMgp regions which have been altered so as to exhibit additional features not found on the native polypeptide. Examples include fusion proteins and protein conjugates.

In the present invention, a “polypeptide fragment” refers to a short amino acid sequence of an OMgp polypeptide. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids in length.

Antibody or Immunoglobulin. In one embodiment, the OMgp antagonists for use in the treatment methods disclosed herein are “antibody” or “immunoglobulin” molecules, or immunospecific or antigen-binding fragments or antigen-binding fragments thereof, e.g., naturally occurring antibody or immunoglobulin molecules or engineered antibody molecules or fragments that bind antigen in a manner similar to antibody molecules. The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises five broad classes of polypeptides that can be distinguished biochemically. All five classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H1}, C_{H2} or C_{H3}) confer important biological properties such as secretion, transplacental mobility, Fc receptor binding, complement binding, and the like.

By convention the numbering of the constant region domains increases as they become more distal from the antigen binding site or amino-terminus of the antibody. The N-terminal portion is a variable region and at the C-terminal portion is a constant region; the C_{H3} and C_{L} domains actually comprise the carboxy-terminus of the heavy and light chain, respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region allows the antibody to selectively recognize and specifically bind epitopes on antigens. That is, the V_{L} domain and V_{H} domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains. In some instances, e.g., certain immunoglobulin molecules derived from camelid species or engineered based on camelid immunoglobulins, a complete immunoglobulin molecule may consist of heavy chains only, with no light chains. See, e.g., Hamers Casterman et al., Nature 363:446 448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In camelid species, however, the heavy chain variable region, referred to as V_{H}H, forms the entire CDR. The main differences between camelid V_{H}H variable regions and those derived from conventional antibodies (V_{H}) include (a) more hydrophobic amino acids in the light chain contact surface of V_{H} as compared to the corresponding region in V_{H}H, (b) a longer CDR₃ in V_{H}H, and (c) the frequent occurrence of a disulfide bond between CDR₁ and CDR₃ in V_{H}H.

In one embodiment, an antigen binding molecule of the invention comprises at least one heavy or light chain CDR of an antibody molecule. In another embodiment, an antigen binding molecule of the invention comprises at least two CDRs from one or more antibody molecules. In another embodiment, an antigen binding molecule of the invention comprises at least three CDRs from one or more antibody molecules. In another embodiment, an antigen binding molecule of the invention comprises at least four CDRs from one or more antibody molecules. In another embodiment, an antigen binding molecule of the invention comprises at least five CDRs from one or more antibody molecules. In another embodiment, an antigen binding molecule of the invention comprises at least six CDRs from one or more antibody molecules. Exemplary antibody molecules comprising at least one CDR that can be included in the subject antigen binding molecules are known in the art and exemplary molecules are described herein.

Antibodies or immunospecific or antigen-binding fragments thereof for use in the methods of the invention include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to binding molecules disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG₁, IgG₂, IgG₃, IgG₄, IgA₁ and IgA₂) or subclass of immunoglobulin molecule.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. Antibodies or immunospecific or antigen-binding fragments thereof for use in the diagnostic and therapeutic methods disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide for use in the invention may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide of the invention comprises a polypeptide chain comprising a C_{H}3 domain. Further, a binding polypeptide for use in the invention may lack at least a portion of a C_{H}2 domain (e.g., all or part of a C_{H}2 domain). As set forth above, it will be understood by one of ordinary skill in the art that these domains (e.g., the heavy chain portions) may be modified such that they vary in amino acid sequence from the naturally occurring immunoglobulin molecule.

In certain OMgp antagonist antibodies or immunospecific or antigen-binding fragments thereof for use in the treatment methods disclosed herein, the heavy chain portions of one polypeptide chain of a multimer are identical to those on a second polypeptide chain of the multimer. Alternatively, heavy chain portion-containing monomers for use in the methods of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a C_{H}1 domain derived from an IgG₁ molecule and a hinge region derived from an IgG₃ molecule. In another example, a heavy chain portion can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain portion” includes amino acid sequences derived from an immunoglobulin light chain. Preferably, the light chain portion comprises at least one of a V_{L} or C_{L} domain.

An isolated nucleic acid molecule encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

Antibodies or immunospecific or antigen-binding fragments thereof for use in the treatment methods disclosed herein may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibodies or immunospecific or antigen-binding fragments thereof for use in the treatment methods disclosed herein act as antagonists of OMgp as described herein. For example, an antibody for use in the methods of the present invention may function as an antagonist, blocking or inhibiting the suppressive activity of the OMgp polypeptide.

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is biochemical, expression includes the creation of that biochemical and any precursors.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### Oligodendrocyte-Myelin Glycoprotein—OMgp (Sp37)

The invention is based on the discovery that the abrogation of OMgp function increases neuron and oligodendrocyte numbers by promoting their survival, proliferation and differentiation. In addition, antagonists of OMgp may promote myelination of neurons.

The human OMgp gene is located within an intron of the NF1 gene (chromosome location 17q11-12). Viskochil et al., Mol. Cell. Biol. 11:906-912 (1991). Mutations which result in the loss of NF1 function result in the disease neurofibromatosis type I which is characterized by neurofibromas, Lisch nodules, café au lait skin spots and increased risk of malignant neurofibrosarcomas and optic gliomas.

Tissue distribution and developmental expression of OMgp have been studied in human CNS myelin and in ovine oligodendrocytes in culture. See Vourc'h and Andres, Brain Res. Rev. 45:115-124 (2004). Additionally, studies have been performed to examine OMgp mRNA and protein in rodents during development. Northern blot analysis indicated that in mouse brain, OMgp mRNA increases from P0 to P21 and then decreases, reaching a stable level at P24 (Vourc'h and Andres, Brain Res. Rev. 45:115-124 (2004). Additionally, the temporal expression of OMgp mRNA was studied in the development of rat CNS with the observation that OMgp gene expression increases from birth until postnatal week 6 (around P42). See Vourc'h et al., Dev. Brain Res. 144:159-168 (2003). The temporal expression profile of OMgp was also observed in cultures of oligodendrocytes obtained from newborn rat brains. Id. These results suggest that OMgp expression by oligodendrocytes do not depend on axonal signals.

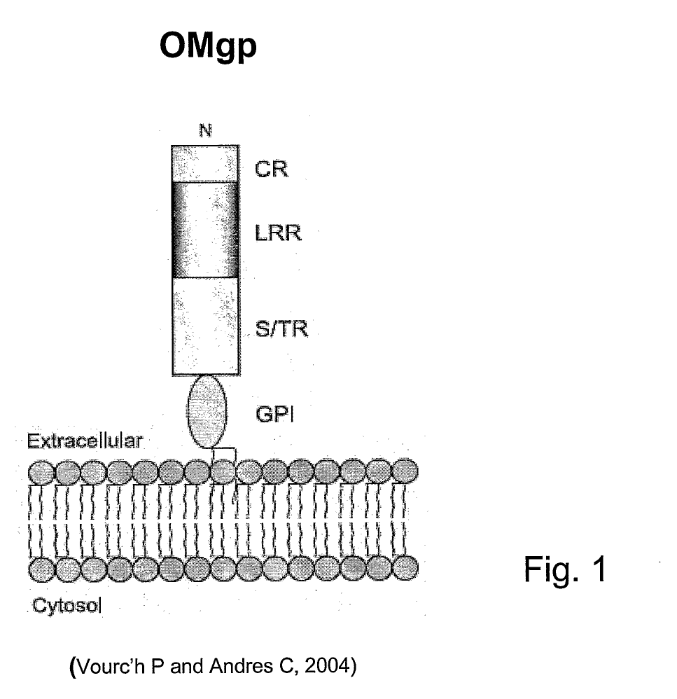

Naturally occurring human OMgp is a 120-kD glycosylated protein which is expressed from a gene that encodes 440 amino acids (SEQ ID NO: 2). OMgp is a member of the Nogo Receptor (NgR) ligand family. The mature form of OMgp is about 401 amino acids. Other members of the NgR ligand family include Myelin-associated glycoprotein (MAG) and Nogo. The human OMgp polypeptide contains a cysteine-rich (CR) domain or N-terminal LRR domain of approximately 32 amino acid residues in length followed by a stretch of 6 or 8 tandem leucine-rich repeats (including the N-terminal cap (LRRNT) and C-terminal cap (LRRCT). The number of predicted repeats may vary depending upon which protein computer modeling program is used. The LRR domains comprise approximately 190 to 205 amino acid residues of the OMgp protein. OMgp also contains a serine-threonine rich (S/TR) domain (which can be separated into several S/T-rich repeats) of about 197 amino acid residues in length. (FIG. 1). The hydrophobic domain is cleaved prior to the attachment of a glycosyphostadidylinositol (GPI) link to the C-terminus of the protein. OMgp does not contain a transmembrane domain and is attached to the outer layer of the plasma membrane via the GPI anchor. The LRR domains contain the majority of the protein's glycosylation sites, while the S/TR domain contains several putative sites for O— and N— carbohydrate modification. In addition, the naturally occurring OMgp protein contains a signal sequence at the N-terminus of the protein which is about 24 amino acids in length. Table 1 lists the OMgp domains according to amino acid residue number, based on the sequence of SEQ ID NO:2 or SEQ ID NO:4.

TABLE 1

| Domain or Region | Beginning Residue | Ending Residue |
|---|---|---|
| Signal Sequence | 1 | 24 |
| CR or LRRNT | 25 | 54 or 56 |
| LRR1 | 55 or 57 | 75 |
| LRR2 | 79 | 98 |
| LRR3 | 147 | 166 |
| LRR4 | 168 | 191 |
| LRRCT | 192 | 215 OR 228 |
| S/TR | 216 or 229 | 425 |
| COOH-sequence | 426 | 440 |

Several polynucleotide and polypeptide sequences have been reported for human OMgp. These sequences are reproduced below. The following polynucleotide and polypeptide sequence was reported as the mRNA for human OMgp and is accession number BC018050 in Genbank:

(SEQ ID NO:1)

gttgaagacg acaccacggc tttgatggaa tatcagatat
tgaaaatgtc tctctgcctg ttcatccttc tgtttctcac
acctggtatt ttatgcattt gtcctctcca atgtatatgc
acagagaggc acaggcatgt ggactgttca ggcagaaact
tgtctacatt accatctgga ctgcaagaga atattataca
tttaaacctg tcttataacc actttactga tctgcataac
cagttaaccc aatataccaa tctgaggacc ctggacattt
caaacaacag gcttgaaagc ctgcctgctc acttacctcg
gtctctgtgg aacatgtctg ctgctaacaa caacattaaa
cttcttgaca aatctgatac tgcttatcag tggaatctta
aatatctgga tgtttctaag aacatgctgg aaaaggttgt
cctcattaaa aatacactaa gaagtctcga ggttctcaac
ctcagtagta acaaactttg gacagttcca accaacatgc
cctccaaact acatatcgtg gacctgtcta ataattcttt
gacacaaatt cttccaggta cattaataaa cctgacaaat
ctcacacatc tttacctgca caacaataag ttcacattca
ttccagacca atcttttgac caactctttc agttgcaaga
gataaccctt tacaataaca ggtggtcatg tgaccacaaa
caaaacatta cttacttact gaagtggatg atggaaacaa
aagcccatgt gatagggact ccatgttcta cccaaatatc
atctttaaag gaacataaca tgtatcccac accttctgga
tttacctcaa gcttattcac tgtaagtggg atgcagacag
tggacaccat taactctctg agtgtggtaa ctcaacccaa
agtgaccaaa atacccaaac aatatcgaac aaaggaaaca
acgtttggtg ccactctaag caaagacacc acctttacta
gcactgataa ggcttttgtg ccctatccag aagatacatc
cacagagact atcaattcac atgaagcagc agctgcaact
ctaactatct atctccaaga tggaatggtc acaaacacaa
gcctcactag ctcaaacaaa tcatccccaa cacccatgac
cctaagtatc actagtggca tgccaaataa tttctctgaa
atgcctcaac aaagcacaac ccttaactta tggagggaag
agacaaccac aaatgtaaag actccattac cttctgtggc
aaatgcttgg aaagtaaatg cttcatttct cttattgctc
aatgttgtgg tcatgctggc tgtctgaggg tctgcatttt
ctgaaactaa tgaaagcact cctccctgat gtacagttgg
gaaaatatgt ccatatctaa ccagtgattc gagctatatt
taagtattca agaaagccag tcttaacatt tctaactctg
atgtaaatga agtaaottgt cttaaataaa agaaatgcac
aatgtcttgg tacttgctgc tattttactg tcttaattaa
gtaaactaat gagtttctct tataaaaaaa atgaaatgtt
ttaaggcttc aatttattgc acaaaatata aagcatctaa
actttaatat gtattttatg tatgtttaca ctgtcaaaca
tctggaaaat aaaaggtcta tgctcaaaaa aaaaaaaaaa
aaaaaaaaaa aaaaaaaaaa aaaaaaaaaa aaaaaa

(SEQ ID NO:2)

MEYQILKMSLCLFILLFLTPGILCICPLQCICTERHRHVDCSGRNLSTLP
SGLQENIIHLNLSYNHFTDLHNQLTQYTNLRTLDISNNRLESLPAHLPRS
LWNMSAANNNIKLLDKSDTAYQWNLKYLDVSKNMLEKVVLIKNTLRSLEV
LNLSSNKLWTVPTNMPSKLHIVDLSNNSLTQILPGTLINLTNLTHLYLHN
NKFTFIPDQSFDQLFQLQEITLYNNRWSCDHKQNITYLLKWMMETKAHVI
GTPCSTQISSLKEHNMYPTPSGFTSSLFTVSGMQTVDTINSLSVVTQPKV
TKIPKQYRTKETTFGATLSKDTTFTSTDKAFVPYPEDTSTETINSHEAAA
ATLTIHLQDGMVTNTSLTSSTKSSPTPMTLSITSGMPNNFSEMPQQSTTL
NLWREETTTNVKTPLPSVANAWKVNASFLLLLNVVVMLAV

The following polynucleotide and polypeptide sequence was reported as the mRNA for human OMgp and is accession number NM_{—}002544 in Genbank:

(SEQ ID NO:3)

tgaggcaaat gttaatgagg caatgttaaa tatggaccca
atgtcagaca aatacataga aaggagtaag ggccaactct
catgcataag gtatcccatc ctatagcaaa tcagatatat
aggtacgctt gatgccacaa attttttaaa aaattgtcca
ttttgttgcg tgtgcacctc ttgccataaa tttgagtcag
caccagcgac agctctgcag tcctcctatg tggtactgat
caggtggttg cagagcttca gctcacagca acacaatgca
gctgagcagg caagcacagc ccacagccag aaacagttcc
gactctacag aacaagacga cctttaagtt tcccagagaa
aatgagatgc tgatgttgaa gacgacacca cgggtaagat
gttatttaaa tcagtaaaag gctgactttg gaatcttttt
cctttttctt ttaagaaaaa gtcaacgtta ggattaaata
tatattcaat agcaagtgca tgcaccagaa atttgctgca
gtgtcagttg agggatattt tttatacatt cagtcactct
gtaaatatac atattgtttt cctttaaaat gggcactgaa
atatacagaa aaaaatcact ttataaaatg tgaggtttat
aggtactgtg ttggtctgga tttttcaagt gctttttaca
aagatatatt tatcctaaaa acatacagat aaaaatttcg
aagactgctt taatatctaa ataaaatcta ccctatatac
acacattgaa ttacattacc tgcagagatt aaaaaaaaaa
gacacgacag ccatttttct catctgagta agaaagcata
tcatcaaaaa tagtaatagc ctacaactgc aactatttat
ttgcaaagaa tgctatttta tcatattaag gctctagaaa
gataaataag aaagaatatg gttagaaaag gggggaggga
gagagaaaat aaaggagaaa atgcaggaga gagtagggag
agagtctctc tctaccacat agcccaatga aggattaagc
attgactata aatgaaggga gctttgttag tttaatcact
ggaacaatta taaaaggact cgacaacaac gaggtttatt
gaaaattttg cctaatgcta actgacccat gcagatgcct
aaactgtatt tgcatattaa aagaagggtg tatctgtttg
tttctaggct ttgatggaat atcagatatt gaaaatgtct
ctctgcctgt tcatccttct gtttctcaca cctgntattt
tatgcatttg tcctctccaa tgtatatgca cagagaggca
caggcatgtg gactgttcag gcagaaactt gtctacatta
ccatctggac tgcaagagaa tattatacat ttaaacctgt
cttataacca ctttactgat ctgcataacc agttaaccca
atataccaat ctgaggaccc tggacatttc aaacaacagg
cttgaaagcc tgcctgctca cttacctcgg tctctgtgga
acatgtctgc tgctaacaac aacattaaac ttcttgacaa
atctgatact gcttatcagt ggaatcttaa atatctggat
gtttctaaga acatgctgga aaaggttgtc ctcattaaaa
atacactaag aagtctcgag gttctcaacc tcagtagtaa
caaactttgg acagttccaa ccaacatgcc ctccaaacta
catatcgtgg acctgtctaa taattctttg acacaaattc
ttccaggtac attaataaac ctgacaaatc tcacacatct
ttacctgcac aacaataagt tcacattcat tccagaccaa
tcttttgacc aactctttca gttgcaagag ataacccttt
acaataacag gtggtcatgt gaccacaaac aaaacattac
ttacttactg aagtggatga tggaaacaaa agcccatgtg
atagggactc catgttctac ccaaatatca tctttaaagg
aacataacat gtatcccaca ccttctggat ttacctcaag
cttattcact gtaagtggga tgcagacagt ggacaccatt
aactctctga gtgtggtaac tcaacccaaa gtgaccaaaa
tacccaaaca atatcgaaca aaggaaacaa cgtttggtgc
cactctaagc aaagacacca cctttactag cactgataag
gcttttgtgc cctatccaga agatacatcc acagagacta
tcaattcaca tgaagcagca gctgcaactc taactattca
tctccaagat ggaatggtca caaacacaag cctcactagc
tcaacaaaat catccccaac acccatgacc ctaagtatca
ctagtggcat gccaaataat ttctctgaaa tgcctcaaca
aagcacaacc cttaacttat ggagggaaga gacaaccaca
aatgtaaaga ctccattacc ttctgtggca aatgcttgga
aagtaaatgc ttcatttctc ttattgctca atgttgtggt
catgctggct gtctgagggt ctgcattttc tgaaactaat
gaaagcactc ctccctgatg tacagttggg aaaatatgtc
catatctaac cagtgattcg agctatattt aagtattcaa
gaaagccagt cttaacattt ctaactctga tgtaaatgaa
gtaacttgtc ttaaataaaa gaaatgcaca atgtcttggt
acttgctgct attttactgt cttaattaag taaactaatg
agtttctttt ataaaaaaaa tgaaatgttt taaggcttca
atttattgca caaaatataa agcatctaaa ctttaatatg
tattttatgt atgtttacac tgtcaaacat ctggaaaata
aaaggtctat gctcataact gtgtcatttg gctttccagt
cataccaact ttnagcagaa tcaaaatgac ctcaccattt
ttgttctaggg at

(SEQ ID NO:4)

MEYQILKMSLCLFILLFLTPXILCICPLQCICTERHRHVDCSGRNLSTLP
SGLQENIIHLNLSYNHFTDLHNQLTQYTNLRTLDISNNRLESLPAHLPRS
LWNMSAANNNIKLLDKSDTAYQWNLKYLDVSKNMLEKVVLIKNTLRSLEV
LNLSSNKLWTVPTNMPSKLHIVDLSNNSLTQILPGTLINLTNLTHLYLHN
NKFTFIPDQSFDQLFQLQEITLYNNRWSCDHKQNITYLLKWMMETKAHVI
GTPCSTQISSLKEHNMYPTPSGFTSSLFTVSGMQTVDTINSLSVVTQPKV
TKIPKQYRTKETTFGATLSKDTTFTSTDKAFVPYPEDTSTETINSHEAAA
ATLTIHLQDGMVTNTSLTSSTKSSPTPMTLSITSGMPNNFSEMPQQSTTL
NLWREETTTNVKTPLPSVANAWKVNASFLLLLNVVVMLAV

The following polynucleotide and polypeptide sequence was reported as the in RNA for human OMgp and is accession number X51694 in Genbank:

(SEQ ID NO:5)

gttccgactc tacagaacaa gacgaccttt aagtttccca
gagaaaatga gatgctgatg ttgaagacga caccacggct
ttgatggaat atcagatatt gaaaatgtct ctctgcctgt
tcatccttct gtttctcaca cctgntattt tatgcatttg
tcctctccaa tgtatatgca cagagaggca caggcatgtg
gactgttcag gcagaaactt gtctacatta ccatctggac
tgcaagagaa tattatacat ttaaacctgt cttataacca
ctttactgat ctgcataacc agttaaccca atataccaat
ctgaggaccc tggacatttc aaacaacagg cttgaaagcc
tgcctgctca cttacctcgg tctctgtgga acatgtctgc
tgctaacaac aacattaaac ttcttgacaa atctgatact
gcttatcagt ggaatcttaa atatctggat gtttctaaga
acatgctgga aaaggttgtc ctcattaaaa atacactaag
aagtctcgag gttctcaacc tcagtagtaa caaactttgg
acagttccaa ccaacatgcc ctccaaacta catatcgtgg
acctgtctaa taattctttg acacaaattc ttccaggtac
attaataaac ctgacaaatc tcacacatct ttacctgcac
aacaataagt tcacattcat tccagaccaa tcttttgacc
aactctttca gttgcaagag ataacccttt acaataacag
gtggtcatgt gaccacaaac aaaacattac ttacttactg
aagtggatga tggaaacaaa agcccatgtg atagggactc
catgttctac ccaaatatca tctttaaagg aacataacat
gtatcccaca ccttctggat ttacctcaag cttattcact
gtaagtggga tgcagacagt ggacaccatt aactctctga
gtgtggtaac tcaacccaaa gtgaccaaaa tacccaaaca
atatcgaaca aaggaaacaa cgtttggtgc cactctaagc
aaagacacca cctttactag cactgataag gcttttgtgc
cctatccaga agatacatcc acagagacta tcaattcaca
tgaagcagca gctgcaactc taactattca tctccaagat
ggaatggtca caaacacaag cctcactagc tcaacaaaat
catccccaac acccatgacc ctaagtatca ctagtggcat
gccaaataat ttctctgaaa tgcctcaaca aagcacaacc
cttaacttat ggagggaaga gacaaccaca aatgtaaaga
ctccattacc ttctgtggca aatgcttgga aagtaaatgc
ttcatttctc ttattgctca atgttgtggt catgctggct
gtctgagggt ctgcattttc tgaaactaat gaaagcactc
ctccctgatg tacagttggg aaaatatgtc catatctaac
cagtgattcg agctatattt aagtattcaa gaaagccagt
cttaacattt ctaactctga tgtaaatgaa gtaacttgtc
ttaaataaaa gaaatgcaca atgtcttggt acttgctgct
attttactgt cttaattaag taaactaatg agtttctttt
ataaaaaaaa tgaaatgttt taaggcttca atttattgca
caaaatataa agcatctaaa ctttaatatg tattttatgt
atgtttacac tgtcaaacat ctggaaaata aaaggtctat
gctcaaaaaa aaaaaaaaaa aaaaaaaaaa aaaaaaaaaa
aaaaaaaa

(SEQ ID NO:6)

MSLCLFILLFLTPXILCICPLQCICTERHRHVDCSGRNLSTLPSGLQENI
IHLNLSYNHFTDLHNQLTQYTNLRTLDISNNRLESLPAHLPRSLWNMSAA
NNNIKLLDKSDTAYQWNLKYLDVSKNMLEKVVLIKNTLRSLEVLNLSSNK
LWTVPTNMPSKLHIVDLSNNSLTQILPGTLINLTNLTHLYLHNNKFTFIP
DQSFDQLFQLQEITLYNNRWSCDHKQNITYLLKWMMETKAHVIGTPCSTQ
ISSLKEHNMYPTPSGFTSSLFTVSGMQTVDTINSLSVVTQPKVTKIPKQY
RTKETTFGATLSKDTTFTSTDKAFVPYPEDTSTETINSHEAAAATLTIHL
QDGMVTNTSLTSSTKSSPTPMTLSITSGMPNNFSEMPQQSTTLNLWREET
TTNVKTPLPSVANAWKVNASFLLLLNVVVMLAV

The following polynucleotide and polypeptide sequence was reported as the mRNA for human OMgp and is accession number XM_{—}083979 in Genbank:

(SEQ ID NO:7)

aatgcagctg agcaggcaag cacagcccac agccagaaac
agttccgact ctacagaaca agacgacctt taagtttccc
agagaaaatg agatgctgat gttgaagacg acaccacggc
tttgatggaa tatcagatat tgaaaatgtc tctctgcctg
ttcatccttc tgtttctcac acctggtatt ttatgcattt
gtcctctcca atgtatatgc acagagaggc acaggcatgt
ggactgttca ggcagaaact tgtctacatt accatctgga
ctgcaagaga atattataca tttaaacctg tcttataacc
actttactga tctgcataac cagttaaccc aatataccaa
tctgaggacc ctggacattt caaacaacag gcttgaaagc
ctgcctgctc acttacctcg gtctctgtgg aacatgtctg
ctgctaacaa caacattaaa cttcttgaca aatctgatac
tgcttatcag tggaatctta aatatctgga tgtttctaag
aacatgctgg aaaaggttgt cctcattaaa aatacactaa
gaagtctcga ggttctcaac ctcagtagta acaaactttg
gacagttcca accaacatgc cctccaaact acatatcgtg
gacctgtcta ataattcttt gacacaaatt cttccaggta
cattaataaa cctgacaaat ctcacacatc tttacctgca
caacaataag ttcacattca ttccagacca atcttttgac
caactctttc agttgcaaga gataaccctt tacaataaca
ggtggtcatg tgaccacaaa caaaacatta cttacttact
gaagtggatg atggaaacaa aagcccatgt gatagggact
ccatgttcta cccaaatatc atctttaaag gaacataaca
tgtatcccac accttctgga tttacctcaa gattattcac
tgtaagtggg atgcagacag tggacaccat taactctctg
agtgtggtaa ctcaacccaa agtgaccaaa atacccaaac
aatatcgaac aaaggaaaca acgtttggtg ccactctaag
caaagacacc acctttacta gcactgataa ggcttttgtg
ccctatccag aagatacatc cacagagact atcaattcac
atgaagcagc agctgcaact ctaactattc atctccaaga
tggaatggtc acaaacacaa gcctcactag ctcaacaaaa
tcatccccaa cacccatgac cctaagtatc actagtggca
tgccaaataa tttctctgaa atgcctcaac aaagcacaac
ccttaactta tggagggaag agacaaccac aaatgtaaag
actccattac cttctgtggc aaatgcttgg aaagtaaatg
cttcatttct cttattgctc aatgttgtgg tcatgctggc
tgtctgaggg tctgcatttt ctgaaactaa tgaaagcact
cctccctgat gtacagttgg gaaaatatgt ccatatctaa
ccagtgattc gagctatatt taagtattca agaaagccag
tcttaacatt tctaactctg atgtaaatga agtaacttgt
cttaaataaa agaaatgcac aatgtcttgg tacttgctgc
tattttactg tcttaattaa gtaaactaat gagtttcttt
tataaaaaaa atgaaatgtt ttaaggcttc aatttattgc
acaaaatata aagcatctaa actttaatat gtattttatg
tatgtttaca ctgtcaaaca tctggaaaat aaaaggtcta
tgctc

(SEQ ID NO:8)

MSLCLFILLFLTPGILCICPLQCICTERHRHVDCSGRNLSTLPSGLQENI
IHLNLSYNHFTDLHNQLTQYTNLRTLDISNNRLESLPAHLPRSLWNMSAA
NNNIKLLDKSDTAYQWNLKYLDVSKNMLEKVVLIKNTLRSLEVLNLSSNK
LWTVPTNMPSKLHIVDLSNNSLTQILPGTLINLTNLTHLYLHNNKFTFIP
DQSFDQLFQLQEITLYNNTWSCDHKQNITYLLKWMMETKAHVIGTPCSTQ
ISSLKEHNMYPTPSGFTSSLFTVSGMQTVDTINSLSVVTQPKVTKIPKQY
RTKETTFGATLSKDTTFTSTDKAFVPYPEDTSTETINSHEAAAATLTIHL
QDGMVTNTSLTSSTKSSPTPMTLSITSGMPNNFSEMPQQSTTLNLWREET
TTNVKTPLPSVANAWKVNASFLLLLNVVVMLAV

The following polynucleotide and polypeptide sequence was reported as the mRNA for mouse OMgp and is accession number NM_{—}019409 in Genbank:

(SEQ ID NO:9)

ctgagctggc aagcagagcc cacagccaga aacccttccg
actcccacaa caagacgacc tttaagctgc aagtttcccg
gagaaaatga gatactgata gtgaagacga cattatgggc
tttgatggaa tatcagatac tgaaaatgtc ttcctgcctg
ttcatccttc tgtttctcac gcctggcatc ttatgcattt
gtcctctcca gtgtacatgc acagagaggc acaggcatgt
ggactgttca ggcagaaact tgactacatt accacctgga
ctgcaggaga acattataca tttaaacctg tcttataacc
actttactga tctgcataac cagttaaccc catataccaa
tctgagaacc ctggatattt caaacaacag gcttgaaagt
ctgcctgctc agttacctcg gtctctctgg aacatgtctg
ctgctaacaa caatattaaa cttcttgaca aatctgatac
tgcttatcag tggaacctta aatacctgga tgtttctaag
aatatgctgg aaaaggttgt tctcattaaa aataccctaa
gaagtctcga ggttcttaac ctcagcagta acaagctttg
gacagttcca accaacatgc cttccaaact gcatatcgtg
gacctgtcta ataactcact gacacaaatc cttccaggga
cattaataaa cctgacaaat ctcacacatc tttacctgca
caacaataaa ttcacattca ttccagaaca gtcttttgac
caacttttgc agttgcaaga gataactctt cataataaca
ggtggtcatg tgaccataaa caaaacatta cttacttatt
gaagtgggtg atggaaacga aagcccatgt gatagggact
ccttgttcta agcaagtatc ctctctaaag gaacagagca
tgtaccccac acctcctggg tttacctcaa gcttatttac
tatgagtgag atgcagacag tggacaccat taactctttg
agtatggtaa ctcaacccaa agtgaccaaa acacccaaac
aatatcgagg aaaggaaacc acatttggtg tcactctaag
caaagatacc acttttagta gcactgatag ggctgtggtg
gcctacccag aagacacacc cacagaaatg accaattccc
atgaagcagc agctgcaact ctaactattc acatacagga
tggaatgagt tcaaatgcaa gcctcaccag tgcaacaaag
tcacccccaa gccccgtgac cctcagcata gctcgtggca
tgccaaataa cttctctgaa atgcctcgac aaagcacaac
cctcaactta cggagggaag aaaccactgc aaatggaaac
actcggccac cttctgcggc tagtgcttgg aaagtaaatg
cctcgctcct tttaatgctc aatgctgtgg tcatgctggc
aggctgaggg tctgcagttt ctgaaacgaa ggagaacctt
cctccatgat gtacagttgg gaaaacgtgc ccctatctaa
ccagtgattc aagctatatt atgtattcaa gaaagccagt
cttatatttc tgactttgat gtaaatgaag taatttgtct
taattaaaag aagtgcacaa tgtcttggta cttgctgcta
ttttcctgtc ttaagtaaaa ctaatgactt ttttttttaa
tgaaatgttt tctttttaag gcttcaactt attgcacaaa
ctataaagag catctaaact ttaatatgta ttttatgtat
gtttacactg tcaaatgtct gggacaaaat aaaa

(SEQ ID NO:10)

MEYQILKMSSCLFILLFLTPGILCICPLQCTCTERHRHVDCSGRNLTTLP
PGLQENIIHLNLSYNHFTDLHNQLTPYTNLRTLDISNNRLESLPAQLPRS
LWNMSAANNNIKLLDKSDTAYQWNLKYLDVSKNMLEKVVLIKNTLRSLEV
LNLSSNKLWTVPTNMPSKLHIVDLSNNSLTQILPGTLINLTNLTNLYLHN
NKFTFIPEQSFDQLLQLQEITLHNNRWSCDHKQNITYLLKWVMETKAHVI
GTPCSKQVSSLKEQSMYPTPPGFTSSLFTMSEMQTVDTINSLSMVTQPKV
TKTPKQYRGKETTFGVTLSKDTTFSSTDRAVVAYPEDTPTEMTNSHEAAA
ATLTIHLQDGMSSNASLTSATKSPPSPVTLSIARGMPNNFSEMPRQSTTL
NLRREETTANGNTRPPSAASAWKVNASLLLMLNAVVMLAG

### Treatment Methods Using Antagonists of OMgp

One embodiment of the present invention provides methods for promoting differentiation, proliferation or survival of a neuron or oligodendrocyte comprising, consisting essentially of, or consisting of contacting said neuron or oligodendrocyte with an effective amount of a composition comprising an OMgp antagonist selected from the group consisting of a soluble OMgp polypeptide, an OMgp antibody and an OMgp antagonist polynucleotide, or any combination thereof.

One embodiment of the present invention provides methods for treating a disease, disorder or injury associated with neuronal or oligodendrocyte death or lack of proliferation or differentiation, e.g., multiple sclerosis, Pelizaeus Merzbacher disease or globoid cell leukodystrophy (Krabbe's disease), in a mammal suffering from such disease. The method comprising, consisting essentially of, or consisting of administering to the mammal a therapeutically effective amount of a composition comprising an OMgp antagonist selected from the group consisting of a soluble OMgp polypeptide, an OMgp antibody and an OMgp antagonist polynucleotide, or any combination thereof.

Further embodiments of the invention include a method of inducing neuronal or oligodendrocyte proliferation, differentiation or survival to treat a disease, disorder or injury involving the destruction of neurons, oligodendrocytes or myelin comprising administering to a mammal, at or near the site of the disease, disorder or injury, in an amount sufficient to reduce inhibition of differentiation, proliferation and survival and promote myelination.

An additional embodiment of the present invention provides methods for treating a disease, disorder or injury associated with dysmyelination or demyelination, e.g., multiple sclerosis, in a mammal suffering from such disease, the method comprising, consisting essentially of, or consisting of administering to the mammal a therapeutically effective amount of a composition comprising an OMgp antagonist selected from the group consisting of a soluble OMgp polypeptide, an OMgp antibody and an OMgp antagonist polynucleotide, or any combination thereof.

Additionally, the invention is directed to a method for promoting myelination of neurons in a mammal comprising, consisting essentially of, or consisting of administering a therapeutically effective amount of a composition comprising an OMgp antagonist selected from the group consisting of a soluble OMgp polypeptide, an OMgp antibody and an OMgp antagonist polynucleotide, or any combination thereof.

An OMgp antagonist, e.g., a soluble OMgp polypeptide, an OMgp antibody or an OMgp antagonist polynucleotide, to be used in treatment methods disclosed herein can be prepared and used as a therapeutic agent that stops, reduces, prevents, or inhibits the ability of OMgp to negatively regulate neuron and oligodendrocyte proliferation, differentiation and survival. Additionally, the OMgp antagonist to be used in treatment methods disclosed herein, can be prepared and used as a therapeutic agent that stops, reduces, prevents, or inhibits the ability of OMgp to negatively regulate myelination of neurons by oligodendrocytes.

In methods of the present invention, an OMgp antagonist can be administered via direct administration of a soluble OMgp polypeptide, OMgp antibody or OMgp antagonist polynucleotide to the patient. Alternatively, the OMgp antagonist can be administered via an expression vector which produces the specific OMgp antagonist. In certain embodiments of the invention, an OMgp antagonist is administered in a treatment method that includes: (1) transforming or transfecting an implantable host cell with a nucleic acid, e.g., a vector, that expresses an OMgp antagonist; and (2) implanting the transformed host cell into a mammal, at the site of a disease, disorder or injury. For example, the transformed host cell can be implanted at the site of a chronic lesion of MS. In some embodiments of the invention, the implantable host cell is removed from a mammal, temporarily cultured, transformed or transfected with an isolated nucleic acid encoding an OMgp antagonist, and implanted back into the same mammal from which it was removed. The cell can be, but is not required to be, removed from the same site at which it is implanted. Such embodiments, sometimes known as ex vivo gene therapy, can provide a continuous supply of the OMgp antagonist, localized at the site of action, for a limited period of time.

Diseases or disorders which may be treated or ameliorated by the methods of the present invention include diseases, disorders or injuries which relate to the death or lack of differentiation or proliferation of neurons or oligodendrocytes. Such disease include, but are not limited to, multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), globoid cell leukodystrophy (Krabbe's disease) and Wallerian Degeneration.

Diseases or disorders which may be treated or ameliorated by the methods of the present invention include diseases, disorders or injuries which relate to dysmyelination or demyelination of mammalian neurons. Specifically, diseases and disorders in which the myelin which surrounds the neuron is either absent, incomplete, not formed properly or is deteriorating. Such disease include, but are not limited to, multiple sclerosis (MS) including relapsing remitting, secondary progressive and primary progressive forms of MS; progressive multifocal leukoencephalopathy (PML), encephalomyelitis (EPL), central pontine myelolysis (CPM), adrenoleukodystrophy, Alexander's disease, Pelizaeus Merzbacher disease (PMZ), globoid cell leukodystrophy (Krabbe's disease), Wallerian Degeneration, optic neuritis and transvere myelitis.

Diseases or disorders which may be treated or ameliorated by the methods of the present invention include neurodegenerate disease or disorders. Such diseases include, but are not limited to, amyotrophic lateral sclerosis, Huntington's disease, Alzheimer's disease and Parkinson's disease.

Examples of additional diseases, disorders or injuries which may be treated or ameliorated by the methods of the present invention include, but are not limited, to spinal cord injuries, chronic myelopathy or rediculopathy, traumatic brain injury, motor neuron disease, axonal shearing, contusions, paralysis, post radiation damage or other neurological complications of chemotherapy, stroke, large lacunes, medium to large vessel occlusions, leukoariaosis, acute ischemic optic neuropathy, vitamin E deficiency (isolated deficiency syndrome, AR, Bassen-Kornzweig syndrome), B12, B6 (pyridoxine-pellagra), thiamine, folate, nicotinic acid deficiency, Marchiafava-Bignami syndrome, Metachromatic Leukodystrophy, Trigeminal neuralgia, Bell's palsy, or any neural injury which would require axonal regeneration, remylination or oligodendrocyte survival, proliferation or differentiation.

### Soluble OMgp Polypeptides

OMgp antagonists of the present invention include those polypeptides which block, inhibit or interfere with the biological function of naturally occurring OMgp. Specifically, soluble OMgp polypeptides of the present invention include fragments, variants, or derivatives thereof of a soluble OMgp polypeptide. Table 1 above describes the various domains of the OMgp polypeptide. Soluble OMgp polypeptides include, but are not limited to, OMgp polypeptides which contain single or multiple domains of the protein as described in Table 1 and FIG. 1. Soluble OMgp polypeptides of the invention also include OMgp domains in various combinations.

A non-limiting example of soluble OMgp polypeptides include polypeptides which comprise, consist essentially of, or consist of one or more of an LRR domain, CR domain or S/TR domain of the OMgp polypeptide. As one of skill in the art would appreciate, any described domain of OMgp may comprise additional or fewer amino acids on either the C-terminal or N-terminal end of the extracellular domain polypeptide. As such, soluble OMgp polypeptides for use in the methods of the present invention include, but are not limited to, an OMgp polypeptide comprising, consisting essentially of, or consisting of amino acids 1 to 54 of SEQ ID NO:2; amino acids 1 to 56 of SEQ ID NO:2; amino acids 1 to 75 of SEQ ID NO:2; amino acids 1 to 98 of SEQ ID NO:2; amino acids 1 to 166 of SEQ ID NO:2; amino acids 1 to 191 of SEQ ID NO:2; amino acids 1 to 215 of SEQ ID NO:2; amino acids 1 to 228 of SEQ ID NO:2; amino acids 1 to 425 of SEQ ID NO:2; amino acids 1 to 440 of SEQ ID NO:2; amino acids 25 to 54 of SEQ ID NO:2; amino acids 25 to 56 of SEQ ID NO:2; amino acids 25 to 75 of SEQ ID NO:2; amino acids 25 to 98 of SEQ ID NO:2; amino acids 25 to 166 of SEQ ID NO:2; amino acids 25 to 191 of SEQ ID NO:2; amino acids 25 to 215 of SEQ ID NO:2; amino acids 25 to 228 of SEQ ID NO:2; amino acids 25 to 425 of SEQ ID NO:2; and amino acids 25 to 440 of SEQ ID NO:2 or fragments, variants, or derivatives of such polypeptides.

A non-limiting example of soluble OMgp polypeptides include polypeptides which comprise, consist essentially of, or consist of various LRR domains. For example OMgp polypeptide which comprise, consist essentially of, or consist of amino acids 55 to 75 of SEQ ID NO:2; amino acids 57 to 75 of SEQ ID NO:2; amino acids 79 to 98 of SEQ ID NO:2; amino acids 147 to 166 of SEQ ID NO:2; amino acids 168 to 191 of SEQ ID NO:2; amino acids 192 to 215 of SEQ ID NO:2; and amino acids 192 to 228 of SEQ ID NO:2.

Additional soluble OMgp polypeptides for use in the methods of the present invention include, but are not limited to, an OMgp polypeptide comprising multiple domains as described in Table 1 and FIG. 1 in various combinations. As such, soluble OMgp polypeptides for use in the present invention include, but are not limited to polypeptides comprising, consisting essentially of, or consisting of amino acids 55 to 98 of SEQ ID NO:2; amino acids 55 to 166 of SEQ ID NO:2; amino acids 55 to 191 of SEQ ID NO:2; amino acids 55 to 215 of SEQ ID NO:2; amino acids 55 to 228 of SEQ ID NO:2; amino acids 57 to 98 of SEQ ID NO:2; amino acids 57 to 166 of SEQ ID NO:2; amino acids 57 to 191 of SEQ ID NO:2; amino acids 57 to 215 of SEQ ID NO:2; amino acids 57 to 228 of SEQ ID NO:2; amino acids 216 to 425 of SEQ ID NO:2; amino acids 229 to 425 of SEQ ID NO:2; amino acids 426 to 440 of SEQ ID NO:2; amino acids 55 to 425 of SEQ ID NO:2; amino acids 57 to 425 of SEQ ID NO:2; amino acids 216 to 440 of SEQ ID NO:2; and amino acids 229 to 440 of SEQ ID NO:2; or fragments, variants, or derivatives of such polypeptides.

Full length OMgp is toxic to cells when expressed constitutively. As such, soluble fragments for use in the present invention are either non-cytoxic when expressed in a cell or can be expressed under the control of an inducible promoter (e.g. a tetracycline regulatable promoter). Such inducible expression systems are known in the art and are also described herein.

Soluble OMgp polypeptides for use in the methods of the present invention described herein may be cyclic. Cyclization of the soluble OMgp polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two ω-thio amino acid residues (e.g. cysteine, homocysteine). Certain soluble OMgp peptides of the present invention include modifications on the N- and C-terminus of the peptide to form a cyclic OMgp polypeptide. Such modifications include, but are not limited, to cysteine residues, acetylated cysteine residues, cysteine residues with a NH₂ moiety and biotin. Other methods of peptide cyclization are described in Li & Roller. Curr. Top. Med. Chem. 3:325-341 (2002), which is incorporated by reference herein in its entirety.

Cyclic OMgp polypeptides for use in the methods of the present invention described herein include, but are not limited to, C₁XX_{N}C₂ where X is any amino acid, N is an number of amino acids, C₁ is a cysteine residue and optionally has a moiety to promote cyclization (e.g. an acetyl group or biotin) attached and C₂ is a cysteine residue and optionally has a moiety to promote cyclization (e.g. an NH₂ moiety) attached.

Soluble OMgp polypeptides described herein may have various alterations such as substitutions, insertions or deletions. Corresponding fragments of soluble OMgp polypeptides at least 70%, 75%, 80%, 85%, 90%, or 95% identical to polypeptides of SEQ ID NO:2 described herein are also contemplated.

As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

Soluble OMgp polypeptides for use in the methods of the present invention may include any combination of two or more soluble OMgp polypeptides.

### Antibodies or Immunospecific or Antigen-Binding Fragments Thereof

OMgp antagonists for use in the methods of the present invention also include OMgp-specific antibodies or antigen-binding fragments, variants, or derivatives which are antagonists of OMgp activity. For example, binding of certain OMgp antibodies to OMgp, as expressed on oligodendrocytes or myelin, block inhibition of neuron or oligodendrocyte survival, proliferation and differentiation, or block demyelination or dysmyelination of CNS neurons.

Antagonist antibodies, or fragments thereof, for use in the methods described herein specifically or preferentially bind to an OMgp polypeptide comprising one or more OMgp domains such as described in Table 1. For example, a polypeptide comprising an OMgp cysteine-rich domain, LRR domain or serine/threonine-rich domain. Antibodies of the invention also specifically or preferentially bind to OMgp polypeptides which have multiple OMgp domains fused or linked by peptide linkers.

Certain antagonist antibodies for use in the methods described herein specifically or preferentially binds to a particular epitope or eptiopes within an OMgp polypeptide fragment or domain (e.g. a domain as described in Table 1). Such OMgp polypeptide fragments include, but are not limited to, an OMgp polypeptide comprising, consisting essentially of, or consisting of amino acids amino acids 1 to 56 of SEQ ID NO:2; amino acids 1 to 228 of SEQ ID NO:2; amino acids 1 to 425 of SEQ ID NO:2; amino acids 1 to 440 of SEQ ID NO:2; amino acids 25 to 56 of SEQ ID NO:2; amino acids 25 to 228 of SEQ ID NO:2; amino acids 25 to 425 of SEQ ID NO:2; amino acids 25 to 440 of SEQ ID NO:2; amino acids 57 to 228 of SEQ ID NO:2; amino acids 229 to 425 of SEQ ID NO:2; amino acids 425 to 440 of SEQ ID NO:2; amino acids 57 to 425 of SEQ ID NO:2; amino acids 229 to 440 of SEQ ID NO:2. Corresponding fragments of a variant OMgp polypeptide at least 70%, 75%, 80%, 85%, 90%, or 95% identical to amino acids or derivatives of any of said polypeptide fragments are also contemplated.

In other embodiments, the present invention includes an antibody, or antigen-binding fragment, variant, or derivative thereof which specifically or preferentially binds to at least one epitope of OMgp, where the epitope comprises, consists essentially of, or consists of at least about four to five amino acids of SEQ ID NO:2, at least seven, at least nine, or between at least about 15 to about 30 amino acids of SEQ ID NO:2. The amino acids of a given epitope of SEQ ID NO:2 as described may be, but need not be contiguous or linear. In certain embodiments, the at least one epitope of OMgp comprises, consists essentially of, or consists of a non-linear epitope formed by OMgp as expressed on the surface of a cell or as a soluble fragment, e.g., fused to an IgG Fc region. Thus, in certain embodiments the at least one epitope of OMgp comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, where non-contiguous amino acids form an epitope through protein folding.

In other embodiments, the present invention includes an antibody, or antigen-binding fragment, variant, or derivative thereof which specifically or preferentially binds to at least one epitope of OMgp, where the epitope comprises, consists essentially of, or consists of, in addition to one, two, three, four, five, six or more contiguous or non-contiguous amino acids of SEQ ID NO:2 as described above, and an additional moiety which modifies the protein, e.g., a carbohydrate moiety may be included such that the OMgp antibody binds with higher affinity to modified target protein than it does to an unmodified version of the protein. Alternatively, the OMgp antibody does not bind the unmodified version of the target protein at all.

In certain embodiments, an antibody, or antigen-binding fragment, variant, or derivative thereof of the invention binds specifically to at least one epitope of OMgp or fragment or variant described above, i.e., binds to such an epitope more readily than it would bind to an unrelated, or random epitope; binds preferentially to at least one epitope of OMgp or fragment or variant described above, i.e., binds to such an epitope more readily than it would bind to a related, similar, homologous, or analogous epitope; competitively inhibits binding of a reference antibody which itself binds specifically or preferentially to a certain epitope of OMgp or fragment or variant described above; or binds to at least one epitope of OMgp or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷M, about 10⁻⁷ M, about 5×10⁻⁸ M, about 10⁻⁸ M, about 5×10⁻⁹ M, about 10⁻⁹M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. In a particular aspect, the antibody or fragment thereof preferentially binds to a human OMgp polypeptide or fragment thereof, relative to a murine OMgp polypeptide or fragment thereof.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, variant, or derivative thereof of the invention binds OMgp polypeptides or fragments or variants thereof with an off rate (k(_{off})) of less than or equal to 5×10⁻² sec⁻¹, 10⁻² sec, 5×10⁻³ sec⁻¹ or 10⁻³ sec⁻¹. Alternatively, an antibody, or antigen-binding fragment, variant, or derivative thereof of the invention binds OMgp polypeptides or fragments or variants thereof with an off rate (k(_{off})) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, an antibody, or antigen-binding fragment, variant, or derivative thereof of the invention binds OMgp polypeptides or fragments or variants thereof with an on rate (k(ₒₙ)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an antibody, or antigen-binding fragment, variant, or derivative thereof of the invention binds OMgp polypeptides or fragments or variants thereof with an on rate (k(ₒₙ) greater than or equal to 10⁵ M⁻¹ sec⁻¹, 5×10⁶ M⁻¹ sec⁻¹, 10⁶ M⁻¹ sec⁻¹, or 5×10⁻⁶ M⁻¹ sec⁻¹ or 10⁷ M⁻¹ sec⁻¹.

In one embodiment, a OMgp antagonist for use in the methods of the invention is an antibody molecule, or immunospecific or antigen-binding fragment thereof. Unless it is specifically noted, as used herein a “fragment thereof” in reference to an antibody refers to an immunospecific or antigen-binding fragment, i.e., an antigen-specific or antigen-binding fragment. In one embodiment, an antibody of the invention is a bispecific binding molecule, binding polypeptide, or antibody, e.g., a bispecific antibody, minibody, domain deleted antibody, or fusion protein having binding specificity for more than one epitope, e.g., more than one antigen or more than one epitope on the same antigen. In one embodiment, a bispecific antibody has at least one binding domain specific for at least one epitope on OMgp. A bispecific antibody may be a tetravalent antibody that has two target binding domains specific for an epitope of OMgp and two target binding domains specific for a second target. Thus, a tetravalent bispecific antibody may be bivalent for each specificity.

In certain embodiments of the present invention comprise administration of an OMgp antagonist antibody, or immunospecific or antigen-binding fragment thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

### Polynucleotides Encoding OMgp Antibodies

The present invention also provides for nucleic acid molecules encoding OMgp antibodies, or antigen-binding fragments, variants, or derivatives thereof for use in the methods of the present invention.

In one embodiment, the present invention provides for the use of an isolated polynucleotide comprising, consisting essentially of, or consisting of a nucleic acid encoding an immunoglobulin heavy chain variable region (VH), where at least one of the CDRs of the heavy chain variable region or at least two of the VH-CDRs of the heavy chain variable region are at least 80%, 85%, 90% or 95% identical to reference heavy chain VH-CDR1, VH-CDR2, or VH-CDR3 amino acid sequences from monoclonal OMgp antibodies disclosed herein. Alternatively, the VH-CDR1, VH-CDR2, and VH-CDR3 regions of the VH are at least 80%, 85%, 90% or 95% identical to reference heavy chain VH-CDR1, VH-CDR2, and VH-CDR3 amino acid sequences from monoclonal OMgp antibodies disclosed herein. Thus, according to this embodiment a heavy chain variable region of the invention has VH-CDR1, VH-CDR2, or VH-CDR3 polypeptide sequences related to the polypeptide sequences shown in Table 2:

TABLE 2

Reference VH-CDR1, VH-CDR2, and VH-CDR3 amino acid
sequences*

| | VH SEQUENCE PN/PP (VH- | | | |
|---|---|---|---|---|
| | CDR1, VH-CDR2, and VH- | VH | VH | VH |
| Antibody | CDR3 underlined) | CDR1 | CDR2 | CDR3 |
| 35-E08 | GAAGTTCAATTGTTAGAGTCTGGTGGC | YYDMG | WISPSG | EPGEYC |
| | GGTCTTGTTCAGCCTGGTGGTTCTTTA | (SEQ ID | GKTVYA | SGGSCY |
| | CGTCTTTCTTGCGCTGCTTCCGGATTC | NO:33) | DSVKG | PDYGMD |
| | ACTTTCTCTTATTACGATATGGGTTGG | | (SEQ ID | V |
| | GTTCGCCAAGCTCCTGGTAAAGGTTTG | | NO:34) | (SEQ ID |
| | GAGTGGGTTTCTTGGATCTCTCCTTCT | | | NO:35) |
| | GGTGGCAAGACTGTTTATGCTGACTCC | | | |
| | GTTAAAGGTCGCTTCACTATCTCTAGA | | | |
| | GACAACTCTAAGAATACTCTCTACTTG | | | |
| | CAGATGAACAGCTTAAGGGCTGAGGAC | | | |
| | ACGGCCGTGTATTACTGTGCGAGAGAG | | | |
| | CCAGGGGAATATTGTAGTGGTGGTAGC | | | |
| | TGCTACCCGGACTACGGTATGGACGTC | | | |
| | TGGGGCCAAGGGACCACGGTCACCGTC | | | |
| | TCAAGC | | | |
| | (SEQ ID NO:16) | | | |
| | EVQLLESGGGLVQPGGSLRLSCAASGF | | | |
| | TFSYYDMGWVRQAPGKGLEWVSWISPS | | | |
| | GGKTVYADSVKGRFTISRDNSKNTLYL | | | |
| | QMNSLRAEDTAVYYCAREPGEYCSGGS | | | |
| | CYPDYGMDVWGQGTTVTVSS | | | |
| | (SEQ ID NO:37) | | | |
| 36-A08 | GAAGTTCAATTGTTAGAGTCTGGTGGC | AYWMD | VIGPSG | DLMGYS |
| | GGTCTTGTTCAGCCTGGTGGTTCTTTA | (SEQ ID | GFTDYA | YGSGY |
| | CGTCTTTCTTGCGCTGCTTCCGGATTC | NO:41) | DSVKG | (SEQ ID |
| | ACTTTCTCTGCTTACTGGATGGATTGG | | (SEQ ID | NO:43) |
| | GTTCGCCAAGCTCCTGGTAAAGGTTTG | | NO:42) | |
| | GAGTGGGTTTCTGTTATCGGTCCTTCT | | | |
| | GGTGGCTTTACTGATTATGCTGACTCC | | | |
| | GTTAAAGGTCGCTTCACTATCTCTAGA | | | |
| | GACAACTCTAAGAATACTCTCTACTTG | | | |
| | CAGATGAACAGCTTAAGGGCTGAGGAC | | | |
| | ACGGCCGTGTATTACTGTGCGAAAGAT | | | |
| | CTCATGGGATACAGCTATGGTTCTGGC | | | |
| | TACTGGGGCCAGGGCACCCTGGTCACC | | | |
| | GTCTCAAGC | | | |
| | (SEQ ID NO:18) | | | |
| | EVQLLESGGGLVQPGGSLRLSCAASGF | | | |
| | TFSAYWMDWVRQAPGKGLEWVSVIGPS | | | |
| | GGFTDYADSVKGRFTISRDNSKNTLYL | | | |
| | QMNSLRAEDTAVYYCAKDLMGYSYGSG | | | |
| | YWGQGTLVTVSS | | | |
| | (SEQ ID NO:45) | | | |
| 36-H06 | GAAGTTCAATTGTTAGAGTCTGGTGGC | LYDMD | SISSSG | RLDYDF |
| | GGTCTTGTTCAGCCTGGTGGTTGTTTA | (SEQ ID | GGTSYA | WRAGA |
| | CGTCTTTCTTGCGCTGCTTCCGGATTC | NO:49) | DSVKG | FDI |
| | ACTTTCTCTCTTTACGATATGGATTGG | | (SEQ ID | (SEQ ID |
| | GTTCGCCAAGCTCCTGGTAAAGGTTTG | | NO:50) | NO:51) |
| | GAGTGGGTTTCTTCTATCTCTTCTTCT | | | |
| | GGTGGCGGTACTTCTTATGCTGACTCC | | | |
| | GTTAAAGGTCGCTTCACTATCTCTAGA | | | |
| | GACAACTCTAAGAATACTCTCTACTTG | | | |
| | CAGATGAACAGCTTAAGGGCTGAGGAC | | | |
| | ACAGCCACATATTACTGTGCGAGACGT | | | |
| | CTGGATTACGATTTTTGGAGGGCGGGA | | | |
| | GCTTTTGATATCTGGGGCCAAGGGACA | | | |
| | ATGGTCACCGTCTCAAGC | | | |
| | (SEQ ID NO:20) | | | |
| | EVQLLESGGGLVQPGGSLRLSCAASGF | | | |
| | TFSLYDMDWVRQAPGKGLEWVSSISSS | | | |
| | GGGTSYADSVKGRFTISRDNSKNTLYL | | | |
| | QMNSLRAEDTATYYCARRLDYDFWRAG | | | |
| | AFDIWGQGTMVTVSS | | | |
| | (SEQ ID NO:53) | | | |
| 38-B02 | GAAGTTCAATTGTTAGAGTCTGGTGGC | VYDMM | SIWSSG | HAGGSY |
| | GGTCTTGTTCAGCCTGGTGGTTCTTTA | (SEQ ID | GGTYYA | QNDAFD |
| | CGTCTTTCTTGCGCTGCTTCCGGATTC | NO:57) | DSVKG | I |
| | ACTTTCTCTGTTTACGATATGATGTGG | | (SEQ ID | (SEQ ID |
| | GTTCGCCAAGCTCCTGGTAAAGGTTTG | | NO:58) | NO:59) |
| | GAGTGGGTTTCTTCTATCTGGTCTTCT | | | |
| | GGTGGCGGTACTTATTATGCTGACTCC | | | |
| | GTTAAAGGTCGCTTCACTATCTCTAGA | | | |
| | GACAACTCTAAGAATACTCTCTACTTG | | | |
| | CAGATGAACAGCTTAAGGGCTGAGGAC | | | |
| | ACAGCCGTGTATTACTGTACGAGACAT | | | |
| | GCTGGTGGGAGCTACCAAAATGATGCT | | | |
| | TTTGATATCTGGGGCCAAGGGACAATG | | | |
| | GTCACCGTCTCAAGC | | | |
| | (SEQ ID NO:22) | | | |
| | EVQLLESGGGLVQPGGSLRLSCAASGF | | | |
| | TFSVYDMMWVRQALPGKGLEWVSSIWS | | | |
| | SGGGTYYADSVKGRFTISRDNSKNTLY | | | |
| | LQMNSLRAEDTAVYYCTRHAGGSYQND | | | |
| | AFDIWGQGTMVTVSS | | | |
| | (SEQ ID NO:61) | | | |
| 38-F11 | GAAGTTCAATTGTTAGAGTCTGGTGGC | IYAME | SISPSG | TINYYY |
| | GGTCTTGTTCAGCCTGGTGGTTCTTTA | (SEQ ID | GWTEYA | DSSGYV |
| | CGTCTTTCTTGCGCTGCTTCCGGATTC | NO:65) | DSVKG | GDY |
| | ACTTTCTCTATTTACGCTATGGAGTGG | | (SEQ ID | (SEQ ID |
| | GTTCGCCAAGCTCCTGGTAAAGGTTTG | | NO:66) | NO:67) |
| | GAGTGGGTTTCTTCTATCTCTCCTTCT | | | |
| | GGTGGCTGGACTGAGTATGCTGACTCC | | | |
| | GTTAAAGGTCGCTTCACTATCTCTAGA | | | |
| | GACAACTCTAAGAATACTCTCTACTTG | | | |
| | CAGATGAACAGCTTAAGGGCTGAGGAC | | | |
| | ACGGCCGTGTATTACTGTGCGAGAACC | | | |
| | ATTAATTATTACTATGATAGTAGTGGT | | | |
| | TATGTAGGGGACTACTGGGGCCAGGGA | | | |
| | ACCCTGGTCACCGTCTCAAGC | | | |
| | (SEQ ID NO:24) | | | |
| | EVQLLESGGGLVQPGGSLRLSCAASGF | | | |
| | TFSIYAMEWVRQAPGKGLEWVSSISPS | | | |
| | GGWTEYADSVKGRFTISRDNSKNTLYL | | | |
| | QMNSLRAEDTAVYYCARTINYYYDSSG | | | |
| | YVGDYWGQGTLVTVSS | | | |
| | (SEQ ID NO:69) | | | |
| 38-H02 | GAAGTTCAATTGTTAGAGTCTGGTGGC | FYDMV | RIYPSG | ASQYYY |
| | GGTCTTGTTCAGCCTGGTGGTTCTTAC | (SEQ ID | GPTSYA | GSGSYA |
| | GTCTTTCTTGCGCTGCTTCCGGATTCA | NO:73) | DSVKG | FDI |
| | CTTTCTTTTTACGATATGGTTTGGGTT | | (SEQ ID | (SEQ ID |
| | CGCCAAGCTCCTGGTAAAGGTTTGGAG | | NO:74) | NO:75) |
| | TGCCAAGCTCCTGGTAAAGGTTTGGAG | | | |
| | TGGGTTTCTCGTATCTATCCTTCTGGT | | | |
| | GGCCCTACTTCTTATGCTGACTCCGTT | | | |
| | AAAGGTCGCTTCACTATCTCTAGAGAC | | | |
| | AACTCTAAGAATACTCTCTACTTGCAG | | | |
| | ATGAACAGCTTAAGGGCTGAGGACACG | | | |
| | GCCGTGTATTACTGTGCGAGGGCCAGC | | | |
| | CAGTATTACTATGGTTCGGGGAGTTAT | | | |
| | GCTTTTGATATCTGGGGCCAAGGGACA | | | |
| | ATGGTCACCGTCTCAAGC | | | |
| | (SEQ ID NO:26) | | | |
| | EVQLLESGGGLVQPGGSLRLSCAASGF | | | |
| | TFSFYDMVWVRQAPGKGLEWVSRIYPS | | | |
| | GGPTSYADSVKGRFTISRDNSKNTLYL | | | |
| | QMNSLRAEDTAVYYCARASQYYYGSGS | | | |
| | YAFDIWGQGTMVTVSS | | | |
| | (SEQ ID NO:77) | | | |
| 38-H11 | EVQLLESGGGLVQPGGSLRISCAASGF | FYFMD | VISPSG | DLVYGD |
| | TFSFYFMDWVRQALPGKGLEWVSVISP | (SEQ ID | GSTFYA | HGGY |
| | SGGSTFYADSVKGRFTISRDNSKNTLY | NO:81) | DSVKG | (SEQ ID |
| | LQMNSLRAEDTAVYYCARDLVYGDHGG | | (SEQ ID | NO:83 |
| | YWGQGTLVTVSS | | NO:82) | |
| | (SEQ ID NO:85) | | | |
| 39-F09 | GAAGTTCAATTGTTAGAGTCTGGTGGC | LYEMG | VIGPSG | GASWGF |
| | GGTCTTGTTCAGCCTGGTGGTTCTTTA | (SEQ ID | GETFYA | HTEEYA |
| | CGTCTTTCTTGCGCTGCTTCCGGATTC | NO:89) | DSVKG | FDI |
| | ACTTTCTCTCTTTACGAGATGGGTTGG | | (SEQ ID | (SEQ ID |
| | GTTCGCCAAGCTCCTGGTAAAGGTTTG | | NO:90) | NO:91) |
| | GAGTGGGTTTCTGTTATCGGTCCTTCT | | | |
| | GGTGGCGAGACTTTTTATGCTGACTCC | | | |
| | GTTAAAGGTCGCTTCACTATCTCTAGA | | | |
| | GACAACTCTAAGAATACTCTCTACTTG | | | |
| | CAGATGAACAGCTTAAGGGCTGAGGAC | | | |
| | ACGGCCGTGTATTACTGTGCGAGAGGG | | | |
| | GCGTCCTGGGGGTTCCATACCGAGGAA | | | |
| | TATGCTTTTGATATCTGGGGCCAAGGG | | | |
| | ACAATGGTCACCGTCTCAAGC | | | |
| | (SEQ ID NO:28) | | | |
| | EVQLLESGGGLVQPGGSLRLSCAASGF | | | |
| | TFSLYEMGWVRQAPGKGLEWVSVIGPS | | | |
| | GGETFYADSVKGRFTISRDNSKNTLYL | | | |
| | QMNSLRAEDTAVYYCARGASWGFHTEE | | | |
| | YAFDIWGQGTMVTVSS | | | |
| | (SEQ ID NO:93) | | | |

*Detemined by the Kabat system (see supra).
N = nucleotide sequence, P = polypeptide sequence.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In certain embodiments, an antibody or antigen-binding fragment comprising the VH encoded by the polynucleotide specifically or preferentially binds to OMgp. In certain embodiments the nucleotide sequence encoding the VH polypeptide is altered without altering the amino acid sequence encoded thereby. For instance, the sequence may be altered for improved codon usage in a given species, to remove splice sites, or the remove restriction enzyme sites. Sequence optimizations such as these are described in the examples and are well known and routinely carried out by those of ordinary skill in the art.

In another embodiment, the present invention provides for the use of an isolated polynucleotide comprising, consisting essentially of, or consisting of a nucleic acid encoding an immunoglobulin heavy chain variable region (VH) in which the VH-CDR1, VH-CDR2, and VH-CDR3 regions have polypeptide sequences which are identical to the VH-CDR1, VH-CDR2, and VH-CDR3 groups shown in Table 2. In certain embodiments, an antibody or antigen-binding fragment comprising the VH encoded by the polynucleotide specifically or preferentially binds to OMgp.

In certain embodiments, an antibody or antigen-binding fragment thereof comprising, consisting essentially of, or consisting of a VH encoded by one or more of the polynucleotides described above specifically or preferentially binds to the same OMgp epitope as a reference monoclonal antibody fragment selected from the group consisting of 35-E08, 36-A08, 36-H06, 38-B02, 38-F11, 38-H02, 38-H11, and 39-F09, or will competitively inhibit such a monoclonal antibody or fragment from binding to OMgp.

In certain embodiments, an antibody or antigen-binding fragment thereof comprising, consisting essentially of, or consisting of a VH encoded by one or more of the polynucleotides described above specifically or preferentially binds to an OMgp polypeptide or fragment thereof, or a OMgp variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹⁴ M, 5×10⁻¹⁴ M, 10⁻¹⁵ M, 5×10⁻¹⁵ M, or 10⁻¹⁵ M.

In another embodiment, the present invention provides for the use of an isolated polynucleotide comprising, consisting essentially of, or consisting of a nucleic acid encoding an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2, or VL-CDR3 amino acid sequences from monoclonal OMgp antibodies disclosed herein. Alternatively, the VL-CDR1, VL-CDR2, and VL-CDR3 regions of the VL are at least 80%, 85%, 90% or 95% identical to reference light chain VL-CDR1, VL-CDR2, and VL-CDR3 amino acid sequences from monoclonal OMgp antibodies disclosed herein. Thus, according to this embodiment a light chain variable region of the invention has VL-CDR1, VL-CDR2, or VL-CDR3 polypeptide sequences related to the polypeptide sequences shown in Table 3:

TABLE 3

Reference VL-CDR1, VL-CDR2, and VL-CDR3 amino acid
sequences*

| | VL SEQUENCE PN/PP (VL- | | | |
|---|---|---|---|---|
| | CDR1, VL-CDR2, and VL- | VL | VL | VL |
| Antibody | CDR3 underlined) | CDR1 | CDR2 | CDR3 |
| 35-E08 | GACATCCAAATGACCCAGTCTCCATCCT | RASQSIS | AASSLQS | QQSYTT |
| | CCCTGTCTGCATCTGTAGGAGACAGAGT | SYLN | (SEQ ID | PRT |
| | CACCATCACTTGCCGGGCAAGTCAGAGC | (SEQ ID | NO:31) | (SEQ ID |
| | ATTAGCAGCTATTTAAATTGGTATCAGC | NO:30) | | NO:32) |
| | AGAAACCAGGGAAAGCCCCTAAGCTCCT | | | |
| | GATCTATGCTGCATCCAGTTTGCAAAGT | | | |
| | GGGGTCCCATCAAGGTTCAGTGGCAGTG | | | |
| | GGTCTGGGACAGATTTCACTCTCACCAT | | | |
| | CAGCAGTCTGCAACCTGAAGATTTTGCA | | | |
| | ACTTACTACTGTCAACAGAGTTACACTA | | | |
| | CCCCTCGAACGTTCGGCCAAGGGACCAA | | | |
| | GGTGGACATCAGA | | | |
| | (SEQ ID NO:17) | | | |
| | DIQMTQSPSSLSASVGDRVTITCRASQS | | | |
| | ISSYLNWYQQKPGKAPKLLIYAASSLQS | | | |
| | GVPSRFSGSGSGTDFTLTISSLQPEDFA | | | |
| | TYYCQQSYTTPRTFGQGTKVDIR | | | |
| | (SEQ ID NO:36) | | | |
| 36-A08 | CAGAGCGAATTGACTCAGCCTGCCTCCG | TGTSSDV | EVSNRPS | SSYTSS |
| | TGTCTGGGTCTCCTGGACAGGCGATCAC | GGYNYVS | (SEQ ID | STWV |
| | CATCTCCTGCACTGGAACCAGCAGTGAC | (SEQ ID | NO:39) | (SEQ ID |
| | GTTGGTGGTTATAACTATGTCTCCTGGT | NO:38) | | NO:40) |
| | ACCAACAGCACCCAGGCAAAGCCCCCAA | | | |
| | ACTCATGATTTATGAGGTCAGTAATCGG | | | |
| | CCCTCAGGGGTTTCAAATCGCTTCTCTG | | | |
| | GCTCCAAGTCTGGCAACACGGCCTCCCT | | | |
| | GACCATCTCTGGGCTCCAGGCTGAGGAC | | | |
| | GAGGCTGATTATTACTGCAGCTCATATA | | | |
| | CAAGCAGCAGCACATGGGTGTTCGGCGG | | | |
| | AGGGACCAAACTGACCGTCCTG | | | |
| | (SEQ ID NO:19) | | | |
| | QSELTQPASVSGSPGQAITISCTGTSSD | | | |
| | VGGYNYVSWYQQHPGKAPKLMIYEVSNR | | | |
| | PSGVSNRFSGSKSGNTASLTISGLQAED | | | |
| | EADYYCSSYTSSSTWVFGGGTKLTVL | | | |
| | (SEQ ID NO:44) | | | |
| 36-H06 | GACATCCAGATGACCCAGTCTCCATCCT | RASQSIS | AASSLQS | QQSYST |
| | CCCTGTCTGCATCTGTAGGAGACAGAGT | SYLN | (SEQ ID | PRT |
| | CACCATCACTTGCCGGGCAAGTCAGAGC | (SEQ ID | NO:47) | (SEQ ID |
| | ATTAGCAGCTATTTAAATTGGTATCAGC | NO:46) | | NO:48) |
| | AGAAACCAGGGAAAGCCCCTAAGCTCCT | | | |
| | GATCTATGCTGCATCCAGTTTGCAAAGT | | | |
| | GGGGTCCCATCAAGGTTCAGTGGCAGTG | | | |
| | GATCTGGGACAGATTTCACTCTCACCAT | | | |
| | CAGCAGTCTGCAACCTGAAGATTTTGCA | | | |
| | ACTTACTACTGTCAACAGAGTTACAGTA | | | |
| | CCCCTCGAACGTTCGGCCAAGGGACCAA | | | |
| | GGTGGAAATCAAA | | | |
| | (SEQ ID NO:21) | | | |
| | DIQMTQSPSSLSASVGDRVTITCRASQS | | | |
| | ISSYLNWYQQKPGKAPKLLIYAASSLQS | | | |
| | GVPSRFSGSGSGTDFTLTISSLQPEDFA | | | |
| | TYYCQQSYSTPRTFGQGTKVEIK | | | |
| | (SEQ ID NO:52) | | | |
| 38-B02 | GACATCCAGATGACCCAGTCTCCATCTT | RASQGIN | AASSLQD | QQGNSF |
| | CCGTGTCTGCATCTGTGGGAGACAGAGT | SWLA | (SEQ ID | PPT |
| | CACCATCACTTGTCGGGCGAGTCAAGGT | (SEQ ID | NO:55) | (SEQ ID |
| | ATTAACAGCTGGTTAGCCTGGTATCAGC | NO:54) | | NO:56) |
| | AGAAACCTGGGACAGCCCCTAAACTCCT | | | |
| | GATCTCTGCTGCATCCAGTTTGCAAGAT | | | |
| | GGGGTCCCATCAAGGTTCAGCGGCGGTG | | | |
| | GATCTGGGACAGAGTTCACTCTCACCAT | | | |
| | CAGCAGCCTGCAGCCTGAAGATTTTGCA | | | |
| | ACTTACTATTGTCAACAGGGTAACAGTT | | | |
| | TCCCTCCGACCTTCGGCGGAGGGACCAG | | | |
| | AGTAGAGATCAGA | | | |
| | (SEQ ID NO:23) | | | |
| | DIQMTQSPSSVSASVGDRVTITCRASQG | | | |
| | INSWLAWYQQKPGTAPKLLISAASSLQD | | | |
| | GVPSRFSGGGSGTEFTLTISSLQPEDFA | | | |
| | TYYCQQGNSFPPTFGGGTRVEIR | | | |
| | (SEQ ID NO:60) | | | |
| 33-F11 | GACATCCAGATGACCCAGTCTCCTTCCA | RASQNIS | EASNLET | QQYQTY |
| | CCCTGTCTGCCTCTGTAGGAGACAGAGT | TWLA | (SEQ ID | SWT |
| | CACCATGACTTGCCGGGCCAGTCAGAAT | (SEQ ID | NO:63) | (SEQ ID |
| | ATTAGTACCTGGTTGGCCTGGTATCAGC | NO:62) | | NO:64) |
| | AGAAACCAGGGAAAGCCCCTAAACTCCT | | | |
| | GATCTATGAGGCATCTAATTTAGAAACT | | | |
| | GGGGTCCCATCAAGGTTGAGCGGCAGTG | | | |
| | GATCTGGGACAGAATTCACTCTCACCAT | | | |
| | CGCCAGCCTGCAGCCTGATGATTTTGCA | | | |
| | GCTTATTACTGCCAACAGTATCAGACTT | | | |
| | ATTCGTGGACGTTCGGCCAAGGGACCAA | | | |
| | GGTGGATGTCAAA | | | |
| | (SEQ ID NO:25) | | | |
| | DIQMTQSPSTLSASVGDRVTMTCRASQN | | | |
| | ISTWLAWYQQKPGKAPKLLIYEASNLET | | | |
| | GVPSRFSGSGSGTEFTLTIASLQPDDFA | | | |
| | AYYCQQYQTYSWTFGQGTKVDVK | | | |
| | (SEQ ID NO:68) | | | |
| 38-H02 | GACATCCAGATGACCCAGTCTCCATCCT | RASQSIS | AASSLQS | QQSYST |
| | CCCTGTCTGCATCTGTAGGAGACAGAGT | SYLN | (SEQ ID | PWT |
| | CACCATCACTTGCCGGGCAAGTCAGAGC | (SEQ ID | NO:71) | (SEQ ID |
| | ATTAGCAGCTATTTAAATTGGTATCAGC | NO:70) | | NO:72) |
| | AGAAACCAGGGAAAGCCCCTAAGCTCCT | | | |
| | GATCTATGCTGCATCCAGTTTGCAAAGT | | | |
| | GGGGTCCCATCAAGGTTCAGTGGCAGTG | | | |
| | GATCTGGGACAGATTTCACTCTCACCAT | | | |
| | CAGCAGTCTGCAACCTGAAGATTTTGCA | | | |
| | ACTTACTACTGTCAACAGAGTTACAGTA | | | |
| | CCCCTTGGACGTTCGGCCAAGGGACCAA | | | |
| | GGTGGAAATCAAA | | | |
| | (SEQ ID NO:27) | | | |
| | DIQMTQSPSSLSASVGDRVTITCRASQS | | | |
| | ISSYLNWYQQKPGKAPKLLIYAASSLQS | | | |
| | GVPSRFSGSGSGTDFTLTISSLQPEDFA | | | |
| | TYYCQQSYSTPWTFGQGTKVEIK | | | |
| | (SEQ ID NO:76) | | | |
| 38-H11 | QYELTQPASVSGSPGQSITISCTGTSSD | TGTSSDV | DVSNRPS | SSYTSS |
| | VGGYNYVSWYQQHPGKAPKLMIYDVSNR | GGYNYVS | (SEQ ID | STYV |
| | PSGVSNRFSGSKSGNTASLTISGLQAED | (SEQ ID | NO:79) | (SEQ ID |
| | EADYYCSSYTSSSTYVFGTGTKVTVL | NO:78) | | NO:80) |
| | (SEQ ID NO:84) | | | |
| 39-F09 | GACATCCAGATGACCCAGTCTCCATCCT | RASQSIS | AASSLQS | QQSFSS |
| | CCCTGTCTGCATCTGTAGGAGACAGAGT | SYLN | (SEQ ID | PST |
| | CACCATCACTTGCCGGGCAAGTCAGAGC | (SEQ ID | NO:87) | (SEQ ID |
| | ATTAGCAGCTATTTAAATTGGTATCAGC | NO:86) | | NO:88) |
| | AGAAACCAGGGAAAGCCCCTAAGCTCCT | | | |
| | GATCTATGCTGCATCCAGTTTGCAAAGT | | | |
| | GGGGTCCCATCAAGGTTCAGTGCCACTG | | | |
| | GATCTGGGACAGACTTCACTCTCTCCAT | | | |
| | CAGCAGTCTGCAACCTGAAGATTTTGCA | | | |
| | ACTTACTACTGTCAACAGAGTTTCAGTT | | | |
| | CCCCTAGCACTTTTGGCCAGGGGACCAA | | | |
| | CCTGGAGATCAAA | | | |
| | (SEQ ID NO:29) | | | |
| | DIQMTQSPSSLSASVGDRVTITCRASQS | | | |
| | ISSYLNWYQQKPGKAPKLLIYAASSLQS | | | |
| | GVPSRFSATGSGTDFTLSISSLQPEDFA | | | |
| | TYYCQQSFSSPSTFGQGTNLEIK | | | |
| | (SEQ ID NO:92) | | | |

*Determined by the Kabat system (see supra).
PN = nucleotide sequence, PP = polypeptide sequence.

In certain embodiments, an antibody or antigen-binding fragment comprising the VL encoded by the polynucleotide specifically or preferentially binds to OMgp.

In another embodiment, the present invention provides for the use of an isolated polynucleotide comprising, consisting essentially of, or consisting of a nucleic acid encoding an immunoglobulin light chain variable region (VL) in which the VL-CDR1, VL-CDR2, and VL-CDR3 regions have polypeptide sequences which are identical to the VL-CDR1, VL-CDR2, and VL-CDR3 groups shown in Table 3. In certain embodiments, an antibody or antigen-binding fragment comprising the VL encoded by the polynucleotide specifically or preferentially binds to OMgp.

In a further aspect, the present invention provides for the use of an isolated polynucleotide comprising, consisting essentially of, or consisting of a nucleic acid encoding an immunoglobulin light chain variable region (VL) in which the VL-CDR1, VL-CDR2, and VL-CDR3 regions are encoded by nucleotide sequences which are identical to the nucleotide sequences which encode the VL-CDR1, VL-CDR2, and VL-CDR3 groups shown in Table 3. In certain embodiments, an antibody or antigen-binding fragment comprising the VL encoded by the polynucleotide specifically or preferentially binds to OMgp.

In certain embodiments, an antibody or antigen-binding fragment thereof comprising, consisting essentially of, or consisting of a VL encoded by one or more of the polynucleotides described above specifically or preferentially binds to the same OMgp epitope as a reference monoclonal antibody fragment selected from the group consisting of 35-E08, 36-A08, 36-H06, 38-B02, 38-F11, 38-H02, 38-H11, and 39-F09, or will competitively inhibit such a monoclonal antibody or fragment from binding to reference monoclonal antibody fragment selected from the group consisting of 35-E08, 36-A08, 36-H06, 38-B02, 38-F11, 38-H02, 38-H11, and 39-F09.

In certain embodiments, an antibody or antigen-binding fragment thereof comprising, consisting essentially of, or consisting of a VL encoded by one or more of the polynucleotides described above specifically or preferentially binds to an OMgp polypeptide or fragment thereof, or a OMgp variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹⁴ M, 5×10⁻¹⁴ M, 10⁻¹⁵ M, 5×10⁻¹⁵ M, or 10⁻¹⁵ M.

In a further embodiment, the present invention includes the use of an isolated polynucleotide comprising, consisting essentially of, or consisting of a nucleic acid encoding a VH at least 80%, 85%, 90% 95% or 100% identical to a reference VH polypeptide sequence selected from the group consisting of SEQ ID NOs: 37, 45, 53, 61, 69, 77, 85, and 93. In certain embodiments, an antibody or antigen-binding fragment comprising the VH encoded by the polynucleotide specifically or preferentially binds to OMgp.

In another aspect, the present invention includes the use of an isolated polynucleotide comprising, consisting essentially of, or consisting of a nucleic acid sequence encoding a VH having a polypeptide sequence selected from the group consisting of SEQ ID NOs: 37, 45, 53, 61, 69, 77, 85, and 93. In certain embodiments, an antibody or antigen-binding fragment comprising the VH encoded by the polynucleotide specifically or preferentially binds to OMgp.

In a further embodiment, the present invention includes the use of an isolated polynucleotide comprising, consisting essentially of, or consisting of a VH-encoding nucleic acid at least 80%, 85%, 90% 95% or 100% identical to a reference nucleic acid sequence selected from the group consisting of SEQ ID NOs: 16, 18, 20, 22, 24, 26, and 28. In certain embodiments, an antibody or antigen-binding fragment comprising the VH encoded by such polynucleotides specifically or preferentially binds to OMgp.

In another aspect, the present invention includes the use of an isolated polynucleotide comprising, consisting essentially of, or consisting of a nucleic acid sequence encoding a VH of the invention, where the amino acid sequence of the VH is selected from the group consisting of SEQ ID NOs: 37, 45, 53, 61, 69, 77, 85, and 93. The present invention further includes an isolated polynucleotide comprising, consisting essentially of, or consisting of a nucleic acid sequence encoding a VH of the invention, where the sequence of the nucleic acid is selected from the group consisting of SEQ ID NOs: 16, 18, 20, 22, 24, 26, and 28. In certain embodiments, an antibody or antigen-binding fragment comprising the VH encoded by such polynucleotides specifically or preferentially binds to OMgp.

In certain embodiments, an antibody or antigen-binding fragment thereof comprising, consisting essentially of, or consisting of a VH encoded by one or more of the polynucleotides described above specifically or preferentially binds to the same OMgp epitope as a reference monoclonal antibody fragment selected from the group consisting of 35-E08, 36-A08, 36-H06, 38-B02, 38-F11, 38-H02, 38-H11, and 39-F09, or will competitively inhibit such a monoclonal antibody or fragment from binding to OMgp, or will competitively inhibit such a monoclonal antibody from binding to OMgp.

In certain embodiments, an antibody or antigen-binding fragment thereof comprising, consisting essentially of, or consisting of a VH encoded by one or more of the polynucleotides described above specifically or preferentially binds to an OMgp polypeptide or fragment thereof, or a OMgp variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10¹⁴ M, 10⁻¹⁴ M, 5×10⁻¹⁵ M, or 10⁻¹⁵ M.

In a further embodiment, the present invention includes the use of an isolated polynucleotide comprising, consisting essentially of, or consisting of a nucleic acid encoding a VL at least 80%, 85%, 90% 95% or 100% identical to a reference VL polypeptide sequence having an amino acid sequence selected from the group consisting of SEQ ID NOs: 36, 44, 52, 60, 68, 76, 84, and 92. In a further embodiment, the present invention includes the use of an isolated polynucleotide comprising, consisting essentially of, or consisting of a VL-encoding nucleic acid at least 80%, 85%, 90% 95% or 100% identical to a reference nucleic acid sequence selected from the group consisting of SEQ ID NOs: 17, 19, 21, 23, 25, 27, and 29. In certain embodiments, an antibody or antigen-binding fragment comprising the VL encoded by such polynucleotides specifically or preferentially binds to OMgp.

In another aspect, the present invention includes the use of an isolated polynucleotide comprising, consisting essentially of, or consisting of a nucleic acid sequence encoding a VL having a polypeptide sequence selected from the group consisting of SEQ ID NOs: 36, 44, 52, 60, 68, 76, 84, and 92. The present invention further includes the use of an isolated polynucleotide comprising, consisting essentially of, or consisting of a nucleic acid sequence encoding a VL of the invention, where the sequence of the nucleic acid is selected from the group consisting of SEQ ID NOs: 17, 19, 21, 23, 25, 27, and 29. In certain embodiments, an antibody or antigen-binding fragment comprising the VL encoded by such polynucleotides specifically or preferentially binds to OMgp.

In certain embodiments, an antibody or antigen-binding fragment thereof comprising, consisting essentially of, or consisting of a VL encoded by one or more of the polynucleotides described above specifically or preferentially binds to the same OMgp epitope as a reference monoclonal antibody fragment selected from the group consisting of 35-E08, 36-A08, 36-H06, 38-B02, 38-F11, 38-H02, 38-H11, and 39-F09, or will competitively inhibit such a monoclonal antibody or fragment from binding to OMgp.

In certain embodiments, an antibody or antigen-binding fragment thereof comprising, consisting essentially of, or consisting of a VL encoded by one or more of the polynucleotides described above specifically or preferentially binds to an OMgp polypeptide or fragment thereof, or a OMgp variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above may further include additional nucleic acids, encoding, e.g., a signal peptide to direct secretion of the encoded polypeptide, antibody constant regions as described herein, or other heterologous polypeptides as described herein.

Also, as described in more detail elsewhere herein, the present invention includes compositions comprising the polynucleotides comprising one or more of the polynucleotides described above. In one embodiment, the invention includes compositions comprising a first polynucleotide and second polynucleotide wherein said first polynucleotide encodes a VH polypeptide as described herein and wherein said second polynucleotide encodes a VL polypeptide as described herein. Specifically a composition which comprises, consists essentially of, or consists of a VH polynucleotide, and a VL polynucleotide, wherein the VH polynucleotide and the VL polynucleotide encode polypeptides, respectively at least 80%, 85%, 90% 95% or 100% identical to reference VL and VH polypeptide amino acid sequences selected from the group consisting of SEQ ID NOs: 36 and 37, 44 and 45, 52 and 53, 60 and 61, 68 and 69, 76 and 77, 84 and 85, 92 and 93. Or alternatively, a composition which comprises, consists essentially of, or consists of a VH polynucleotide, and a VL polynucleotide at least 80%, 85%, 90% 95% or 100% identical, respectively, to reference VH and VL nucleic acid sequences selected from the group consisting of SEQ ID NOs: 16 and 17, 18 and 19, 20 and 21, 22 and 23, 24 and 25, 26 and 27, and 28 and 29. In certain embodiments, an antibody or antigen-binding fragment comprising the VH and VL encoded by the polynucleotides in such compositions specifically or preferentially binds to OMgp.

The present invention also includes fragments of the polynucleotides of the invention, as described elsewhere. Additionally polynucleotides which encode fusion polynucleotides, Fab fragments, and other derivatives, as described herein, are also contemplated by the invention.

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an OMgp antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other OMgp antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other OMgp antibody. Amplified nucleic acids generated by PCR may then be cloned into replicable cloning vectors using any method well known in the art.

Once the nucleotide sequence and corresponding amino acid sequence of the OMgp antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an OMgp antibody, or antigen-binding fragment, variant, or derivative thereof can be composed of any polyribonucleotide or polydeoxyribonucleotide, which may be unmodified RNA or DNA or modified RNA or DNA. For example, a polynucleotide encoding OMgp antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an OMgp antibody, or antigen-binding fragment, variant, or derivative thereof can be composed of triple-stranded regions comprising RNA or DNA or both RNA and DNA. A polynucleotide encoding an OMgp antibody, or antigen-binding fragment, variant, or derivative thereof may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

V. OMgp Antibody Polypeptides

The present invention is further directed to isolated polypeptides which make up OMgp antibodies, and polynucleotides encoding such polypeptides. OMgp antibodies of the present invention comprise polypeptides, e.g., amino acid sequences encoding OMgp-specific antigen binding regions derived from immunoglobulin molecules. A polypeptide or amino acid sequence “derived from” a designated protein refers to the origin of the polypeptide having a certain amino acid sequence. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where at least one of VH-CDRs of the heavy chain variable region or at least two of the VH-CDRs of the heavy chain variable region are at least 80%, 85%, 90% or 95% identical to reference heavy chain VH-CDR1, VH-CDR2 or VH-CDR3 amino acid sequences from monoclonal OMgp antibodies disclosed herein. Alternatively, the VH-CDR1, VH-CDR2 and VH-CDR3 regions of the VH are at least 80%, 85%, 90% or 95% identical to reference heavy chain VH-CDR1, VH-CDR2 and VH-CDR3 amino acid sequences from monoclonal OMgp antibodies disclosed herein. Thus, according to this embodiment a heavy chain variable region of the invention has VH-CDR1, VH-CDR2 and VH-CDR3 polypeptide sequences related to the groups shown in Table 2, supra. While Table 2 shows VH-CDRs defined by the Kabat system, other CDR definitions, e.g., VH-CDRs defined by the Chothia system, are also included in the present invention. In certain embodiments, an antibody or antigen-binding fragment comprising the VH specifically or preferentially binds to OMgp.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 2. In certain embodiments, an antibody or antigen-binding fragment comprising the VH specifically or preferentially binds to QMgp.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 2, except for one, two, three, four, five, or six amino acid substitutions in any one VH-CDR. In larger CDRs, e.g., VH-CDR-3, additional substitutions may be made in the CDR, as long as the a VH comprising the VH-CDR specifically or preferentially binds to OMgp. In certain embodiments the amino acid substitutions are conservative. In certain embodiments, an antibody or antigen-binding fragment comprising the VH specifically or preferentially binds to OMgp.

In a further embodiment, the present invention includes an isolated polypeptide comprising, consisting essentially of, or consisting of a VH polypeptide at least 80%, 85%, 90% 95% or 100% identical to a reference VH polypeptide amino acid sequence selected from the group consisting of SEQ ID NOs: SEQ ID NOs: 37, 45, 53, 61, 69, 77, 85, and 93. In certain embodiments, an antibody or antigen-binding fragment comprising the VH polypeptide specifically or preferentially binds to OMgp.

In another aspect, the present invention includes an isolated polypeptide comprising, consisting essentially of, or consisting of a VH polypeptide selected from the group consisting of SEQ ID NOs: 37, 45, 53, 61, 69, 77, 85, and 93. In certain embodiments, an antibody or antigen-binding fragment comprising the VH polypeptide specifically or preferentially binds to OMgp.

In certain embodiments, an antibody or antigen-binding fragment thereof comprising, consisting essentially of, or consisting of a one or more of the VH polypeptides described above specifically or preferentially binds to the same OMgp epitope as a reference monoclonal antibody fragment selected from the group consisting of 35-E08, 36-A08, 36-H06, 38-B02, 38-F11, 38-H02, 38-H11, and 39-F09, or will competitively inhibit such a monoclonal antibody or fragment from binding to OMgp.

In certain embodiments, an antibody or antigen-binding fragment thereof comprising, consisting essentially of, or consisting of one or more of the VH polypeptides described above specifically or preferentially binds to an OMgp polypeptide or fragment thereof, or a OMgp variant polypeptide, with an affinity characterized by a dissociation constant (K_{D}) no greater than 5×10⁻² M, 10⁻² M, 5×10⁻³M, 10⁻³ M, 5×10⁻⁴ M, 10⁻⁴M, 5×10⁻⁵M, 10⁻⁵M, 5×10⁻⁶M, 10⁻⁶M, 5×10⁻⁷M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting of an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2 or VL-CDR3 amino acid sequences from monoclonal OMgp antibodies disclosed herein. Alternatively, the VL-CDR1, VL-CDR2 and VL-CDR3 regions of the VL are at least 80%, 85%, 90% or 95% identical to reference light chain VL-CDR1, VL-CDR2 and VL-CDR3 amino acid sequences from monoclonal OMgp antibodies disclosed herein. Thus, according to this embodiment a light chain variable region of the invention has VL-CDR1, VL-CDR2 and VL-CDR3 polypeptide sequences related to the polypeptides shown in Table 3, supra. While Table 3 shows VL-CDRs defined by the Kabat system, other CDR definitions, e.g., VL-CDRs defined by the Chothia system, are also included in the present invention. In certain embodiments, an antibody or antigen-binding fragment comprising the VL polypeptide specifically or preferentially binds to OMgp.

In another embodiment, the present invention provides an isolated polypeptide comprising, consisting essentially of, or consisting of an immunoglobulin light chain variable region (VL) in which the VL-CDR1, VL-CDR2 and VL-CDR3 regions have polypeptide sequences which are identical to the VL-CDR1, VL-CDR2 and VL-CDR3 groups shown in Table 3. In certain embodiments, an antibody or antigen-binding fragment comprising the VL polypeptide specifically or preferentially binds to OMgp.

In another embodiment, the present invention provides an isolated polypeptide comprising, consisting essentially of, or consisting of an immunoglobulin heavy chain variable region (VL) in which the VL-CDR1, VL-CDR2 and VL-CDR3 regions have polypeptide sequences which are identical to the VL-CDR1, VL-CDR2 and VL-CDR3 groups shown in Table 3, except for one, two, three, four, five, or six amino acid substitutions in any one VL-CDR. In larger CDRs, additional substitutions may be made in the VL-CDR, as long as the a VL comprising the VL-CDR specifically or preferentially binds to OMgp. In certain embodiments the amino acid substitutions are conservative. In certain embodiments, an antibody or antigen-binding fragment comprising the VL specifically or preferentially binds to OMgp.

In a further embodiment, the present invention includes an isolated polypeptide comprising, consisting essentially of, or consisting of a VL polypeptide at least 80%, 85%, 90% 95% or 100% identical to a reference VL polypeptide sequence selected from the group consisting of SEQ ID NOs: 36, 44, 52, 60, 68, 76, 84, and 92. In certain embodiments, an antibody or antigen-binding fragment comprising the VL polypeptide specifically or preferentially binds to OMgp.

In another aspect, the present invention includes an isolated polypeptide comprising, consisting essentially of, or consisting of a VL polypeptide selected from the group consisting of SEQ ID NOs: 36, 44, 52, 60, 68, 76, 84, and 92. In certain embodiments, an antibody or antigen-binding fragment comprising the VL polypeptide specifically or preferentially binds to OMgp.

In certain embodiments, an antibody or antigen-binding fragment thereof comprising, consisting essentially of, one or more of the VL polypeptides described above specifically or preferentially binds to the same OMgp epitope as a reference monoclonal antibody fragment selected from the group consisting of 35-E08, 36-A08, 36-H06, 38-B02, 38-F11, 38-H02, 38-H11, and 39-F09, or will competitively inhibit such a monoclonal antibody or fragment from binding to OMgp.

In certain embodiments, an antibody or antigen-binding fragment thereof comprising, consisting essentially of, or consisting of a one or more of the VL polypeptides described above specifically or preferentially binds to an OMgp polypeptide or fragment thereof, or a OMgp variant polypeptide, with an affinity characterized by a dissociation constant (K_{D}) no greater than 5×10⁻² M, 10⁻² M, 5×10⁻³ M, 10³M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸M, 10⁻⁸M, 5×10⁻⁹M, 10⁻⁹M, 5×10⁻¹⁰M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In other embodiments, an antibody or antigen-binding fragment thereof comprises, consists essentially of or consists of a VH polypeptide, and a VL polypeptide, where the VH polypeptide and the VL polypeptide, respectively are at least 80%, 85%, 90% 95% or 100% identical to reference VL and VH polypeptide amino acid sequences selected from the group consisting of SEQ ID NOs: 36 and 37, 44 and 45, 52 and 53, 60 and 61, 68 and 69, 76 and 77, 84 and 85, 92 and 93. In certain embodiments, an antibody or antigen-binding fragment comprising these VH and VL polypeptides specifically or preferentially binds to OMgp.

Any of the polypeptides described above may further include additional polypeptides, e.g., a signal peptide to direct secretion of the encoded polypeptide, antibody constant regions as described herein, or other heterologous polypeptides as described herein. Additionally, polypeptides of the invention include polypeptide fragments as described elsewhere. Additionally polypeptides of the invention include fusion polypeptide, Fab fragments, and other derivatives, as described herein.

Also, as described in more detail elsewhere herein, the present invention includes compositions comprising the polypeptides described above.

It will also be understood by one of ordinary skill in the art that OMgp antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In other embodiments, a polypeptide or amino acid sequence derived from a designated protein may be identical to the starting sequence except for two or fewer, three or fewer, four or fewer, five or fewer, six or fewer, seven or fewer, eight or fewer, nine or fewer, ten or fewer, fifteen or fewer, or twenty or fewer individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain OMgp antibody polypeptides of the present invention comprise, consist essentially of, or consist of an amino acid sequence derived from a human amino acid sequence. However, certain OMgp antibody polypeptides comprise one or more contiguous amino acids derived from another mammalian species. For example, an OMgp antibody of the present invention may include a primate heavy chain portion, hinge portion, or antigen binding region. In another example, one or more murine-derived amino acids may be present in a non-murine antibody polypeptide, e.g., in an antigen binding site of an OMgp antibody. In another example, the antigen binding site of an OMgp antibody is fully murine. In certain therapeutic applications, OMgp-specific antibodies, or antigen-binding fragments, variants, or analogs thereof are designed so as to not be immunogenic in the animal to which the antibody is administered.

In certain embodiments, an OMgp antibody polypeptide comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

An OMgp antibody polypeptide for use in the method of the present invention may comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an OMgp antibody, or an antigen-binding fragment, variant, or analog thereof is derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into OMgp antibodies for use in the diagnostic and treatment methods disclosed herein and screened for their ability to bind to the desired antigen, e.g., OMgp.

In certain OMgp antagonist antibodies or immunospecific or antigen-binding fragments thereof for use in the therapeutic methods described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of antibodies or immunospecific or antigen-binding fragments thereof for use in the diagnostic and therapeutic methods disclosed herein can be made from whole precursor or parent antibodies using techniques known in the art. Exemplary techniques are discussed in more detail herein.

In certain embodiments both the variable and constant regions of OMgp antagonist antibodies or immunospecific or antigen-binding fragments thereof for use in the treatment methods disclosed herein are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human anti bodies can likewise be produced by various display technologies, e.g., phage display or other viral display systems, as described in more detail elsewhere herein.

OMgp antagonist antibodies or immunospecific or antigen-binding fragments thereof for use in the diagnostic and treatment methods disclosed herein can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

OMgp antagonist antibodies or immunospecific or antigen-binding fragments thereof for use in the treatment methods disclosed herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In preferred embodiments, an OMgp antagonist antibody or immunospecific or antigen-binding fragment thereof for use in the treatment methods disclosed herein will not elicit a deleterious immune response in the animal to be treated, e.g., in a human. In one embodiment, OMgp antagonist antibodies or immunospecific or antigen-binding fragments thereof for use in the treatment methods disclosed herein be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, V_{H} and V_{L} sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative V_{H} and V_{L} sequences are designed comprising combinations of amino acid substitutions and these sequences are subsequently incorporated into a range of binding polypeptides, e.g., OMgp antagonist antibodies or immunospecific or antigen-binding fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

OMgp antagonist antibodies or fragments thereof for use in the methods of the present invention may be generated by any suitable method known in the art. Polyclonal antibodies can be produced by various procedures well known in the art. For example, a OMgp immunospecific or antigen-binding fragment can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

Using art recognized protocols, in one example, antibodies are raised in mammals by multiple subcutaneous or intraperitoneal injections of the relevant antigen (e.g., purified OMgp antigens or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Antibody fragments that recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain.

Those skilled in the art will also appreciate that DNA encoding antibodies or antibody fragments (e.g., antigen binding sites) may also be derived from antibody phage libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead.

Phage used in these methods are typically filamentous phage including fd and M13 binding domains expressed from phage with Fab, Fv or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. In particular, DNA sequences encoding V_{H} and V_{L} regions are amplified from animal cDNA libraries (e.g., human or murine cDNA libraries of lymphoid tissues) or synthetic cDNA libraries. In certain embodiments, the DNA encoding the V_{H} and V_{L} regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} or V_{L} regions are usually recombinantly fused to either the phage gene III or gene VIII. Phage expressing an antigen binding domain that binds to an antigen of interest (i.e., a OMgp polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al, PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988)). See also, U.S. Pat. No. 5,565,332.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., OMgp. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E. coli may also be used (Skerra et al., Science 242:1038-1041 (1988)).

OMgp antagonist antibodies may also be human or substantially human antibodies generated in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the V_{H} and V_{L} genes can be amplified using, e.g., RT-PCR. The V_{H} and V_{L} genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies for use in the therapeutic methods disclosed herein can be produced by any method known in the art for the synthesis of antibodies, in particular, by chemical synthesis or preferably, by recombinant expression techniques as described herein.

It will further be appreciated that the scope of this invention further encompasses all alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Recombinant expression of an antibody, or fragment, derivative or analog thereof, e.g., a heavy or light chain of an antibody which is an OMgp antagonist, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharoinyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized.

In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Prolocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

In one embodiment, a binding molecule or antigen binding molecule for use in the methods of the invention comprises a synthetic constant region wherein one or more domains are partially or entirely deleted (“domain-deleted antibodies”). In certain embodiments compatible modified antibodies will comprise domain deleted constructs or variants wherein the entire C_{H}2 domain has been removed (ΔC_{H}2 constructs). For other embodiments a short connecting peptide may be substituted for the deleted domain to provide flexibility and freedom of movement for the variable region. Those skilled in the art will appreciate that such constructs are particularly preferred due to the regulatory properties of the C_{H}2 domain on the catabolic rate of the antibody.

In certain embodiments, modified antibodies for use in the methods disclosed herein are minibodies. Minibodies can be made using methods described in the art (see, e.g. U.S. Pat. No. 5,837,821 or WO 94/09817A1).

In another embodiment, modified antibodies for use in the methods disclosed herein are C_{H}2 domain deleted antibodies which are known in the art. Domain deleted constructs can be derived using a vector (e.g., from Biogen IDEC Incorporated) encoding an IgG1 human constant domain (see, e.g., WO 02/060955A2 and WO02/096948). This exemplary vector was engineered to delete the C_{H}2 domain and provide a synthetic vector expressing a domain deleted IgG₁ constant region.

In one embodiment, a OMgp antagonist antibody or fragment thereof for use in the treatment methods disclosed herein comprises an immunoglobulin heavy chain having deletion or substitution of a few or even a single amino acid as long as it permits association between the monomeric subunits. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct.

In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides the use of antibodies that comprise, consist essentially of, or consist of, variants (including derivatives) of antibody molecules (e.g., the V_{H} regions and/or V_{L} regions) described herein, which antibodies or fragments thereof immunospecifically bind to a OMgp polypeptide. Standard techniques known to those of skill in the art can be used to introduce mutations in the nucleotide sequence encoding a binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V_{H} region, V_{H}CDR₁, V_{H}CDR₂, V_{H}CDR₃, V_{L} region, V_{L}CDR₁, V_{L}CDR₂, or V_{L}CDR₃. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity.

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein can be determined using techniques described herein or by routinely modifying techniques known in the art.

### Fusion Proteins and Conjugated Polypeptides and Antibodies

OMgp polypeptides and antibodies for use in the treatment methods disclosed herein may further be recombinantly fused to a heterologous polypeptide at the N- or C-terminus or chemically conjugated (including covalent and non-covalent conjugations) to polypeptides or other compositions. For example, OMgp antagonist polypeptides or antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

OMgp antagonist polypeptides and antibodies for use in the treatment methods disclosed herein include derivatives that are modified, i.e., by the covalent attachment of any type of molecule such that covalent attachment does not prevent the OMgp antagonist polypeptide or antibody from inhibiting the biological function of OMgp. For example, but not by way of limitation, the OMgp antagonist polypeptides and antibodies of the present invention may be modified e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

OMgp antagonist polypeptides and antibodies for use in the treatment methods disclosed herein can be composed of amino acids joined to each other by peptide bonds or modified peptide bonds, i.e., peptide isosteres, and may contain amino acids other than the 20 gene-encoded amino acids. OMgp antagonist polypeptides and antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the OMgp antagonist polypeptide or antibody, including the peptide backbone, the amino acid side-chains and the amino or carboxyl termini, or on moieties such as carbohydrates. It will be appreciated that the same type of modification may be present in the same or varying degrees at several sites in a given OMgp antagonist polypeptide or antibody. Also, a given OMgp antagonist polypeptide or antibody may contain many types of modifications. OMgp antagonist polypeptides or antibodies may be branched, for example, as a result of ubiquitination, and they may be cyclic, with or without branching. Cyclic, branched, and branched cyclic OMgp antagonist polypeptides and antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising, consisting essentially of, or consisting of a OMgp antagonist polypeptide or antibody fusion that inhibits OMgp function. Preferably, the heterologous polypeptide to which the OMgp antagonist polypeptide or antibody is fused is useful for function or is useful to target the OMgp antagonist polypeptide or antibody. In certain embodiments of the invention a soluble OMgp antagonist polypeptide, e.g., an OMgp polypeptide comprising the LRR domains, CR domain, S/TR domain or the entire mature protein (corresponding to amino acids 25 to 425 of SEQ ID NO: 2), is fused to a heterologous polypeptide moiety to form a OMgp antagonist fusion polypeptide. OMgp antagonist fusion proteins and antibodies can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the OMgp antagonist polypeptide or antibody or to be cleavable, in vitro or in vivo. Heterologous moieties to accomplish these other objectives are known in the art.

As an alternative to expression of an OMgp antagonist fusion polypeptide or antibody, a chosen heterologous moiety can be preformed and chemically conjugated to the OMgp antagonist polypeptide or antibody. In most cases, a chosen heterologous moiety will function similarly, whether fused or conjugated to the OMgp antagonist polypeptide or antibody. Therefore, in the following discussion of heterologous amino acid sequences, unless otherwise noted, it is to be understood that the heterologous sequence can be joined to the OMgp antagonist polypeptide or antibody in the form of a fusion protein or as a chemical conjugate.

Pharmacologically active polypeptides such as OMgp antagonist polypeptides or antibodies often exhibit rapid in vivo clearance, necessitating large doses to achieve therapeutically effective concentrations in the body. In addition, polypeptides smaller than about 60 kDa potentially undergo glomerular filtration, which sometimes leads to nephrotoxicity. Fusion or conjugation of relatively small polypeptides such as OMgp antagonist polypeptides or antibodies can be employed to reduce or avoid the risk of such nephrotoxicity. Various heterologous amino acid sequences, i.e., polypeptide moieties or “carriers,” for increasing the in vivo stability, i.e., serum half-life, of therapeutic polypeptides are known.

Due to its long half-life, wide in vivo distribution, and lack of enzymatic or immunological function, essentially full-length human serum albumin (HSA), or an HSA fragment, is commonly used as a heterologous moiety. Through application of methods and materials such as those taught in Yeh et al., Proc. Natl. Acad. Sci. USA 89:1904-08 (1992) and Syed et al., Blood 89:3243-52 (1997), HSA can be used to form an OMgp antagonist fusion polypeptide or antibody or polypeptide/antibody conjugate that displays pharmacological activity by virtue of the OMgp moiety while displaying significantly increased in vivo stability, e.g., 10-fold to 100-fold higher. The C-terminus of the HSA can be fused to the N-terminus of the soluble OMgp moiety. Since HSA is a naturally secreted protein, the HSA signal sequence can be exploited to obtain secretion of the soluble OMgp fusion protein into the cell culture medium when the fusion protein is produced in a eukaryotic, e.g., mammalian, expression system.

The signal sequence is a polynucleotide that encodes an amino acid sequence that initiates transport of a protein across the membrane of the endoplasmic reticulum. Signal sequences useful for constructing an immunofusin include antibody light chain signal sequences, e.g., antibody 14.18 (Gillies et al., J. Immunol. Meth. 125:191-202 (1989)), antibody heavy chain signal sequences, e.g., the MOPC141 antibody heavy chain signal sequence (Sakano et al., Nature 286:5774 (1980)). Alternatively, other signal sequences can be used. See, e.g., Watson, Nucl. Acids Res. 12:5145 (1984). The signal peptide is usually cleaved in the lumen of the endoplasmic reticulum by signal peptidases. This results in the secretion of an immunofusin protein containing the Fc region and the soluble OMgp moiety.

In some embodiments, the DNA sequence may encode a proteolytic cleavage site between the secretion cassette and the soluble OMgp moiety. Such a cleavage site may provide, e.g., for the proteolytic cleavage of the encoded fusion protein, thus separating the Fc domain from the target protein. Useful proteolytic cleavage sites include amino acid sequences recognized by proteolytic enzymes such as trypsin, plasmin, thrombin, factor Xa, or enterokinase K.

The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., Biochim. Biophys. Acta 1088:712 (1991); and Lo et al., Protein Engineering 11:495-500 (1998). An appropriate host cell can be transformed or transfected with a DNA that encodes a soluble OMgp polypeptide and used for the expression and secretion of the soluble OMgp polypeptide. Host cells that are typically used include immortal hybridoma cells, myeloma cells, 293 cells, Chinese hamster ovary (CHO) cells, Hela cells, and COS cells.

In one embodiment, a soluble OMgp polypeptide is fused to a hinge and Fc region, i.e., the C-terminal portion of an Ig heavy chain constant region. Potential advantages of an OMgp-Fc fusion include solubility, in vivo stability, and multivalency, e.g., dimerization. The Fc region used can be an IgA, IgD, or IgG Fc region (hinge-C_{H}2-C_{H}3). Alternatively, it can be an IgE or IgM Fc region (hinge-C_{H}2-C_{H}3-C_{H}4). An IgG Fc region is generally used, e.g., an IgG₁ Fc region or IgG₄ Fc region. In one embodiment, a sequence beginning in the hinge region just upstream of the papain cleavage site which defines IgG Fc chemically (i.e. residue 216, taking the first residue of heavy chain constant region to be 114 according to the Kabat system), or analogous sites of other immunoglobulins is used in the fusion. The precise site at which the fusion is made is not critical; particular sites are well known and may be selected in order to optimize the biological activity, secretion, or binding characteristics of the molecule. Materials and methods for constructing and expressing DNA encoding Fc fusions are known in the art and can be applied to obtain soluble OMgp fusions without undue experimentation. Some embodiments of the invention employ an OMgp fusion protein such as those described in Capon et al., U.S. Pat. Nos. 5,428,130 and 5,565,335.

Fully intact, wild-type Fc regions display effector functions that normally are unnecessary and undesired in an Fc fusion protein used in the methods of the present invention. Therefore, certain binding sites typically are deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the C_{H}2 domain of the Fc region of IgE, such that this site does not interfere with the efficient secretion of the immunofusin. Transmembrane domain sequences, such as those present in IgM, also are generally deleted.

The IgG1 Fc region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the C_{H}2 region, and the C_{H}3 region. In some embodiments, the Fc region of immunoglobulin gamma-1 is a C_{H}2-deleted-Fc, which includes part of the hinge region and the C_{H}3 region, but not the C_{H}2 region. A C_{H}2-deleted-Fc has been described by Gillies et al., Hum. Antibod. Hybridomas 1:47 (1990). In some embodiments, the Fc region of one of IgA, IgD, IgE, or IgM, is used.

OMgp-Fc fusion proteins can be constructed in several different configurations. In one configuration the C-terminus of the soluble OMgp moiety is fused directly to the N-terminus of the Fc hinge moiety. In a slightly different configuration, a short polypeptide, e.g., 2-10 amino acids, is incorporated into the fusion between the N-terminus of the soluble OMgp moiety and the C-terminus of the Fc moiety. Such a linker provides conformational flexibility, which may improve biological activity in some circumstances. If a sufficient portion of the hinge region is retained in the Fc moiety, the OMgp-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Any of a number of cross-linkers that contain a corresponding amino-reactive group and thiol-reactive group can be used to link OMgp antagonist polypeptides to serum albumin. Examples of suitable linkers include amine reactive cross-linkers that insert a thiol-reactive maleimide, e.g., SMCC, AMAS, BMPS, MBS, EMCS, SMPB, SMPH, KMUS, and GMBS. Other suitable linkers insert a thiol-reactive haloacetate group, e.g., SBAP, SIA, SIAB. Linkers that provide a protected or non-protected thiol for reaction with sulfhydryl groups to product a reducible linkage include SPDP, SMPT, SATA, and SATP. Such reagents are commercially available (e.g., Pierce Chemicals).

Conjugation does not have to involve the N-terminus of a soluble OMgp polypeptide or the thiol moiety on serum albumin. For example, soluble OMgp-albumin fusions can be obtained using genetic engineering techniques, wherein the soluble OMgp moiety is fused to the serum albumin gene at its N-terminus, C-terminus, or both.

Soluble OMgp polypeptides can be fused to heterologous peptides to facilitate purification or identification of the soluble OMgp moiety. For example, a histidine tag can be fused to a soluble OMgp polypeptide to facilitate purification using commercially available chromatography media.

In some embodiments of the invention, a soluble OMgp fusion construct is used to enhance the production of a soluble OMgp moiety in bacteria. In such constructs a bacterial protein normally expressed and/or secreted at a high level is employed as the N-terminal fusion partner of a soluble OMgp polypeptide. See, e.g., Smith et al., Gene 67:31 (1988); Hopp et al., Biotechnology 6:1204 (1988); La Vallie et al., Biotechnology 11:187 (1993).

By fusing a soluble OMgp moiety at the amino and carboxy termini of a suitable fusion partner, bivalent or tetravalent forms of a soluble OMgp polypeptide can be obtained. For example, a soluble OMgp moiety can be fused to the amino and carboxy termini of an Ig moiety to produce a bivalent monomeric polypeptide containing two soluble OMgp moieties. Upon dimerization of two of these monomers, by virtue of the Ig moiety, a tetravalent form of a soluble OMgp protein is obtained. Such multivalent forms can be used to achieve increased binding affinity for the target. Multivalent forms of soluble OMgp also can be obtained by placing soluble OMgp moieties in tandem to form concatamers, which can be employed alone or fused to a fusion partner such as Ig or HSA.

Conjugated Polymers (Other than Polypeptides)

Some embodiments of the invention involve a soluble OMgp polypeptide or OMgp antibody wherein one or more polymers are conjugated (covalently linked) to the OMgp polypeptide or antibody. Examples of polymers suitable for such conjugation include polypeptides (discussed above), sugar polymers and polyalkylene glycol chains. Typically, but not necessarily, a polymer is conjugated to the soluble OMgp polypeptide or OMgp antibody for the purpose of improving one or more of the following: solubility, stability, or bioavailability.

The class of polymer generally used for conjugation to a OMgp antagonist polypeptide or antibody is a polyalkylene glycol. Polyethylene glycol (PEG) is most frequently used. PEG moieties, e.g., 1, 2, 3, 4 or 5 PEG polymers, can be conjugated to each OMgp antagonist polypeptide or antibody to increase serum half life, as compared to the OMgp antagonist polypeptide or antibody alone. PEG moieties are non-antigenic and essentially biologically inert. PEG moieties used in the practice of the invention may be branched or unbranched.

The number of PEG moieties attached to the OMgp antagonist polypeptide or antibody and the molecular weight of the individual PEG chains can vary. In general, the higher the molecular weight of the polymer, the fewer polymer chains attached to the polypeptide. Usually, the total polymer mass attached to the OMgp antagonist polypeptide or antibody is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the OMgp antagonist polypeptide or antibody through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the OMgp antagonist polypeptide or antibody. Free carboxylic groups, suitably activated carbonyl groups, hydroxyl, guanidyl, imidazole, oxidized carbohydrate moieties and mercapto groups of the OMgp antagonist polypeptide or antibody (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the OMgp antagonist polypeptide or antibody. Preferably, at least 50% of the biological activity (as demonstrated, e.g., in any of the assays described herein or known in the art) of the OMgp antagonist polypeptide or antibody is retained, and most preferably nearly 100% is retained.

The polymer can be conjugated to the OMgp antagonist polypeptide or antibody using conventional chemistry. For example, a polyalkylene glycol moiety can be coupled to a lysine epsilon amino group of the QMgp antagonist polypeptide or antibody. Linkage to the lysine side chain can be performed with an N-hydroxylsuccinimide (NHS) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors 3:4-10 (1992), and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5:133-140, 1994. Reaction parameters are generally selected to avoid temperature, solvent, and pH conditions that would damage or inactivate the soluble OMgp polypeptide.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with OMgp antagonist polypeptide or antibody in the presence of a reducing agent. In addition, one can manipulate the reaction conditions to favor PEGylation substantially only at the N-terminal amino group of OMgp antagonist polypeptide or antibody, i.e. a mono-PEGylated protein. In either case of mono-PEGylation or poly-PEGylation, the PEG groups are typically attached to the protein via a —CH2-NH— group. With particular reference to the —CH2-group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C1-C10 alkoxy or aryloxy derivatives thereof (see, e.g., Harris et al., U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing a PEGylated soluble OMgp polypeptide or antibody generally includes the steps of (a) reacting a OMgp antagonist polypeptide or antibody with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/soluble OMgp polypeptide or OMgp antibody generally includes the steps of: (a) reacting a soluble OMgp protein or polypeptide with a reactive PEG molecule under reductive alkylation conditions, at a pH suitable to pen-nit selective modification of the N-terminal amino group of the polypeptide or antibody; and (b) obtaining the reaction product(s).

For a substantially homogeneous population of mono-polymer/soluble OMgp polypeptide or OMgp antibody, the reductive alkylation reaction conditions are those that permit the selective attachment of the water-soluble polymer moiety to the N-terminus of the polypeptide or antibody. Such reaction conditions generally provide for pKa differences between the lysine side chain amino groups and the N-terminal amino group. For purposes of the present invention, the pH is generally in the range of 3-9, typically 3-6.

Soluble OMgp polypeptides or antibodies can include a tag, e.g., a moiety that can be subsequently released by proteolysis. Thus, the lysine moiety can be selectively modified by first reacting a His-tag modified with a low-molecular-weight linker such as Traut's reagent (Pierce) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SLAB), or a vinylsulfone group and react the resulting product with a PEG that contains a free SH.

In some embodiments, the polyalkylene glycol moiety is coupled to a cysteine group of the OMgp antagonist polypeptide or antibody. Coupling can be effected using, e.g., a maleimide group, a vinylsulfone group, a haloacetate group, or a thiol group.

Optionally, the soluble OMgp polypeptide or antibody is conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

### OMgp Polynucleotide Antagonists

Specific embodiments comprise a method of treating a demyelination or dysmyelination disorder, comprising administering an effective amount of an OMgp polynucleotide antagonist which comprises a nucleic acid molecule which specifically binds to a polynucleotide which encodes OMgp. The OMgp polynucleotide antagonist prevents expression of OMgp (knockdown). OMgp polynucleotide antagonists include, but are not limited to antisense molecules, ribozymes, siRNA, shRNA and RNAi. Typically, such binding molecules are separately administered to the animal (see, for example, O'Connor, J. Neurochem. 56:560 (1991), but such binding molecules may also be expressed in vivo from polynucleotides taken up by a host cell and expressed in vivo. See also Oligodeoxynucleotides as Antisense Inhibitors of Gene Expression, CRC Press, Boca Raton, Fla. (1988).

RNAi refers to the expression of an RNA which interferes with the expression of the targeted mRNA. Specifically, the RNAi silences a targeted gene via interacting with the specific mRNA (e.g. OMgp) through a siRNA (short interfering RNA). The ds RNA complex is then targeted for degradation by the cell. Additional RNAi molecules include Short hairpin RNA (shRNA); also short interfering hairpin. The shRNA molecule contains sense and antisense sequences from a target gene connected by a loop. The shRNA is transported from the nucleus into the cytoplasm, it is degraded along with the mRNA. Pol III or U6 promoters can be used to express RNAs for RNAi.

RNAi is mediated by double stranded RNA (dsRNA) molecules that have sequence-specific homology to their “target” mRNAs (Caplen et al., Proc Natl Acad Sci USA 98:9742-9747, 2001). Biochemical studies in Drosophila cell-free lysates indicates that the mediators of RNA-dependent gene silencing are 21-25 nucleotide “small interfering” RNA duplexes (siRNAs). Accordingly, siRNA molecules are advantageously used in the methods of the present invention. The siRNAs are derived from the processing of dsRNA by an RNase known as DICER (Bernstein et al., Nature 409:363-366, 2001). It appears that siRNA duplex products are recruited into a multi-protein siRNA complex termed RISC(RNA Induced Silencing Complex). Without wishing to be bound by any particular theory, it is believed that a RISC is guided to a target mRNA, where the siRNA duplex interacts sequence-specifically to mediate cleavage in a catalytic fashion (Bernstein et al., Nature 409:363-366, 2001; Boutla et al., Curr Biol 11:1776-1780, 2001).

RNAi has been used to analyze gene function and to identify essential genes in mammalian cells (Elbashir et al., Methods 26:199-213, 2002; Harborth et al., J Cell Sci 114:4557-4565, 2001), including by way of non-limiting example neurons (Krichevsky et al., Proc Natl Acad Sci USA 99:11926-11929, 2002). RNAi is also being evaluated for therapeutic modalities, such as inhibiting or blocking the infection, replication and/or growth of viruses, including without limitation poliovirus (Gitlin et al., Nature 418:379-380, 2002) and HIV (Capodici et al., J Immunol 169:5196-5201, 2002), and reducing expression of oncogenes. RNAi has been used to modulate gene expression in mammalian (mouse) and amphibian (Xenopus) embryos (respectively, Calegari et al., Proc Natl Acad Sci USA 99:14236-14240, 2002; and Zhou, et al., Nucleic Acids Res 30:1664-1669, 2002), and in postnatal mice (Lewis et al., Nat Genet 32:107-108, 2002), and to reduce trangsene expression in adult transgenic mice (McCaffrey et al., Nature 418:38-39, 2002). Methods have been described for determining the efficacy and specificity of siRNAs in cell culture and in vivo (see, e.g., Bertrand et al., Biochem Biophys Res Commun 296:1000-1004, 2002; Lassus et al., Sci STKE 2002 (147):PL13, 2002; and Leirdal et al., Biochem Biophys Res Commun 295:744-748, 2002).

Molecules that mediate RNAi, including without limitation siRNA, can be produced in vitro by chemical synthesis (Hohjoh, FEBS Lett 521:195-199, 2002), hydrolysis of dsRNA (Yang et al., Proc Natl Acad Sci USA 99:9942-9947, 2002), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947, 2002).

siRNA molecules may also be formed by annealing two oligonucleotides to each other, typically have the following general structure, which includes both double-stranded and single-stranded portions:

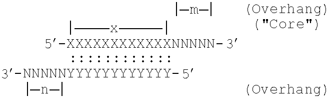

Wherein N, X and Y are nucleotides; X hydrogen bonds to Y; “:” signifies a hydrogen bond between two bases; x is a natural integer having a value between 1 and about 100; and m and n are whole integers having, independently, values between 0 and about 100. In some embodiments, N, X and Y are independently A, G, C and T or U. Non-naturally occurring bases and nucleotides can be present, particularly in the case of synthetic siRNA (i.e., the product of annealing two oligonucleotides). The double-stranded central section is called the “core” and has base pairs (bp) as units of measurement; the single-stranded portions are overhangs, having nucleotides (nt) as units of measurement. The overhangs shown are 3′ overhangs, but molecules with 5′ overhangs are also within the scope of the invention. Also within the scope of the invention are siRNA molecules with no overhangs (i.e., m=0 and n=0), and those having an overhang on one side of the core but not the other (e.g., m=0 and n>1, or vice-versa).

Initially, RNAi technology did not appear to be readily applicable to mammalian systems. This is because, in mammals, dsRNA activates dsRNA-activated protein kinase (PKR) resulting in an apoptotic cascade and cell death (Der et al., Proc. Natl. Acad. Sci. USA 94:3279-3283, 1997). In addition, it has long been known that dsRNA activates the interferon cascade in mammalian cells, which can also lead to altered cell physiology (Colby et al., Annu. Rev. Microbiol. 25:333, 1971; Kleinschmidt et al., Annu. Rev. Biochem. 41:517, 1972; Lampson et al., Proc. Natl. Acad. Sci. USA 58L782, 1967; Lomniczi et al., J. Gen. Virol. 8:55, 1970; and Younger et al., J. Bacteriol. 92:862, 1966). However, dsRNA-mediated activation of the PKR and interferon cascades requires dsRNA longer than about 30 base pairs. In contrast, dsRNA less than 30 base pairs in length has been demonstrated to cause RNAi in mammalian cells (Caplen et al., Proc. Natl. Acad. Sci. USA 98:9742-9747, 2001). Thus, it is expected that undesirable, non-specific effects associated with longer dsRNA molecules can be avoided by preparing short RNA that is substantially free from longer dsRNAs.

References regarding siRNA: Bernstein et al., Nature 409:363-366, 2001; Boutla et al., Curr Biol 11:1776-1780, 2001; Cullen, Nat Immunol. 3:597-599, 2002; Caplen et al., Proc Natl Acad Sci USA 98:9742-9747, 2001; Hamilton et al., Science 286:950-952, 1999; Nagase et al., DNA Res. 6:63-70, 1999; Napoli et al., Plant Cell 2:279-289, 1990; Nicholson et al., Mamm. Genome 13:67-73, 2002; Parrish et al., Mol Cell 6:1077-1087, 2000; Romano et al., Mol Microbiol 6:3343-3353, 1992; Tabara et al., Cell 99:123-132, 1999; and Tuschl, Chembiochem. 2:239-245, 2001.

Paddison et al. (Genes & Dev. 16:948-958, 2002) have used small RNA molecules folded into hairpins as a means to effect RNAi. Accordingly, such short hairpin RNA (shRNA) molecules are also advantageously used in the methods of the invention. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the dsRNA products of the DICER RNase and, in any event, have the same capacity for inhibiting expression of a specific gene.

In some embodiments of the invention, the shRNA is expressed from a lentiviral vector.

Antisense technology can be used to control gene expression through antisense DNA or RNA, or through triple-helix formation. Antisense techniques are discussed for example, in Okano, J. Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes OMgp may be used to design an antisense RNA oligonucleotide of from about 10 to 40 base pairs in length. A DNA oligonucleotide is designed to be complementary to a region of the gene involved in transcription thereby preventing transcription and the production of the target protein. The antisense RNA oligonucleotide hybridizes to the mRNA in vivo and blocks translation of the mRNA molecule into the target polypeptide.

In one embodiment, antisense nucleic acids specific for the OMgp gene are produced intracellularly by transcription from an exogenous sequence. For example, a vector or a portion thereof, is transcribed, producing an antisense nucleic acid (RNA). Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the antisense molecule, can be by any promoter known in the art to act in vertebrate, preferably human cells, such as those described elsewhere herein.

Absolute complementarity of an antisense molecule, although preferred, is not required. A sequence complementary to at least a portion of an RNA encoding OMgp, means a sequence having sufficient complementarity to be able to hybridize with the RNA, forming a stable duplex; or triplex formation may be assayed. The ability to hybridize will depend on both the degree of complementarity and the length of the antisense nucleic acid. Generally, the larger the hybridizing nucleic acid, the more base mismatches it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of a messenger RNA, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature 372:333-335 (1994). Thus, oligonucleotides complementary to either the 5′- or 3′-non-translated, non-coding regions could be used in an antisense approach to inhibit translation of OMgp. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Polynucleotides for use the therapeutic methods disclosed herein can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. 84:648-652 (1987)); PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., BioTechniques 6:958-976 (1988)) or intercalating agents. (See, e.g., Zon, Pharm. Res. 5:539-549 (1988)). To this end, the oligonucleotide may be conjugated to another molecule, e.g., a peptide, hybridization triggered cross-linking agent, transport agent, hybridization-triggered cleavage agent, etc.

An antisense oligonucleotide for use in the therapeutic methods disclosed herein may comprise at least one modified base moiety which is selected from the group including, but not limited to, 5fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3(3-amino-3-N2-carboxypropyl) uracil, (acp3)w, and 2,6-diaminopurine.

An antisense oligonucleotide for use in the therapeutic methods disclosed herein may also comprise at least one modified sugar moiety selected from the group including, but not limited to, arabinose, 2-fluoroarabinose, xylulose, and hexose.

In yet another embodiment, an antisense oligonucleotide for use in the therapeutic methods disclosed herein comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, an antisense oligonucleotide for use in the therapeutic methods disclosed herein is an α-anomeric oligonucleotide. An α-anomeric oligonucleotide forms specific double-stranded hybrids with complementary RNA in which, contrary to the usual situation, the strands run parallel to each other (Gautier et al., Nucl. Acids Res. 15:6625-6641 (1987)). The oligonucleotide is a 2′-0-methylribonucleotide (Inoue et al., Nucl. Acids Res. 15:6131-6148 (1987)), or a chimeric RNA-DNA analogue (Inoue et al., FEBS Lett. 215:327-330 (1987)).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), methylphosphonate oligonucleotides can be prepared by use of controlled pore glass polymer supports (Sarin et al., Proc. Natl. Acad. Sci. U.S.A. 85:7448-7451 (1988)), etc.

Polynucleotide compositions for use in the therapeutic methods disclosed herein further include catalytic RNA, or a ribozyme (See, e.g., PCT International Publication WO 90/11364, published Oct. 4, 1990; Sarver et al., Science 247:1222-1225 (1990). The use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). Preferably, the ribozyme is engineered so that the cleavage recognition site is located near the 5′ end of the target mRNA; i.e., to increase efficiency and minimize the intracellular accumulation of non-functional mRNA transcripts.

As in the antisense approach, ribozymes for use in the diagnostic and therapeutic methods disclosed herein can be composed of modified oligonucleotides (e.g. for improved stability, targeting, etc.) and may be delivered to cells which express OMgp in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous OMgp messages and inhibit translation. Since ribozymes unlike antisense molecules, are catalytic, a lower intracellular concentration is required for efficiency.

### Vectors

Vectors comprising nucleic acids encoding OMgp antagonists may also be used to produce antagonists for use in the methods of the invention. The choice of vector and expression control sequences to which such nucleic acids are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status, or a change in temperature, in the host cell medium. Example of inducible promoters include, but are not limited to the metallothionein promoter, heat shock promoters, the albumin promoter, the ApoAI promoter, human globin promoters, the β-actin promoter, and human growth hormone promoters.

The vector can include a prokaryotic replicon, i.e., a DNA sequence having the ability to direct autonomous replication and maintenance of the recombinant DNA molecule extra-chromosomally in a bacterial host cell. Such replicons are well known in the art. In addition, vectors that include a prokaryotic replicon may also include a gene whose expression confers a detectable marker such as a drug resistance. Examples of bacterial drug-resistance genes are those that confer resistance to ampicillin or tetracycline.

Vectors that include a prokaryotic replicon can also include a prokaryotic or bacteriophage promoter for directing expression of the coding gene sequences in a bacterial host cell. Promoter sequences compatible with bacterial hosts are typically provided in plasmid vectors containing convenient restriction sites for insertion of a DNA segment to be expressed. Examples of such plasmid vectors are pUC8, pUC9, pBR322 and pBR329 (BioRad), pPL and pKK223 (Pharmacia). Any suitable prokaryotic host can be used to express a recombinant DNA molecule encoding a protein used in the methods of the invention.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. The neomycin phosphotransferase (neo) gene is an example of a selectable marker gene (Southern et al., J. Mol. Anal. Genet. 1:327-341 (1982)). Additional elements may also be needed for optimal synthesis of mRNA. These elements may include signal sequences, splice signals, as well as transcriptional promoters, enhancers, and termination signals.

In one embodiment, a proprietary expression vector of Biogen IDEC, Inc., referred to as NEOSPLA (U.S. Pat. No. 6,159,730) may be used. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression upon transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). Additional eukaryotic cell expression vectors are known in the art and are commercially available. Typically, such vectors contain convenient restriction sites for insertion of the desired DNA segment. Exemplary vectors include pSVL and pKSV-10 (Pharmacia), pBPV-1, pml2d (International Biotechnologies), pTDT1 (ATCC 31255), retroviral expression vector pMIG and pLL3.7, adenovirus shuttle vector pDC315, and AAV vectors. Other exemplary vector systems are disclosed e.g., in U.S. Pat. No. 6,413,777.

In general, screening large numbers of transformed cells for those which express suitably high levels of the antagonist is routine experimentation which can be carried out, for example, by robotic systems.

Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdmlP)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The recombinant expression vectors may carry sequences that regulate replication of the vector in host cells (e.g., origins of replication) and selectable marker genes. The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Vectors encoding OMgp antagonists can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., Proc. Natl. Acad. Sci. USA 69:2110-14 (1972)). For transformation of vertebrate cells, electroporation, cationic lipid or salt treatment methods can be employed. See, e.g., Graham et al., Virology 52:456-467 (1973); Wigler et al., Proc. Natl. Acad. Sci. USA 76:1373-76 (1979).

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to NSO, SP2 cells, baby hamster kidney (BHK) cells, monkey kidney cells (COS), human hepatocellular carcinoma cells (e.g., Hep G2), A549 cells DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

### Host Cells

Host cells for expression of an OMgp antagonist for use in a method of the invention may be prokaryotic or eukaryotic. Exemplary eukaryotic host cells include, but are not limited to, yeast and mammalian cells, e.g., Chinese hamster ovary (CHO) cells (ATCC Accession No. CCL61), NIH Swiss mouse embryo cells NIH-3T3 (ATCC Accession No. CRL1658), and baby hamster kidney cells (BHK). Other useful eukaryotic host cells include insect cells and plant cells. Exemplary prokaryotic host cells are E. coli and Streptomyces.

### Gene Therapy

An OMgp antagonist can be produced in vivo in a mammal, e.g., a human patient, using a gene-therapy approach to treatment of a nervous-system disease, disorder or injury in which promoting survival, proliferation and differentiation of neurons and/or oligodendrocytes or promoting myelination of neurons would be therapeutically beneficial. This involves administration of a suitable OMgp antagonist-encoding nucleic acid operably linked to suitable expression control sequences. Generally, these sequences are incorporated into a viral vector. Suitable viral vectors for such gene therapy include an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, adeno-associated viral vector and a herpes simplex viral vector. The viral vector can be a replication-defective viral vector. Adenoviral vectors that have a deletion in its E1 gene or E3 gene are typically used. When an adenoviral vector is used, the vector usually does not have a selectable marker gene.

### Pharmaceutical Compositions

The OMgp antagonists used in the methods of the invention may be formulated into pharmaceutical compositions for administration to mammals, including humans. The pharmaceutical compositions used in the methods of this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. As described previously, OMgp anatgonists of the invention act in the nervous system to promote survival, proliferation and differentiation of neurons and/or oligodendrocytes, as well as myelination of neurons. Accordingly, in the methods of the invention, the OMgp antagonists are administered in such a way that they cross the blood-brain barrier. This crossing can result from the physico-chemical properties inherent in the OMgp antagonist molecule itself, from other components in a pharmaceutical formulation, or from the use of a mechanical device such as a needle, cannula or surgical instruments to breach the blood-brain barrier. Where the OMgp antagonist is a molecule that does not inherently cross the blood-brain barrier, e.g., a fusion to a moiety that facilitates the crossing, suitable routes of administration are, e.g., intrathecal or intracranial, e.g., directly into a chronic lesion of MS. Where the OMgp antagonist is a molecule that inherently crosses the blood-brain barrier, the route of administration may be by one or more of the various routes described below. In some methods, antibodies are administered as a sustained release composition or device, such as a Medipad™ device. Delivery across the blood brain barrier can be enhanced by a carrying molecule, such as anti-Fc receptor, transferrin, anti-insulin receptor or a toxin conjugate or penetration enhancer.

Sterile injectable forms of the compositions used in the methods of this invention may be aqueous or oleaginous suspension. These suspensions may be formulated according to techniques known in the art using suitable dispersing or wetting agents and suspending agents. The sterile, injectable preparation may also be a sterile, injectable solution or suspension in a non-toxic parenterally acceptable diluent or solvent, for example as a suspension in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in the methods of this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

The amount of an OMgp antagonist that may be combined with the carrier materials to produce a single dosage form will vary depending upon the host treated, the type of antagonist used and the particular mode of administration. The composition may be administered as a single dose, multiple doses or over an established period of time in an infusion. Dosage regimens also may be adjusted to provide the optimum desired response (e.g., a therapeutic or prophylactic response).

The methods of the invention use a “therapeutically effective amount” or a “prophylactically effective amount” of an OMgp antagonist. Such a therapeutically or prophylactically effective amount may vary according to factors such as the disease state, age, sex, and weight of the individual. A therapeutically or prophylactically effective amount is also one in which any toxic or detrimental effects are outweighed by the therapeutically beneficial effects.

A specific dosage and treatment regimen for any particular patient will depend upon a variety of factors, including the particular OMgp antagonist used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

In the methods of the invention the OMgp antagonists are generally administered directly to the nervous system, intracerebroventricularly, or intrathecally, e.g. into a chronic lesion of MS. Compositions for administration according to the methods of the invention can be formulated so that a dosage of 0.001-10 mg/kg body weight per day of the OMgp antagonist polypeptide is administered. In some embodiments of the invention, the dosage is 0.01-1.0 mg/kg body weight per day. In some embodiments, the dosage is 0.001-0.5 mg/kg body weight per day.

For treatment with an OMgp antagonist antibody, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. In some methods, dosage is adjusted to achieve a plasma polypeptide concentration of 1-1000 μg/ml and in some methods 25-300 μg/ml. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

In certain embodiments, a subject can be treated with a nucleic acid molecule encoding a OMgp antagonist polynucleotide. Doses for nucleic acids range from about 10 ng to 1 g, 100 ng to 100 mg, 1 μg to 10 mg, or 30-300 μg DNA per patient. Doses for infectious viral vectors vary from 10-100, or more, virions per dose.

Supplementary active compounds also can be incorporated into the compositions used in the methods of the invention. For example, a soluble OMgp polypeptide or a fusion protein may be coformulated with and/or coadministered with one or more additional therapeutic agents.

The invention encompasses any suitable delivery method for an OMgp antagonist to a selected target tissue, including bolus injection of an aqueous solution or implantation of a controlled-release system. Use of a controlled-release implant reduces the need for repeat injections.

OMgp antagonists of the invention can be administered on multiple occasions. Intervals between single dosages can be daily, weekly, monthly or yearly. Intervals can also be irregular as indicated by measuring blood levels of target polypeptide or target molecule in the patient. Alternatively, OMgp anatgonists of the invention can be administered as a sustained release formulation, in which case less frequent administration is required. Dosage and frequency vary depending on the half-life of the OMgp anatgonist in the patient. The half-life of a soluble OMgp polypeptide or OMgp antibody, fragment, variant or derivative thereof can also be prolonged via fusion to a stable polypeptide or moiety, e.g., albumin or PEG. In general, humanized antibodies show the longest half-life, followed by chimeric antibodies and nonhuman antibodies. In one embodiment, the OMgp anatgonists of the invention can be administered in unconjugated form. In another embodiment, the OMgp antagonists of the invention can be administered multiple times in conjugated form. In still another embodiment, the OMgp antagonists of the invention can be administered in unconjugated form, then in conjugated form, or vice versa.

The OMgp antagonists used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., “High Activity Iodine-125 Interstitial Implant For Gliomas,” Int. J. Radiation Oncology Biol. Phys. 24(4):583-591 (1992); Gaspar et al., “Permanent 125I Implants for Recurrent Malignant Gliomas,” Int. J. Radiation Oncology Biol. Phys. 43(5):977-982 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

The compositions may also comprise an OMgp antagonist dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988).

In some embodiments of the invention, an OMgp antagonist is administered to a patient by direct infusion into an appropriate region of the brain. See, e.g., Gill et al., “Direct brain infusion of glial cell line-derived neurotrophic factor in Parkinson disease,” Nature Med. 9: 589-95 (2003). Alternative techniques are available and may be applied to administer an OMgp antagonist according to the invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

The methods of treatment of demyelination or dysmyelination disorders as described herein are typically tested in vitro, and then in vivo in an acceptable animal model, for the desired therapeutic or prophylactic activity, prior to use in humans. Suitable animal models, including transgenic animals, are will known to those of ordinary skill in the art. For example, in vitro assays to demonstrate the proliferation, differentiation and survival effect of the OMgp antagonists are described herein. The effect of the OMgp antagonists on myelination of axons can be tested in vitro as described in the Examples. Finally, in vivo tests can be performed by creating transgenic mice which express the OMgp antagonist or by administering the OMgp antagonist to mice or rats in models as described herein.

### EXAMPLES

### Example 1

### OMgp is Specifically Expressed in the Central Nervous System

Various rat and human tissues were examined for the expression of OMgp. A human Multiple Tissue Northern (BD MTN™) Blot (Clontech, Mountain View, Calif.) and a rat multiple tissue Northern blot (Cat. # RB2030, Origene, Rockville, Md.) were purchased which contained mRNA from various tissue types. Messenger RNA from brain, colon, heart, kidney, liver, lung, muscle, placenta, small intestine, spleen, stomach and testis were examined for OMgp mRNA transcription. The OMgp probe used with the previously mentioned blots was produced by PCR with the following primers:

| | |
|---|---|
| 5′ PCR Primer- | |
| 5′-GCCCACAGCCAGAAACAGTT-3′ | (SEQ ID NO:15) |
| 3′ PCR Primer- | |
| 5′-TCCAGGGTCCTCAGATTGGTAT-3′. | (SEQ ID NO:11) |

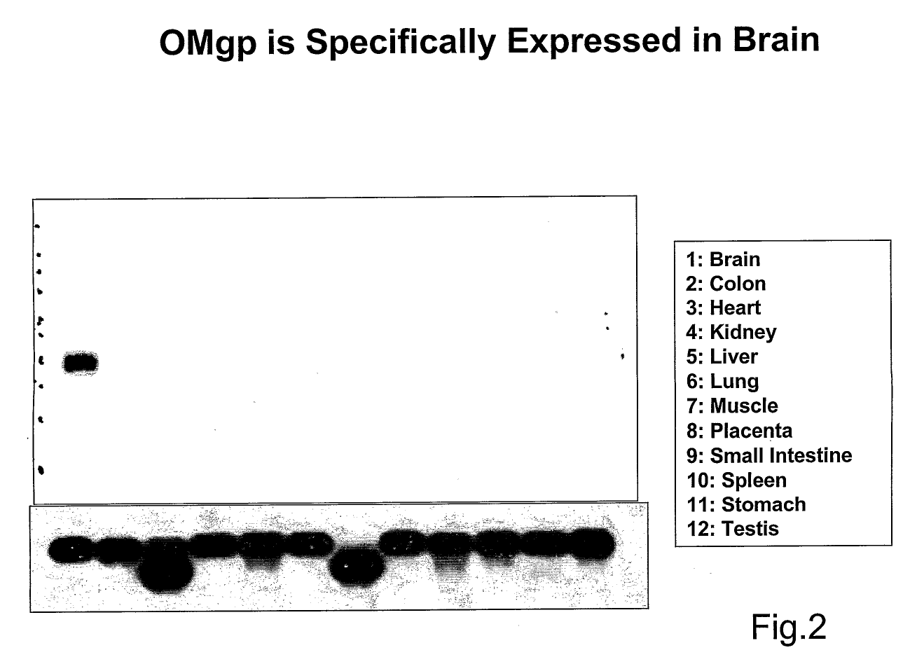

Northern blots were hybridized overnight at 68° C. with ³²P labeled OMgp probe (product of the PCR reaction described above). The blots were then washed 3 times with 2×SSC, 0.5% SDS and then three times with 0.5×SSC, 0.1% SDS. The blots were exposed to X-ray film and the mRNA levels were visualized by autoradiography. Of the 12 tissue types tested, only the brain tissue expressed OMgp. Results of the human Northern blot are shown in FIG. 2.

### OMgp is Developmentally Regulated in the Rat Brain and Spinal Cord

RT-PCR was used to examine the expression of OMgp at different time points during rat development in the brain and spinal cord. RT-PCR was performed as described in Sha et al., Nat. Neurosci. 8:745-51 (2005), which is hereby incorporated by reference. Primers used in the RT-PCR were the same as the primers used to create the OMgp probe used in the Northern experiment described above (i.e. SEQ ID NOs: 10 and 11).

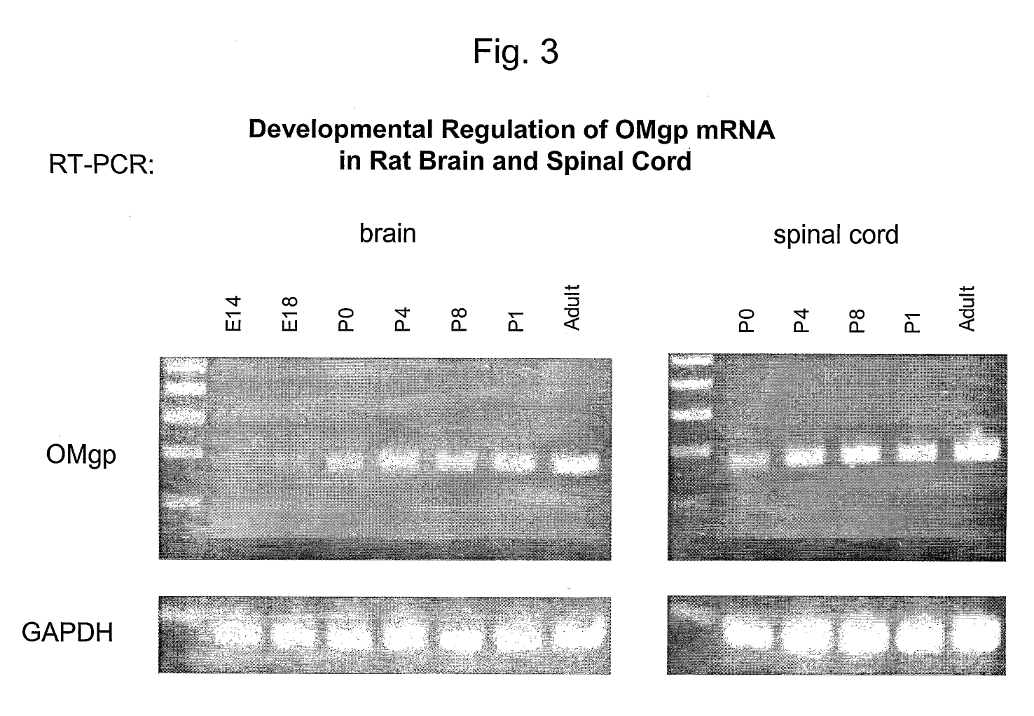

In both the rat brain and spinal cord, OMgp expression increases as the rat develops. By the time the rat reaches the Adult stage (1 month old), OMgp expression is at its highest level. See FIG. 3. Indeed, at embryonic day 14 (E14), OMgp expression in the brain is undetectable. By embryonic day 18 (E18), OMgp mRNA is detected slightly in the brain. In both the rat brain and spinal cord there seems to be constant expression of OMgp mRNA between birth (P0) and post-natal day 8 (P8) with the maximum level of OMgp mRNA at adulthood. See FIG. 3.

### Example 2

### Creation of OMgp Knock-Out Mouse

OMgp is expressed by oligodendrocytes and neurons. To further investigate the role of OMgp in neuronal and oligodendrocyte function, a mouse which was null for the OMgp locus was created.

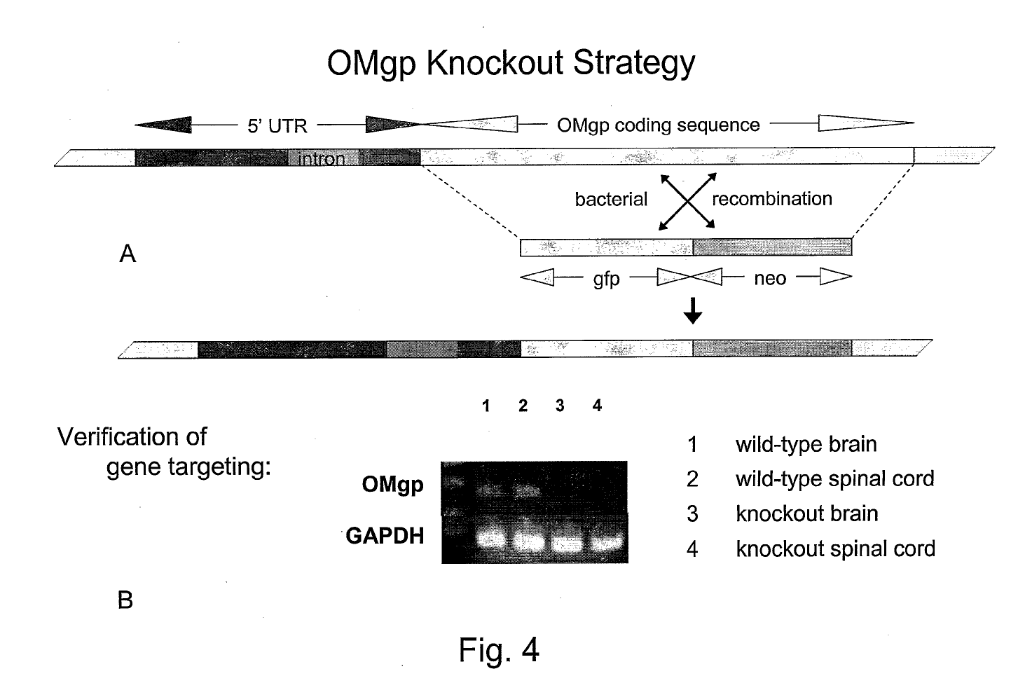

OMgp knock-out mice were generated with a GFP/Neo (green fluorescent protein/neomycin) replacement vector that targeted the entire, single exon coding sequence of OMgp as described by Schiemann et al. (Science 293: 2111-2114 (2001)). Mouse genomic 129/SvJ DNA was isolated from a lambda genomic library (Stratagene # 946313; Stratagene, La Jolla, Calif.). A 9.9 kb NotI-EcoRV fragment was subcloned into pBSK+ (Stratagene, La. Jolla, Calif.), then targeted by homologous recombination in bacteria to insert an eGFP reporter gene and neomycin antibiotic resistance gene at the initiating ATG codon of OMgp. The final construct deleted the entire 1-1299 nt single exon coding sequence of OMgp. See FIG. 4A.

This construct was then used to target the OMgp locus in V6.5 embryonic stem (ES) cells (obtained from R. Jaenisch). Correctly targeted cells were identified by Southern blotting of XbaI digested ES cell DNA and injected into C57B1/6 blastocysts to generate chimeric mice. Chimeras were crossed to C57B1/6 mice to generate heterozygous founder mice.

Genotypes of the mice were determined by three-primer PCR of tail DNA. The forward primer 5′-CCGAATGCTAACTGACCCATGC-3′ (SEQ ID NO:12) and the two reverse primers 5′-GAACAGTCCACATGCCTGTGCC-3′ (SEQ ID NO:13) and 5′-GATGCCCTTCAGCTCGATGCG-3′ (SEQ ID NO:14) yielded a 207 bp wild-type PCR product and a 496 bp mutant allele PCR product in a 35-cycle reaction (94° C. for 20 sec., 65° C. for 30 sec., 72° C. for 30 sec). The OMgp null mice analyzed were of a mixed 129SvJ, C57B1/6 background.

Null mice were then tested for their ability to produce OMgp mRNA from brain and spinal cord samples. RT-PCR was performed as described in Sha et al., Nat. Neurosci. 8:745-51 (2005), using the following primers:

| | |
|---|---|
| 5′ PCR Primer- | |
| 5′-GCCCACAGCCAGAAACAGTT-3′ | (SEQ ID NO:15) |
| 3′ PCR Primer- | |
| 5′-TCCAGGGTCCTCAGATTGGTAT-3′. | (SEQ ID NO:11) |

FIG. 4B shows the results of RT-PCR reactions from the spinal-cord and brain of four separate litter mate mice. Mouse 1 and mouse 2 produced detectable levels of OMgp transcript, while the knock-out mice produced no OMgp in the brain and spinal cord. See FIG. 4B.

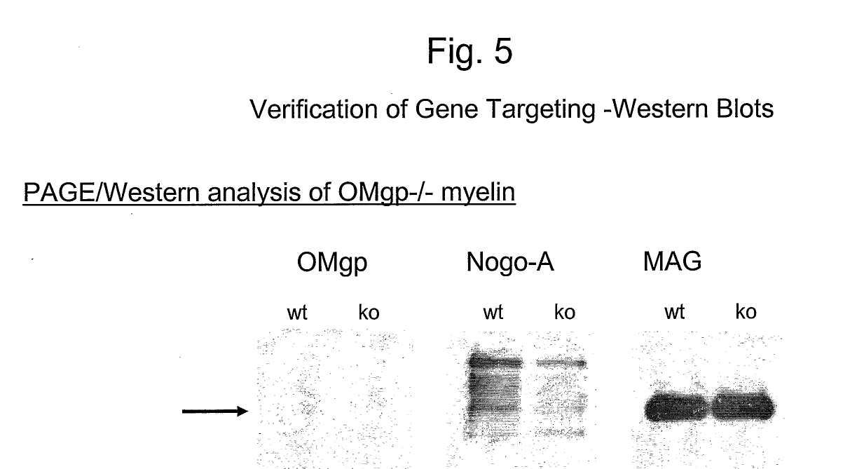

To further verify that OMgp was deleted in the knock-out mice, OMgp, Nogo-A and MAG protein expression was tested via Western blots on wild-type and OMgp null mice. Total protein lysates from brain and spinal cord tissue were run on 4-20% gradient SDS/PAGE gels and then transferred to nitrocellulose using standard procedures. The blots were probed with rabbit polyclonal antibodies to OMgp (Biogen Idec Rabbit Number 223 and 224), Nogo-A (AB5664P, Chemicon International, Inc., Temecula, Calif.) and MAG (OMG (K-21): sc-14524, Santa Cruz Biotechnology, Inc., Santa Cruz, Calif.). Nogo-A and MAG protein were both present in the wild-type and OMgp null mice. See FIG. 5. However, OMgp protein was only detectable in the wild-type mice and not the OMgp knock-out mice, thus confirming that no OMgp is produced in these mice.

### Example 3

### Increased Number of Hippocampal Neurons in OMgp Knock-Out Mice Cell Cultures

OMgp knock-out mice and wild-type mice were sacrificed at day E15 and their brains were removed. Cells from the cortex, hippocampus, and cerebellum were isolated according to the following protocol. Only the hippocampus is referred to in the protocol, but the same protocol was used for isolating other portions of the mouse brain as well. Poly-L-lysine coated coverglasses were placed into 24 well tissue culture dishes and 300 μl of plating medium (2 mM L-glutamine, 1× penicillin-streptomycin, 1×B27, 50 μM glutamateric acid in neurobasal medium) was added to each well. 3-4 ml of S-MEM was added to 60 mm Petri dishes (1 petri dish for two brain) and the Petri dishes were kept on ice. The hippocampi were dissected out of the mouse brain and then minced into small pieces. The minced hippocampi were transferred into a 15 ml tube with 2 ml of S-MEM for every 10 hippocampi. Trypsin was added to a final concentration of 0.1%. The tube was incubated in a 37° C. water bath for 10 minutes with shaking by hand every 3 minutes. DNase I was added to the hippocampi at a final concentration of 0.05 to 0.1 mg/ml. An equal volume of DMEM with 10% FBS was added as well. Hippocampal cells were dissociated by pipeting up and down 10 times with a 10 ml pipette. The tube was allowed to stand for 2-3 minutes to allow undissociated tissue pieces to settle to the bottom of the tube. The supernatant was transferred to a fresh tube and spun at 1100 rpm for 3 minutes to collect all cells. The cells were resuspended in plating medium, counted and 2.5×10⁴ cells were plated into each well of a 24 well tissue culture plate. 0.5 to 1 ml of culture medium (2 mM L-glutamine, 1× penicillin-streptomycin, 1×B27 in neurobasal medium) was added to each well. The hippocampal neurons were grown in culture for 3 days.

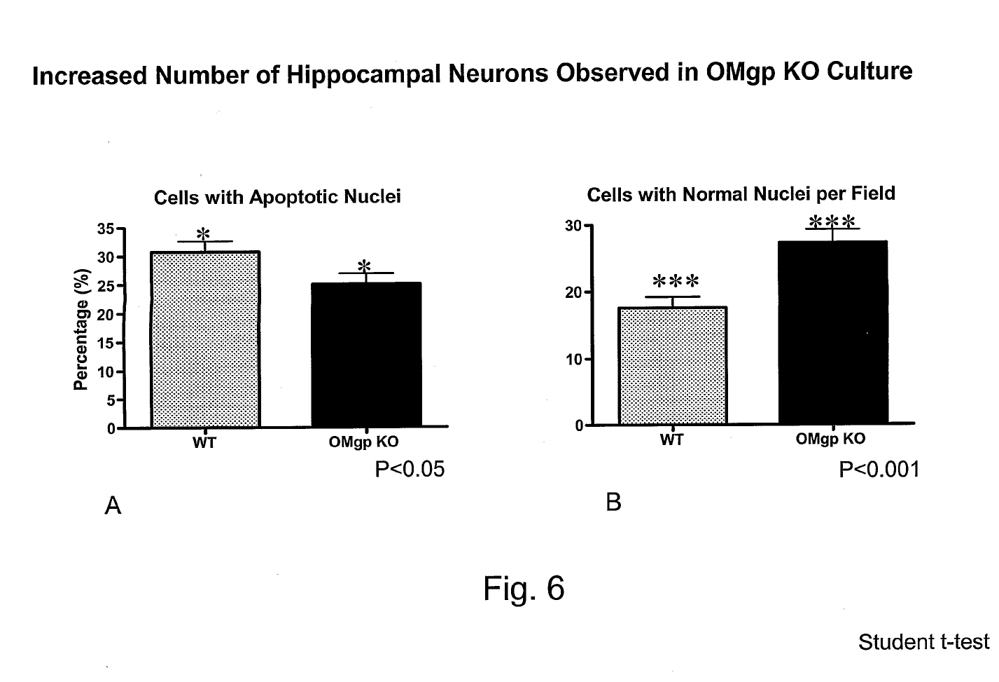

On day three, hippocampal neurons in the cultures from the OMgp knock-out mice were compared to the wild-type hippocampal neuron cultures. Cells were stained with anti-NeuN antibody (Chemicon) (to identify neurons) and DAPI (to stain nuclei) and cells with normal nuclei were counted under a microscope. The wild-type hippocampal cell culture contained fewer cells than the cell cultures from OMgp knock-out mice. The wild-type hippocampal cell cultures contain a little less than about 20 cells with normal nuclei per field while the OMgp knock-out hippocampal cell cultures contained almost about 30 normal cells per field. See FIG. 6B

The same cell cultures were then examined for apoptotic cells. The wild-type and OMgp knock-out hippocampal neuron cultures were stained for cells undergoing apoptosis with the apoptotic marker caspase-3. OMgp hippocampal neuronal cultures had fewer cells under going apoptosis as compared to the wild-type neuronal cultures. About 30% of the wild-type neuronal cultures were undergoing apoptosis at day 3. However, at day 3 only about 25% of the hippocampal neuronal cells in culture were undergoing apoptosis. See FIG. 6A.

Additionally, hippocampal neurons isolated from OMgp knock-out mice also show an increased resistance to glucose deprivation as compared to wild-type mice. These results suggest that OMgp knock-out mice have increased survival in cell culture relative to mice that express the OMgp protein.

### Example 4

### Cultured Hippocampal Neurons from OMgp Knock-Out Mice Display Earlier Neurite Extension

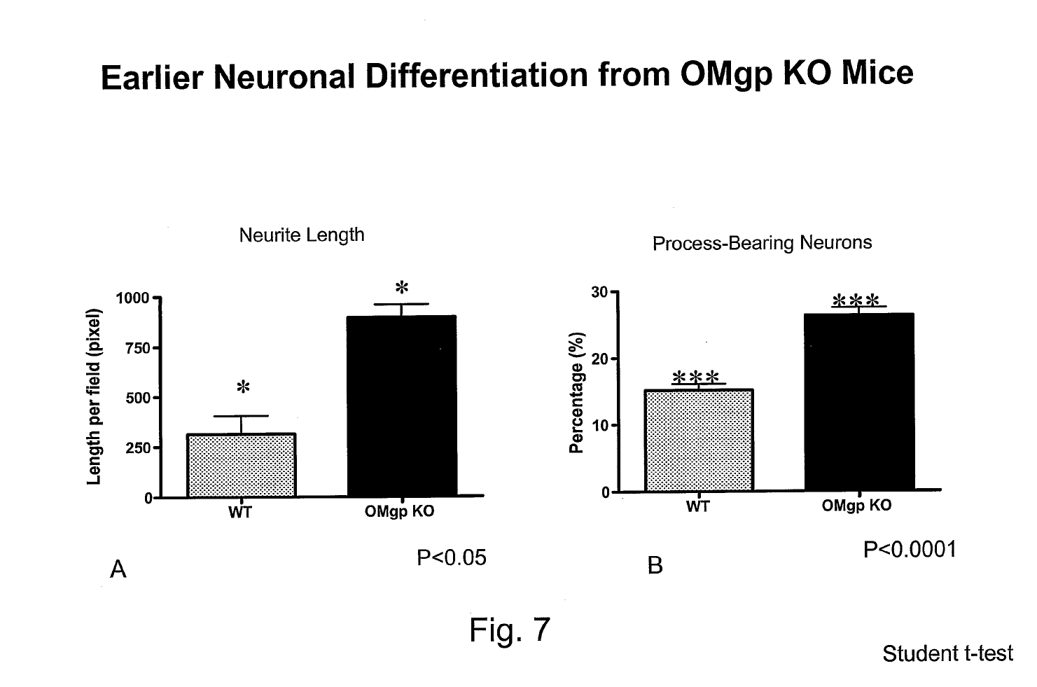

Hippocampal neurons from OMgp knock-out mice and wild-type mice were grown in tissue culture, as described above, for 24 hours and then examined for neurite-length and the number of process-bearing neurons. Cells were stained with DAPI and an anti-β-tubulin III antibody. The length of each neuron was measured, under a microscope, in pixels and then averaged per field. The length of hippocampal neurons from OMgp knock-out mice were over three times the length of neurons from wild-type mice. See FIG. 7A.

Additionally, process bearing neurons were counted in each culture under a microscope. In neuronal cultures from wild-type mice, approximately 15% of the cells were process-bearing. However, in neuronal cultures from OMgp knock-out mice, about 25% of the cells were process-bearing. See FIG. 7B.

These data suggest that neurons from OMgp null mice display an earlier onset of differentiation and that OMgp has a role in inhibiting neuronal differentiation.

### Example 5

### Neurons in OMgp Knock-Out Mice Show Increased Survival after Spinal Cord Injury

Wild-type mice and OMgp knock-out mice were subject to spinal cord injury (SCI) as described. Briefly, a dorsal hemisection was performed at T6/T7, completely interrupting the main dorsomedial and the minor dorsolateral corticospinal tract (CST) components. The cord was stereotaxically transected at a depth of 1.8 mm from the surface using a microscalpel. At the end of the study, mice were anesthetized and trans-cardially perfused with heparinized saline followed by 4% paraformaldehyde (PFA). The spinal cords were removed, embedded in paraffin, and 10 μm sections were cut from for histological analysis.

To quantify apoptotic cell death after SCI, animals were euthanized 28 days after SCI and stained using anti-activated-caspase-3 antibody (Cell Signaling Technologies, Beverly, Mass.). The sections were also stained with anti-NeuN antibody (Chemicon) to identify neurons.

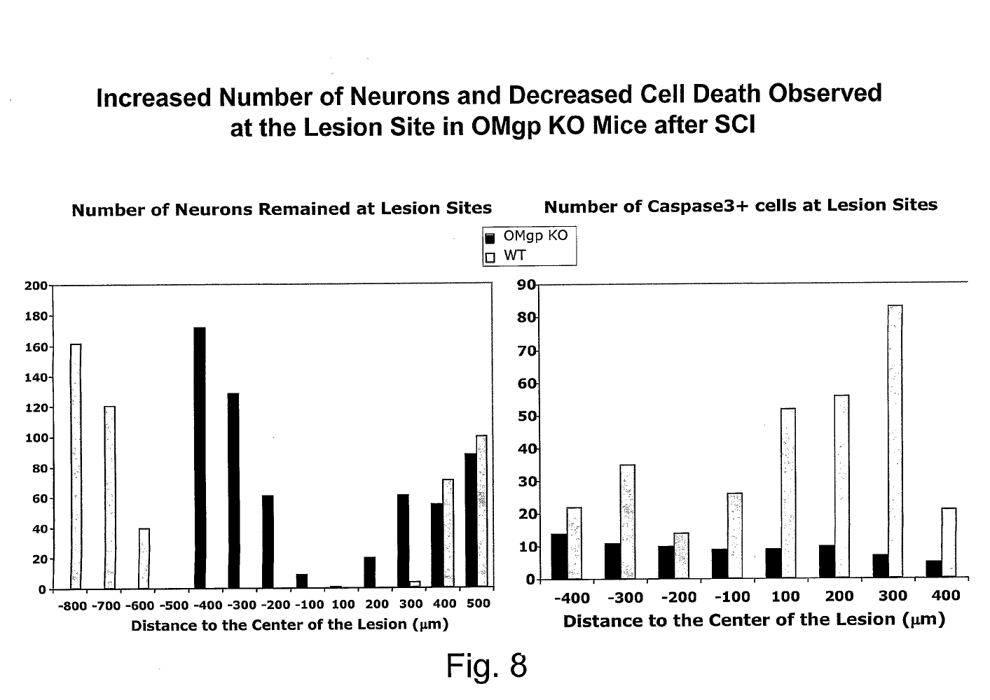

We observed activated-caspase-3 staining both rostral and caudal to the site of transection 3 days after SCI which co-localized with neurons. The number of activated-Caspase-3-positive neurons was significantly smaller in the OMgp knock-out mice as compared to the wild-type mice, 28 days after SCI. The number of caspase-3-positive cells were quantified in both wild-type and OMgp knock spinal cord sections. Cells were counted at various distances from the center of the lesion. As shown in FIG. 8B, the OMgp knock-out mice contained significantly fewer caspase-3-positive cells.

Additionally, the number of neurons at the lesion site were quantified for OMgp knock-out mice and wild-type mice. The sections were also stained with anti-NeuN antibody (Chemicon) to identify neurons and the neurons were counted at various distances away from the center of the spinal cord lesion. The OMgp knock-mice had significantly more neurons relative to the wild-type mice. See FIG. 8A.

These data show that OMgp knock mice have increased neuronal cell survival after SCI as compared to wild-type mice which express OMgp. Together, these data show a role for endogenous OMgp in the inhibition of neuronal survival after SCI.

### Example 6

### Functional Recovery after Spinal Cord Injury in OMgp Knock-Out Mice

Wild-type mice and OMgp knock-out mice were subject to spinal cord injury (SCI). All experiments were performed in 8 week-old female mice from mixed C57BL/6 and 129SvJ strains. All procedures were performed in accordance with the NIH Guide for the Care and Use of Laboratory Animals and approved by the Biogen Idec Institutional Animal Care and Use Committee.

Mice were anesthetized by injection with Ketamine mixed with Xylazine (90 μg Ketamine/9.9 μg Xylazine/kg, i.p.). The hair on the back was shaved and wiped with betadine and 70% ethanol swab (BD Diabetes). A drape (3M Health Care) was used to cover the animal and a midline incision was made over the thoracic vertebrae. The paravertebral muscles were separated from the vertebral column and retracted, a dorsal laminectomy was performed at thoracic vertebra T10. A micro scalpel (150, Electron Microscopy Sciences), was used for the complete transection. Forceps were used to hold the vertebral column in a slightly lifted position and the spinal cord was cut from the dorsal to the ventral surface a few times until clear separation was noted between the rostral and caudal stumps of the spinal cord. See Inman et al., Spinal cord Injury Research Techniques, Reeve-Irvine Research Center (2003).

A small piece of gelfoam was overlaid on the transection. Muscle was then closed by two sutures next to the transection. The skin was then closed with wound clips. All procedures were performed under a surgical microscope. The animal was allowed to recover from anesthesia in a heating chamber. Buprenorphine (0.1 μg/kg, twice a day, sc) was administered for two days. Baytril (Enrofloxacin (2.5 μg/kg, twice a day, sc) was administered for 14 days. The initial doses of Buprenorphine and Baytril were administered pre-operatively. The animal bladders were expressed twice a day until bladder functions returned.

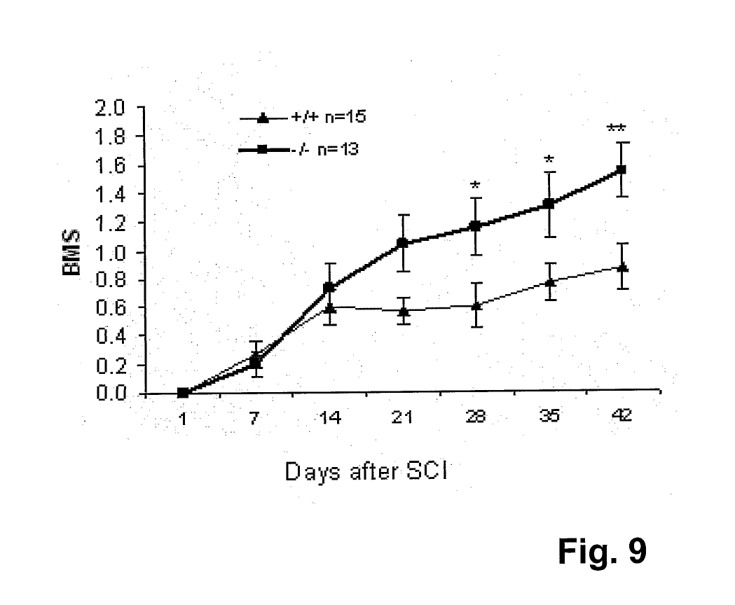

Recovery from SCI injury was quantified using the Basso Mouse Scale (BMS) for locomotion scoring method (See Basso D. M., et al. “The Basso Mouse Scale (BMS) for locomotion defects differences in recovery after spinal cord injury in five common mouse strains.” J. of Neurotrauma (In press) and Basso & Fisher, J. Rehab. Res. Dev. 40:S3, 12 (2003), both of which are incorporated herein by reference). The BMS scale was developed specifically for mice. See Engesser-Cesar et al., J. of Neurotrauma 22:157-171 (2005), which is incorporated herein by reference. The BMS scale is an evaluation of open field locomotor function. The scale is from 0 to 9 with example scores as follows: 0=no ankle movement; 1=Slight ankle movement; 2=extensive ankle movement; 3=Plantar placing of the paw, with or without weight support or occasional, frequent or consistent dorsal stepping but not plantar stepping. A complete description of the BMS scale can be found in Engesser-Cesar et al., J. of Neurotrauma 22:157-171 (2005), which is incorporated herein by reference. OMgp knock-out mice and wild-type mice were monitored for recovery of function after spinal cord injury as demonstrated by an increase in BMS score, and axon regeneration as observed by immunohistochemical staining of the axons. OMgp knock-out mice showed an increase in locomotion following SCI relative to wild-type mice. See FIG. 9.

### Example 7

### Rho Inactivation after Spinal Cord Injury in OMgp Knock-Out Mice

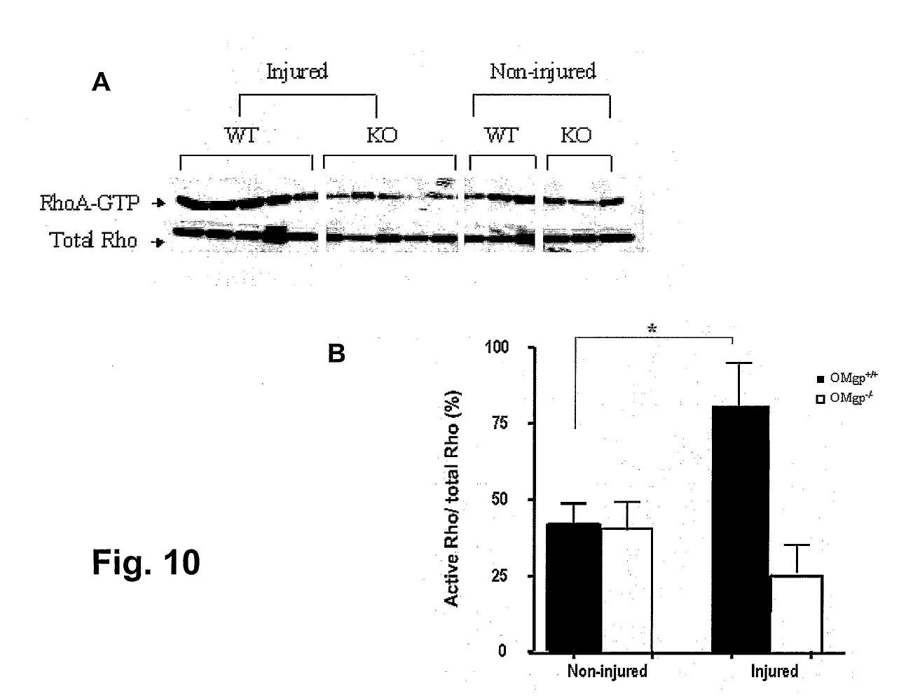

To determine whether OMgp affects the RhoA pathway, RhoA GTP levels in cell lysates of spinal cord segments from OMgp knock-out mice were compared with levels in corresponding spinal cord segments from wild-type mice. The mice were subject to spinal cord injury as described in Example 6 or were uninjured. The levels of RhoA GTP were measured by western blotting. A significant reduction in RhoA GTP was seen in OMgp knock-out mice that had been subjected to SCI. See FIGS. 10A and 10B. These results suggest that attenuation of OMgp function may induce axon regeneration by downregulating RhoA GTP.

### Example 8

### In vitro Oligodendrocyte Survival/Proliferation/Differentiation Assay

Oligodendrocytes mature through several developmental stages from A2B5 progenitor cells (which express A2B5), differentiating into pre-myelinating oligodendrocytes (which express O1 and O4) and finally into mature myelinating oligodendrocytes (which express O1, O4 and MBP). Thus, by monitoring the presence and absence of the A2B5, O1, O4 and MBP markers it is possible to determine a given cell's developmental stage and to further evaluate the role of OMgp in oligodendrocyte biology. For a general review of oligodendrocyte biology, see, e.g., Baumann and Pham-Dinh, Physiol. Rev. 81: 871-927 (2001).

To test the survival, proliferation and differentiation of oligodendrocytes in the presence of various OMgp antagonists, A2B5+ oligodendrocytes are treated with various concentrations of OMgp antagonists or a control for 3 days. For assessing differentiation, A2B5+ cells are plated in 4-well slide chambers in FGF/PDGF-free growth medium supplemented with 10 ng/ml CNTF and 15 nM triiodo-L-thyronine and are immediately treated with increasing concentrations of OMgp antagonists or control. After 48 h (72 h for RNAi), cultures are stained with antibody to O4, and the number of total O4+ and mature O4+ oligodendrocytes are quantified. Samples are analyzed in duplicate.

Mature oligodendrocytes have a half-life in vitro of about 48 to 72 hours, with cells typically undergoing apoptosis after 72 hours. Increased survival rate for mature oligodendrocytes is judged by cell viability staining as compared to control treated cells. MBP expression is also monitored as a marker for mature oligodendrocytes.

### Example 9

### In vitro Neuronal Survival Assay

To test the survival, proliferation and differentiation of neurons in the presence of various OMgp antagonists, neuronal cultures are treated with various concentrations of OMgp antagonists or a control. Equal numbers of rat P6 cerebellar granule neurons are plated in each well of a 12-well cell culture poly D-lysine coated plate in the presence or absence of an OMgp antagonist. The neuronal cultures are maintained for 1-7 days at 37° C. and 5% CO₂. Neuron survival is monitored, after three days, by physical appearance. For example, the presence of a rounded cell body with condensed nuclear material and/or the presence of neurite extensions [determined by the presence of the neuronal specific marker, β-tubulin III]. The number of cells in each well is also counted by staining nuclei wth DAPI.

### Example 10

### Rat Spinal Cord Injury Model

Spinal cord injury (“SCI”) is induced by dorsal over-hemi-section as follows, modified from methods described previously (Li, S. et al. J. Neurosci. 24, 10511-10520 (2004)). Anesthetized female Long Evans rats (7 weeks old, Charles River) are given pre-operative analgesia (Buprenorphine/Buprenex, 0.05 mg/kg s.c.) and tranquillized (Midazolam, 2.5 mg/kg i.p.) and a dorsal hemi-section is performed at thoracic vertebra 6/7 completely interrupting the main dorsomedial and the dorsolateral corticospinal tract (CST). The dorsal and dorso-lateral components of the corticospinal tract (CST) are completely interrupted and the ventral portion of the CST left intact.

Hindlimb function is quantified using the Basso-Beattie-Bresnahan (BBB) open field scoring method (Eby, M. T. et al., J. Biol. Chem. 275, 15336-15342 (2000), incorporated herein by reference). Immediately after CST transection, an intrathecal catheter is inserted into the subarachnoid space at T7 and connected to a primed mini-osmotic pump (Alzet model 2004, Alza Corp) and inserted into the subcutaneous space. Mini-osmotic pumps deliver Human IgG isotype control protein (5 mg/ml) or a monoclonal OMgp antagonist antibody, soluble OMgp polypeptide or an RNAi molecule (˜5 mg/ml) continuously at a rate of 0.25 μl/h over 5 weeks. Rats are monitored for recovery of function after spinal cord injury as demonstrated by an increase in BBB score, and axon regeneration and axon retraction as observed by immunohistochemical staining of the axons.

### Example 11

### In vitro Myelination Assay

The role of anti-OMgp antagonist antibodies, soluble OMgp polypeptides and OMgp antagonist polynucleotides (e.g. RNAi) in myelination is investigated in vitro by treating co-cultures of dorsal root ganglion (DRG) neurons and oligodendrocytes with the various OMgp antagonists individually and testing for myelination by immunohistochemistry and Western blotting. For these studies, it is necessary to first generate primary cultures of DRG neurons and of oligodendrocytes.

Female Long Evans rat E14-E17 embryonic dorsal root ganglia are cultured as described by Plant et al., J. Neurosci. 22:6083-91 (2002). Dissected DRGs are plated on poly-L-lysine-coated cover slips (100 μg/ml) for 2 weeks. The cells are incubated in the presence of fluorodeoxyuridine for days 2-6 and in NLA medium containing 1×B27, 100 ng/ml NGF (Gibco) for days 8-11.

Female Long Evans post-natal day 2 (P2) rat oligodendrocytes are cultured as described by Conn, Meth. Neurosci. 2:1-4 (Academic Press; 1990) with modifications as follows. Briefly, the forebrain is extirpated from P2 rats and placed in cold HBSS medium (Gibco). The tissue fragments are cut into 1 mm pieces and incubated at 37° C. for 15 min in 0.01% trypsin and 10 μg/ml DNase. Dissociated cells are plated on a poly-L-lysine coated T75 tissue culture flasks and grown in DMEM with 20% fetal bovine serum at 37° C. for 10 days. A2B5-positive oligodendrocytes are collected by shaking the flasks overnight at 200 rpm at 37° C. The A2B5 oligodendrocytes are cultured for 7 days in DMEM (Gibco) containing 25 mM D-glucose, 4 mM L-glutamine, 1 mM sodium pyruvate, 50 μg/ml human apo-transferrin, 5 μg/ml bovine pancreatic insulin, 30 nM sodium selenate, 10 nM hydrocortisone, 10 nM D-biotin, 1 mg/ml BSA, 10 ng/ml FGF and PDGF (Peprotech). The cells are then harvested by trypsinization. The cells are then co-cultured with the DRG neurons in the presence or absence of 1, 3, 10, or 30 μg/ml of anti-OMgp monoclonal antibodies, soluble OMgp polypeptide or a negative control antibody, in NLA medium containing 2% fetal bovine serum, 50 μg/ml ascorbic acid, 100 ng/ml NGF (Gibco). The culture medium is changed and the various OMgp antagonists are replenished every three days. After 30 days at 37° C., the co-cultured cells are stained by immunohistochemical staining (“IHC”) for neurofilaments with anti-βIII-tubulin antibody to identify axons, or anti-MBP antibody to identify oligodendrocytes. Co-cultured cells are also lysed and subjected to Western blot analysis to quantify the MBP.

### Example 12

### In Vivo Oligodendrocyte Survival, Neuronal Survival and Myelination Assay Using OMgp Antagonist

Adult wild-type C57B1/6 male mice are fed cuprizone (0.2% milled with ground mouse chow by weight) for 6 weeks to induce demyelination within the corpus callosum according to the method described by Morell P et al., Mol Cell Neurosci. 12:220-7 (1998). Briefly, anti-OMgp monoclonal antagonist antibodies, OMgp soluble polypeptides or OMgp antagonist polynucleotides (e.g. RNAi) are stereotactically injected into the demyelinating corpus callosum at weeks 2, 2.5, and 3 weeks of cuprizone feeding, by the method described below. Control mice are stereotactically injected at the same intervals with sterilized media containing a control. After 6 weeks of cuprizone feeding, the mice are returned to a normal diet for 2, 4 and 6 weeks (ground mouse chow only) to allow for remyelination.

The OMgp antagonist monoclonal antibodies, OMgp soluble polypeptides, OMgp antagonist polynucleotides and control are delivered as follows. The cuprizone-treated mice are anesthetized with ketamine (80 mg/kg body weight) and xylazine (10 mg/kg body weight) and positioned in an immobilization apparatus designed for stereotactic surgery (David Kopf Instruments). The scalp is opened and the sterile compounds are injected (1 μM in 1 ml of HBSS) unilaterally into the acutely demyelinated corpus callosum of the wild-type recipient mice with a 10 ml Hamilton syringe using stereotactic coordinates of 0.7 mm posterior and 0.3 mm lateral to bregma at a depth of 1.7 mm (Messier et al., Pharmacol. Biochem. Behav. 63: 313-18 (1999)). Additional control recipient mice are stereotactically injected with HBSS containing no compounds. The opening in the skull is filled with Gelfoam, and the area is swabbed with penicillin and streptomycin (Gibco) and the wound is sutured. Mice are sacrificed every week of the experiment after injection and their brains are removed and processed for molecular, biochemical and histological analysis.

Oligodendrocytes and neurons from the animals receiving treatment are then examined for increased mature oligodendrocyte survival (based on CC1 antibody staining), neuronal survival (based on NeuN or β-tubulin III staining) and axon myelination by IHC using anti-MBP protein antibody or luxol fast blue.

### Example 13

### In vivo Transplantation of OMgp-Transformed Cells

To investigate the biological function of OMgp in spinal cord injury, cortical primary cultured cells (mixed cultures) are infected with retrovirus expressing OMgp or a retrovirus control, for delivery into the injured epicenter of rat spinal cords. 2×10⁶ cells are introduced, and the rats are sacrificed at day 10. The spinal cords are fixed in 4% paraformaldehyde overnight, then dehydrated in 70% ethanol, followed by 95% ethanol. Tissue samples are imbedded in paraffin. Sections (10 microns thick) are used for immunohistochemical staining. We monitor oligodendrocyte survival and axon myelination in the injured rats receiving OMgp.

### Example 14

### OMgp-Specific RNAi Knockdown of OMgp Expression

OMgp-specific RNAi is used to ablate OMgp expression in oligodendrocyte precursor cells to examine how OMgp contributes to oligodendrocyte growth and differentiation. 50,000 A2B5 oligodendrocyte precursor cells are infected with lentivirus carrying OMgp-specific RNAi sequence or control RNAi prepared as follows.

OMgp DNA sequences from various species are compared to find homologous regions to use for candidate small-hairpin RNAs (shRNA). Constructs for lentivirus expression of OMgp RNAi, are constructed using methods known in the art and the pLL3.7 vector. The pLL3.7 vector, additional methodology and virus production is described in Rubinson et al., Nat. Genet. 33, 401-06 (2003).

Prior to producing the lentivirus, DNA from pLL3.7, or candidate shRNA in pLL3.7, are cotransfected with a murine OMgp-HA tagged plasmid, at a ratio of 5 to 1, into CHO cells in 6-well format. Knockdown is analyzed by western blot detection of OMgp-HA tag from transfected CHO cell lysates as well as by northern blot of total RNA prepared from duplicate wells. The blots are probed with a fragment of OMgp cDNA. Assays are performed 48 hours post-transfection.

RNAi lentiviruses carrying green fluorescent protein (GFP) are generated as described in Rubinson et al. In cultures treated with either control or OMgp RNAi. To quantify the effects of RNAi on differentiation, only GFP-expressing oligodendrocytes are counted.

Enriched populations of oligodendrocytes are grown from female Long Evans P2 rats as described by Conn, Meth. Neurosci. 2:1-4 (Academic Press; 1990) with modifications as follows. Briefly, the forebrain is dissected and placed in Hank's buffered salt solution (HBSS; Invitrogen). The tissue is cut into 1-mm fragments and is incubated at 37° C. for 15 min in 0.01% trypsin and 10 μg/ml DNase. Dissociated cells are plated on poly-L-lysine-coated T75 tissue culture flasks and are grown at 37° C. for 10 d in DMEM medium with 20% fetal calf serum (Invitrogen). Oligodendrocyte precursors (A2B5+) are collected by shaking the flask overnight at 200 rpm at 37° C., resulting in a 95% pure population. Cultures are maintained in high-glucose Dulbecco's modified Eagle's medium (DMEM) with FGF/PDGF (10 ng/ml; Peprotech) for 1 week. Removal of FGF/PDGF allows A2B5+ cells to differentiate into O4+ premyelinating oligodendrocytes after 3-7 d, and to differentiate into O4+ and MBP+ mature oligodendrocytes after 7-10 d. These differentiation states are readily apparent from changes in morphology: A2B5+ cells are bipolar in shape, O4+ premyelinating oligodendrocytes have longer and more branched processes and MBP+ mature oligodendrocytes contain myelin sheet structures between processes.

A2B5 oligodendrocyte precursor cells are infected with the lentivirus containing the OMgp RNAi. The resulting cells are cultured for 3 days and the number of O4-positive (a marker for oligodendrocyte differentiation) oligodendrocytes is counted. Endogenous OMgp expression is reduced by infection with OMgp RNAi lentivirus and is confirmed by RT-PCR.

### Example 15

### Production of OMgp-Specific Monoclonal Antibodies

Anti-OMgp Antibodies that specifically bind an immunogenic OMgp polypeptide of the invention are made using the following methods and procedures.

### A. Antibody Screening Assays

1. ELISA Assay

OMgp-Fc (0.5 μg in 50 μl of 0.1 M sodium bicarbonate buffer, pH 9.0) is added to each well of a 96-well MaxiSorp™ plate (Nunc™). The plates are then incubated at 37° C. for 1 hour or 4° C. for 16 hours. Non-specific binding sites on the plates are blocked using 25 mM HEPES, pH 7.4 containing 0.1% BSA, 0.1% ovalbumin, 0.1% (5% (w/v) nonfat dry milk in 150mM NACE) and 0.001% azide. Dilutions of serum or hybridoma supernatants (for example, serial three-fold dilutions) are added across each row of the plate, and incubated at 25° C. for 1 hour. After washing three times with PBS, 50 μl of a 1:10,000 dilution of horseradish peroxidase-conjugated goat anti-mouse secondary antibody (Jackson ImmunoResearch Inc.) is added to each well and incubated further for 1 hour. After three washings, color was developed by TMB (Pierce) and stopped with 2 M sulfuric acid. Color intensity is monitored in a spectrophotometer at 450 nm.

2. FACS Assay

COS-7 cells are labeled with 0.1 μM CellTracker™ Green CMFDA (Molecular Probes, Eugene, Oreg.) as described by the vendor. Equal volumes of CellTracker™ labeled control cells are mixed with washed OMgp-COS-7 cells (produced by transient transfection of an OMgp expression vector which expresses full-length OMgp or a fragment thereof) before incubation with anti-OMgp test sera or hybridoma supernatants. Fifty microliters of the cell mixture is dispensed into each well of a 96-well V-bottom polystyrene plate (Costar® 3877, Corning, N.Y.) and 100 μl of mouse serum, hybridoma supernatant, or a control anti-OMgp antibody is added. After incubation at 4° C. for 30 minutes, the cells are washed and incubated with 50 μl of phycoerythrin-conjugated affinity pure F(ab′)₂ fragment goat anti-mouse IgG Fc gamma specific second antibody (1:200, Jackson ImmunoResearch Laboratory, West Grove, Pa.) in PBS. At the end of the incubation, the cells are washed twice with PBS and suspended in 200 μl of PBS containing 1% fetal bovine serum (FBS), and further subject to FACS analyses. Alternately, OMgp-COS-7 cells are mixed with mouse serum or hybridoma supernatant and then treatment with R-phycoerythrin-conjugated goat anti-mouse secondary antibody followed by standard FACS analyses.

### B. Hybridoma Production of Murine Monoclonal Anti-OMgp Antibodies

Eight-week-old female RBF mice (Jackson Labs, Bar Harbor, Me.) are immunized intraperitoneally with emulsion containing 50 μg OMgp-Fc or are immunized intraperitoneally with an emulsion containing 50 μg of OMgp-Fc, and 50 μl complete Freund's adjuvant (Sigma® Chemical Co., St. Louis, Mo.) once every two weeks.

Additionally, the OMgp knock-out mice, as described in Example 2, can be immunized to produce anti-OMgp antibodies. Sera from the immunized mice are collected before the first immunization and 1 week after the second and third immunizations, and anti-OMgp antibody titers are measured by FACS assay on OMgp-expressing COS-7 cells as described above. A booster final dose is given after the third immunization and three days prior to when hybridoma fusions are initiated.

Sera from mice immunized with the various OMgp peptides are screened by ELISA as described above. Mice that are positive for antibodies that specifically bind OMgp expressing COS-7 cells are identified by flow cytometry (FACS) as described above, and are sacrificed. Splenocytes are isolated from the mice and fused to the FL653 myeloma (an APRT-derivative of a Ig-/HGPRT-Balb/c mouse myeloma, maintained in DMEM containing 10% FBS, 4500 mg/L glucose, 4 mM L-glutamine, and 20 mg/ml 8-azaguanine) as described in Monoclonal Antibodies. Hybridomas: A New Dimension in Biological Analyses, ed. Kennett, R. H., McKearn, T. J. and Bechtol, K.B. New York: Plenum Press (1982). Fused cells are plated into 24- or 48-well plates (Corning Glass Works, Corning, N.Y.), and fed with adenine, aminopterin and thymidine (AAT, available from Sigma® Chemical Co., St. Louis, Mo.) containing culture medium. AAT resistant cultures are screened by ELISA or flow cytometry as described above for binding to either OMgp-COS-7 cells or to OMgp-Fc. Positive hybridomas are further subcloned by limiting dilution.

Hybridoma cell lines producing monoclonal antibodies produced from mice immunized with OMgp-Fc are isolated. Polynucleotides encoding the variable domains (VH and VL) of certain monoclonal antibodies are isolated by PCR, cloned and are subjected to sequence analysis by the following method. Total RNA is extracted from hybridoma cells using Qiagen® RNeasy® mini kit and cDNA is generated from the isolated RNA by RT-PCR, using standard conditions. A cocktail of primers is used for the RT-PCR. A preferred set of primers include a primer with the 5′ of the primer hybridizing to the signal sequence and the 3′ end of the primer hybridizing to the constant domain 3′ of the FR4/constant domain junction. This allows for the amplification of an intact variable domain with no ambiguities about the monoclonal antibody N-terminus and the V/C junction. One of skill in the art will recognize that primer sets need to be modified for amplifying different templates and for different PCR conditions. Occasionally, the presence of highly abundant nonproductive messages (e.g. the CDR3-FR4 frameshifted nonproductive light chain from the fusion partner) or nonspecific productive messages can be produced and complicate the cloning of variable chains. One solution is to use N-terminal sequence data from the authentic purified antibody to design a degenerate primer to enable cloning. Alternatively, one can use “universal framework” primers, such as those described in Orlandi et al., PNAS 86:3833 (1989), which “fix” the N- and C-termini of the variable domains (i.e. the N-terminus of FR1 and the C-terminus of FR4 are primer-determined).

Additionally, sequence data, for designing more effective primers, can be obtained from the bulk RT-PCR products which have been gel purified and then sequenced. The PCR product can also be subcloned using, for example, the TOPO Cloning Kit (Invitrogen) then sequenced. Sequence data is obtained from multiple independent subclones or gel purified fragments to firmly establish the consensus sequence.

In order to verify that the heavy chain variable domain N- and C-termini are authentic and not primer-determined, another PCR reaction is performed with a degenerate signal sequence primer and a constant domain 3′ primer. The design of the degenerate signal sequence primer is based upon signal sequences of the best hits derived from a TFASTA search of the Genbank rodent sequence database queried with a consensus deduced FR1 sequence from the PCR reaction with the “universal framework primer” described above. This PCR will yield a product with a complete murine heavy chain variable domain.

Complete murine variable domains, identified as described above, are used (with silent mutagenesis as necessary to introduce restriction sites) in conjunction with BIIB human IgG1 and kappa constant domain cDNAs to construct chimeric heavy and light chain cDNAs, respectively. The full-length immunoglobulin cDNAs are then subcloned into a BIIB expression vector called pNE001, a derivative of the commercial EBV mammalian cell episomal expression vector pCEP4. The heavy and light chain expression vectors are co-transfected into 293-EBNA cells. Western blot analysis (probed with human IgG-specific reagents) of conditioned medium from transiently transfected cells will confirm the expression of chimeric OMgp-huIgG1, kappa mAb.

### C. Identification of Anti-OMgp Monoclonal Antibodies by Phage Display

Anti-OMgp monoclonal antibody Fab fragments can be identified and isolated from phage display libraries as described in Hoet et al., Nat. Biotech. 23:344-348 (2005); Rauchenberger, et al., J. Biol. Chem. 278:194-205 (2003); and Knappik, et al., J. Mol. Biol. 296:57-86 (2000), all of which are incorporated herein by reference in their entireties.

The MorphoSys Fab-phage display library HuCAL® GOLD, comprises humanized synthetic antibody variable regions and is screened against recombinant human soluble OMgp-Fc protein by standard ELISA AND IHC screening methods. See, e.g., Ostendorp, R., Frisch, C. and Urban M, “Generation, engineering and production of human antibodies using HuCAL®.” Antibodies, Volume 2 Novel Technologies and Therapeutic Use. New York: Kluwer Academic/Plenum 13-52 (2004). Fab-phages that specifically bind to OMgp are purified and characterized. Isolated Fab-phages are selected for further analysis.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

All publications and patent applications mentioned in this specification are herein incorporated by reference to the same extent as if each individual publication or patent application was specifically and individually indicated to be incorporated by reference.

1. A method for promoting differentiation or survival of a neuron comprising contacting said neuron with an effective amount of an OMgp antagonist selected from the group consisting of:
(i) a soluble OMgp polypeptide;
(ii) an OMgp antibody or fragment thereof;
(iii) an OMgp antagonist polynucleotide, and
(iv) a combination of two or more of said OMgp antagonists of (i) to (iii).

2. A method for promoting differentiation or survival of an oligodendrocyte comprising contacting said oligodendrocytes with an OMgp antagonist selected from the group consisting of:
(i) a soluble OMgp polypeptide;
(ii) an OMgp antibody or fragment thereof;
(iii) an OMgp antagonist polynucleotide, and
(iv) a combination of two or more of said OMgp antagonists of (i) to (iii).

3. A method for promoting myelination of neurons in a mammal, comprising administering to a mammal in need thereof an effective amount of an OMgp antagonist selected from the group consisting of:
(i) a soluble OMgp polypeptide;
(ii) an OMgp antibody or fragment thereof;
(iii) an OMgp antagonist polynucleotide, and
(iv) a combination of two or more of said OMgp antagonists of (i) to (iii).

4. A method for treating a disease, disorder, or injury associated with neuronal death or lack of neuronal differentiation in a mammal comprising administering to a mammal in need thereof a therapeutically effective amount of a composition comprising an OMgp antagonist selected from the group consisting of:
(i) a soluble OMgp polypeptide;
(ii) an OMgp antibody or fragment thereof;
(iii) an OMgp antagonist polynucleotide, and
(iv) a combination of two or more of said OMgp antagonists of (i) to (iii).

5. A method for treating a disease, disorder, or injury associated with dysmyelination or demyelination in a mammal comprising administering a therapeutically effective amount of a composition comprising an OMgp antagonist selected from the group consisting of:
(i) a soluble OMgp polypeptide;
(ii) an OMgp antibody;
(iii) an OMgp antagonist polynucleotide, and
(iv) a combination of two or more of said OMgp antagonists of (i) to (iii).

6. The method of any one of claims 1 to 5, wherein said OMgp antagonist comprises a soluble OMgp polypeptide.

7. (canceled)

8. The method of claim 6, wherein said soluble OMgp polypeptide lacks an OMgp domain selected from the group consisting of:
(i) an OMgp cysteine-rich domain,
(ii) an OMgp LRR domain;
(iii) an OMgp serine/threonine-rich domain; and
(iv) a combination of at least two of said OMgp domains of (i) to (iii).

14. The method of claim 6, wherein said soluble OMgp polypeptide is a cyclic peptide.

15-17. (canceled)

18. The method of claim 6, wherein said soluble OMgp polypeptide further comprises a non-OMgp moiety.

19-28. (canceled)

29. The method of any one of claims 1 to 5, wherein said OMgp antagonist comprises an OMgp antibody, or fragment thereof.

30-32. (canceled)

33. The method of any one of claims 1 to 5, wherein said OMgp antagonist comprises an OMgp antagonist polynucleotide.

34-38. (canceled)

39. The method of any one of claims 3 to 5, wherein said mammal has been diagnosed with a disease, disorder, or injury involving demyelination, dysmyelination, or neurodegeneration.

40-44. (canceled)

45. The method of any one of claims 1 or 2, comprising (a) transfecting said neurons or oligodendrocytes with a polynucleotide which encodes said OMgp antagonist through operable linkage to an expression control sequence, and (b) allowing expression of said OMgp antagonist.

46. The method of any one of claims 3 to 5, comprising (a) administering to said mammal a polynucleotide which encodes said OMgp antagonist through operable linkage to an expression control sequence, and (b) allowing expression of said OMgp antagonist.

47-48. (canceled)

49. The method of any one of claims 3 to 5, wherein said administering comprises (a) providing a cultured host cell comprising said polynucleotide, wherein said cultured host cell expresses said OMgp antagonist; and (b) introducing said cultured host cell into said mammal such that said OMgp antagonist is expressed in said mammal.

50-52. (canceled)

53. The method of any one of claims 3 to 5, wherein said OMgp antagonist is expressed in an amount sufficient to reduce inhibition of neuronal or oligodendrocyte differentiation at or near the site of the nervous system disease, disorder, or injury.

54. The method of any one of claims 3 to 5, wherein said OMgp antagonist is expressed in an amount sufficient to reduce inhibition of neuron myelination at or near the site of the nervous system disease, disorder, or injury.

55-58. (canceled)


SEQUENCE LISTING (93 sequences)

SEQ ID NO: 1 - DNA, 1756 bp, Homo sapiens
gttgaagacgacaccacggctttgatggaatatcagatattgaaaatgtctctctgcctgttcatccttctgtttctcacacctggtattttatgcatttgtcctctccaatgtatatgcacagagaggcacaggcatgtggactgttcaggcagaaacttgtctacattaccatctggactgcaagagaatattatacatttaaacctgtcttataaccactttactgatctgcataaccagttaacccaatataccaatctgaggaccctggacatttcaaacaacaggcttgaaagcctgcctgctcacttacctcggtctctgtggaacatgtctgctgctaacaacaacattaaacttcttgacaaatctgatactgcttatcagtggaatcttaaatatctggatgtttctaagaacatgctggaaaaggttgtcctcattaaaaatacactaagaagtctcgaggttctcaacctcagtagtaacaaactttggacagttccaaccaacatgccctccaaactacatatcgtggacctgtctaataattctttgacacaaattcttccaggtacattaataaacctgacaaatctcacacatctttacctgcacaacaataagttcacattcattccagaccaatcttttgaccaactctttcagttgcaagagataaccctttacaataacaggtggtcatgtgaccacaaacaaaacattacttacttactgaagtggatgatggaaacaaaagcccatgtgatagggactccatgttctacccaaatatcatctttaaaggaacataacatgtatcccacaccttctggatttacctcaagcttattcactgtaagtgggatgcagacagtggacaccattaactctctgagtgtggtaactcaacccaaagtgaccaaaatacccaaacaatatcgaacaaaggaaacaacgtttggtgccactctaagcaaagacaccacctttactagcactgataaggcttttgtgccctatccagaagatacatccacagagactatcaattcacatgaagcagcagctgcaactctaactattcatctccaagatggaatggtcacaaacacaagcctcactagctcaacaaaatcatccccaacacccatgaccctaagtatcactagtggcatgccaaataatttctctgaaatgcctcaacaaagcacaacccttaacttatggagggaagagacaaccacaaatgtaaagactccattaccttctgtggcaaatgcttggaaagtaaatgcttcatttctcttattgctcaatgttgtggtcatgctggctgtctgagggtctgcattttctgaaactaatgaaagcactcctccctgatgtacagttgggaaaatatgtccatatctaaccagtgattcgagctatatttaagtattcaagaaagccagtcttaacatttctaactctgatgtaaatgaagtaacttgtcttaaataaaagaaatgcacaatgtcttggtacttgctgctattttactgtcttaattaagtaaactaatgagtttcttttataaaaaaaatgaaatgttttaaggcttcaatttattgcacaaaatataaagcatctaaactttaatatgtattttatgtatgtttacactgtcaaacatctggaaaataaaaggtctatgctcaaaaaaaaaaaaaaaaaaaaaaaaaaaaaaaaaaaaaaaaaaaaaaaaaaa

SEQ ID NO: 2 - protein, 440 aa, Homo sapiens
MEYQILKMSLCLFILLFLTPGILCICPLQCICTERHRHVDCSGRNLSTLPSGLQENIIHLNLSYNHFTDLHNQLTQYTNLRTLDISNNRLESLPAHLPRSLWNMSAANNNIKLLDKSDTAYQWNLKYLDVSKNMLEKVVLIKNTLRSLEVLNLSSNKLWTVPTNMPSKLHIVDLSNNSLTQILPGTLINLTNLTHLYLHNNKFTFIPDQSFDQLFQLQEITLYNNRWSCDHKQNITYLLKWMMETKAHVIGTPCSTQISSLKEHNMYPTPSGFTSSLFTVSGMQTVDTINSLSVVTQPKVTKIPKQYRTKETTFGATLSKDTTFTSTDKAFVPYPEDTSTETINSHEAAAATLTIHLQDGMVTNTSLTSSTKSSPTPMTLSITSGMPNNFSEMPQQSTTLNLWREETTTNVKTPLPSVANAWKVNASFLLLLNVVVMLAV

SEQ ID NO: 3 - DNA, 2973 bp, Homo sapiens
  misc_feature (1275)..(1275): n can be any nucleotide a, t, g or c
tgaggcaaatgttaatgaggcaatgttaaatatggacccaatgtcagacaaatacatagaaaggagtaagggccaactctcatgcataaggtatcccatcctatagcaaatcagatatataggtacgcttgatgccacaaattttttaaaaaattgtccattttgttgcgtgtgcacctcttgccataaatttgagtcagcaccagcgacagctctgcagtcctcctatgtggtactgatcaggtggttgcagagcttcagctcacagcaacacaatgcagctgagcaggcaagcacagcccacagccagaaacagttccgactctacagaacaagacgacctttaagtttcccagagaaaatgagatgctgatgttgaagacgacaccacgggtaagatgttatttaaatcagtaaaaggctgactttggaatctttttcctttttcttttaagaaaaagtcaacgttaggattaaatatatattcaatagcaagtgcatgcaccagaaatttgctgcagtgtcagttgagggatattttttatacattcagtcactctgtaaatatacatattgttttcctttaaaatgggcactgaaatatacagaaaaaaatcactttataaaatgtgaggtttataggtactgtgttggtctggatttttcaagtgctttttacaaagatatatttatcctaaaaacatacagataaaaatttcgaagactgctttaatatctaaataaaatctaccctatatacacacattgaattacattacctgcagagattaaaaaaaaaagacacgacagccatttttctcatctgagtaagaaagcatatcatcaaaaatagtaatagcctacaactgcaactatttatttgcaaagaatgctattttatcatattaaggctctagaaagataaataagaaagaatatggttagaaaaggggggagggagagagaaaataaaggagaaaatgcaggagagagtagggagagagtctctctctaccacatagcccaatgaaggattaagcattgactataaatgaagggagctttgttagtttaatcactggaacaattataaaaggactcgacaacaacgaggtttattgaaaattttgcctaatgctaactgacccatgcagatgcctaaactgtatttgcatattaaaagaagggtgtatctgtttgtttctaggctttgatggaatatcagatattgaaaatgtctctctgcctgttcatccttctgtttctcacacctgntattttatgcatttgtcctctccaatgtatatgcacagagaggcacaggcatgtggactgttcaggcagaaacttgtctacattaccatctggactgcaagagaatattatacatttaaacctgtcttataaccactttactgatctgcataaccagttaacccaatataccaatctgaggaccctggacatttcaaacaacaggcttgaaagcctgcctgctcacttacctcggtctctgtggaacatgtctgctgctaacaacaacattaaacttcttgacaaatctgatactgcttatcagtggaatcttaaatatctggatgtttctaagaacatgctggaaaaggttgtcctcattaaaaatacactaagaagtctcgaggttctcaacctcagtagtaacaaactttggacagttccaaccaacatgccctccaaactacatatcgtggacctgtctaataattctttgacacaaattcttccaggtacattaataaacctgacaaatctcacacatctttacctgcacaacaataagttcacattcattccagaccaatcttttgaccaactctttcagttgcaagagataaccctttacaataacaggtggtcatgtgaccacaaacaaaacattacttacttactgaagtggatgatggaaacaaaagcccatgtgatagggactccatgttctacccaaatatcatctttaaaggaacataacatgtatcccacaccttctggatttacctcaagcttattcactgtaagtgggatgcagacagtggacaccattaactctctgagtgtggtaactcaacccaaagtgaccaaaatacccaaacaatatcgaacaaaggaaacaacgtttggtgccactctaagcaaagacaccacctttactagcactgataaggcttttgtgccctatccagaagatacatccacagagactatcaattcacatgaagcagcagctgcaactctaactattcatctccaagatggaatggtcacaaacacaagcctcactagctcaacaaaatcatccccaacacccatgaccctaagtatcactagtggcatgccaaataatttctctgaaatgcctcaacaaagcacaacccttaacttatggagggaagagacaaccacaaatgtaaagactccattaccttctgtggcaaatgcttggaaagtaaatgcttcatttctcttattgctcaatgttgtggtcatgctggctgtctgagggtctgcattttctgaaactaatgaaagcactcctccctgatgtacagttgggaaaatatgtccatatctaaccagtgattcgagctatatttaagtattcaagaaagccagtcttaacatttctaactctgatgtaaatgaagtaacttgtcttaaataaaagaaatgcacaatgtcttggtacttgctgctattttactgtcttaattaagtaaactaatgagtttcttttataaaaaaaatgaaatgttttaaggcttcaatttattgcacaaaatataaagcatctaaactttaatatgtattttatgtatgtttacactgtcaaacatctggaaaataaaaggtctatgctcataactgtgtcatttggctttccagtcataccaactttnagcagaatcaaaatgacctcaccatttttgttctagggat

SEQ ID NO: 4 - protein, 440 aa, Homo sapiens
  MISC_FEATURE (21)..(21): Xaa can be any naturally occuring amino acid
MEYQILKMSLCLFILLFLTPXILCICPLQCICTERHRHVDCSGRNLSTLPSGLQENIIHLNLSYNHFTDLHNQLTQYTNLRTLDISNNRLESLPAHLPRSLWNMSAANNNIKLLDKSDTAYQWNLKYLDVSKNMLEKVVLIKNTLRSLEVLNLSSNKLWTVPTNMPSKLHIVDLSNNSLTQILPGTLINLTNLTHLYLHNNKFTFIPDQSFDQLFQLQEITLYNNRWSCDHKQNITYLLKWMMETKAHVIGTPCSTQISSLKEHNMYPTPSGFTSSLFTVSGMQTVDTINSLSVVTQPKVTKIPKQYRTKETTFGATLSKDTTFTSTDKAFVPYPEDTSTETINSHEAAAATLTIHLQDGMVTNTSLTSSTKSSPTPMTLSITSGMPNNFSEMPQQSTTLNLWREETTTNVKTPLPSVANAWKVNASFLLLLNVVVMLAV

SEQ ID NO: 5 - DNA, 1808 bp, Homo sapiens
  misc_feature (145)..(145): n can be any nucleotide a, t, g or c
gttccgactctacagaacaagacgacctttaagtttcccagagaaaatgagatgctgatgttgaagacgacaccacggctttgatggaatatcagatattgaaaatgtctctctgcctgttcatccttctgtttctcacacctgntattttatgcatttgtcctctccaatgtatatgcacagagaggcacaggcatgtggactgttcaggcagaaacttgtctacattaccatctggactgcaagagaatattatacatttaaacctgtcttataaccactttactgatctgcataaccagttaacccaatataccaatctgaggaccctggacatttcaaacaacaggcttgaaagcctgcctgctcacttacctcggtctctgtggaacatgtctgctgctaacaacaacattaaacttcttgacaaatctgatactgcttatcagtggaatcttaaatatctggatgtttctaagaacatgctggaaaaggttgtcctcattaaaaatacactaagaagtctcgaggttctcaacctcagtagtaacaaactttggacagttccaaccaacatgccctccaaactacatatcgtggacctgtctaataattctttgacacaaattcttccaggtacattaataaacctgacaaatctcacacatctttacctgcacaacaataagttcacattcattccagaccaatcttttgaccaactctttcagttgcaagagataaccctttacaataacaggtggtcatgtgaccacaaacaaaacattacttacttactgaagtggatgatggaaacaaaagcccatgtgatagggactccatgttctacccaaatatcatctttaaaggaacataacatgtatcccacaccttctggatttacctcaagcttattcactgtaagtgggatgcagacagtggacaccattaactctctgagtgtggtaactcaacccaaagtgaccaaaatacccaaacaatatcgaacaaaggaaacaacgtttggtgccactctaagcaaagacaccacctttactagcactgataaggcttttgtgccctatccagaagatacatccacagagactatcaattcacatgaagcagcagctgcaactctaactattcatctccaagatggaatggtcacaaacacaagcctcactagctcaacaaaatcatccccaacacccatgaccctaagtatcactagtggcatgccaaataatttctctgaaatgcctcaacaaagcacaacccttaacttatggagggaagagacaaccacaaatgtaaagactccattaccttctgtggcaaatgcttggaaagtaaatgcttcatttctcttattgctcaatgttgtggtcatgctggctgtctgagggtctgcattttctgaaactaatgaaagcactcctccctgatgtacagttgggaaaatatgtccatatctaaccagtgattcgagctatatttaagtattcaagaaagccagtcttaacatttctaactctgatgtaaatgaagtaacttgtcttaaataaaagaaatgcacaatgtcttggtacttgctgctattttactgtcttaattaagtaaactaatgagtttcttttataaaaaaaatgaaatgttttaaggcttcaatttattgcacaaaatataaagcatctaaactttaatatgtattttatgtatgtttacactgtcaaacatctggaaaataaaaggtctatgctcaaaaaaaaaaaaaaaaaaaaaaaaaaaaaaaaaaaaaaaaaaaa

SEQ ID NO: 6 - protein, 433 aa, Homo sapiens
  MISC_FEATURE (14)..(14): Xaa can be any naturally occuring amino acid
MSLCLFILLFLTPXILCICPLQCICTERHRHVDCSGRNLSTLPSGLQENIIHLNLSYNHFTDLHNQLTQYTNLRTLDISNNRLESLPAHLPRSLWNMSAANNNIKLLDKSDTAYQWNLKYLDVSKNMLEKVVLIKNTLRSLEVLNLSSNKLWTVPTNMPSKLHIVDLSNNSLTQILPGTLINLTNLTHLYLHNNKFTFIPDQSFDQLFQLQEITLYNNRWSCDHKQNITYLLKWMMETKAHVIGTPCSTQISSLKEHNMYPTPSGFTSSLFTVSGMQTVDTINSLSVVTQPKVTKIPKQYRTKETTFGATLSKDTTFTSTDKAFVPYPEDTSTETINSHEAAAATLTIHLQDGMVTNTSLTSSTKSSPTPMTLSITSGMPNNFSEMPQQSTTLNLWREETTTNVKTPLPSVANAWKVNASFLLLLNVVVMLAV

SEQ ID NO: 7 - DNA, 1805 bp, Homo sapiens
aatgcagctgagcaggcaagcacagcccacagccagaaacagttccgactctacagaacaagacgacctttaagtttcccagagaaaatgagatgctgatgttgaagacgacaccacggctttgatggaatatcagatattgaaaatgtctctctgcctgttcatccttctgtttctcacacctggtattttatgcatttgtcctctccaatgtatatgcacagagaggcacaggcatgtggactgttcaggcagaaacttgtctacattaccatctggactgcaagagaatattatacatttaaacctgtcttataaccactttactgatctgcataaccagttaacccaatataccaatctgaggaccctggacatttcaaacaacaggcttgaaagcctgcctgctcacttacctcggtctctgtggaacatgtctgctgctaacaacaacattaaacttcttgacaaatctgatactgcttatcagtggaatcttaaatatctggatgtttctaagaacatgctggaaaaggttgtcctcattaaaaatacactaagaagtctcgaggttctcaacctcagtagtaacaaactttggacagttccaaccaacatgccctccaaactacatatcgtggacctgtctaataattctttgacacaaattcttccaggtacattaataaacctgacaaatctcacacatctttacctgcacaacaataagttcacattcattccagaccaatcttttgaccaactctttcagttgcaagagataaccctttacaataacaggtggtcatgtgaccacaaacaaaacattacttacttactgaagtggatgatggaaacaaaagcccatgtgatagggactccatgttctacccaaatatcatctttaaaggaacataacatgtatcccacaccttctggatttacctcaagcttattcactgtaagtgggatgcagacagtggacaccattaactctctgagtgtggtaactcaacccaaagtgaccaaaatacccaaacaatatcgaacaaaggaaacaacgtttggtgccactctaagcaaagacaccacctttactagcactgataaggcttttgtgccctatccagaagatacatccacagagactatcaattcacatgaagcagcagctgcaactctaactattcatctccaagatggaatggtcacaaacacaagcctcactagctcaacaaaatcatccccaacacccatgaccctaagtatcactagtggcatgccaaataatttctctgaaatgcctcaacaaagcacaacccttaacttatggagggaagagacaaccacaaatgtaaagactccattaccttctgtggcaaatgcttggaaagtaaatgcttcatttctcttattgctcaatgttgtggtcatgctggctgtctgagggtctgcattttctgaaactaatgaaagcactcctccctgatgtacagttgggaaaatatgtccatatctaaccagtgattcgagctatatttaagtattcaagaaagccagtcttaacatttctaactctgatgtaaatgaagtaacttgtcttaaataaaagaaatgcacaatgtcttggtacttgctgctattttactgtcttaattaagtaaactaatgagtttcttttataaaaaaaatgaaatgttttaaggcttcaatttattgcacaaaatataaagcatctaaactttaatatgtattttatgtatgtttacactgtcaaacatctggaaaataaaaggtctatgctc

SEQ ID NO: 8 - protein, 433 aa, Homo sapiens [sequence omitted]

SEQ ID NO: 9 - DNA, 1794 bp, Homo sapiens
ctgagctggcaagcagagcccacagccagaaacccttccgactcccacaacaagacgacctttaagctgcaagtttcccggagaaaatgagatactgatagtgaagacgacattatgggctttgatggaatatcagatactgaaaatgtcttcctgcctgttcatccttctgtttctcacgcctggcatcttatgcatttgtcctctccagtgtacatgcacagagaggcacaggcatgtggactgttcaggcagaaacttgactacattaccacctggactgcaggagaacattatacatttaaacctgtcttataaccactttactgatctgcataaccagttaaccccatataccaatctgagaaccctggatatttcaaacaacaggcttgaaagtctgcctgctcagttacctcggtctctctggaacatgtctgctgctaacaacaatattaaacttcttgacaaatctgatactgcttatcagtggaaccttaaatacctggatgtttctaagaatatgctggaaaaggttgttctcattaaaaataccctaagaagtctcgaggttcttaacctcagcagtaacaagctttggacagttccaaccaacatgccttccaaactgcatatcgtggacctgtctaataactcactgacacaaatccttccagggacattaataaacctgacaaatctcacacatctttacctgcacaacaataaattcacattcattccagaacagtcttttgaccaacttttgcagttgcaagagataactcttcataataacaggtggtcatgtgaccataaacaaaacattacttacttattgaagtgggtgatggaaacgaaagcccatgtgatagggactccttgttctaagcaagtatcctctctaaaggaacagagcatgtaccccacacctcctgggtttacctcaagcttatttactatgagtgagatgcagacagtggacaccattaactctttgagtatggtaactcaacccaaagtgaccaaaacacccaaacaatatcgaggaaaggaaaccacatttggtgtcactctaagcaaagataccacttttagtagcactgatagggctgtggtggcctacccagaagacacacccacagaaatgaccaattcccatgaagcagcagctgcaactctaactattcacctccaggatggaatgagttcaaatgcaagcctcaccagtgcaacaaagtcacccccaagccccgtgaccctcagcatagctcgtggcatgccaaataacttctctgaaatgcctcgacaaagcacaaccctcaacttacggagggaagaaaccactgcaaatggaaacactcggccaccttctgcggctagtgcttggaaagtaaatgcctcgctccttttaatgctcaatgctgtggtcatgctggcaggctgagggtctgcagtttctgaaacgaaggagaaccttcctccatgatgtacagttgggaaaacgtgcccctatctaaccagtgattcaagctatattatgtattcaagaaagccagtcttatatttctgactttgatgtaaatgaagtaatttgtcttaattaaaagaagtgcacaatgtcttggtacttgctgctattttcctgtcttaagtaaaactaatgacttttttttttaatgaaatgttttctttttaaggcttcaacttattgcacaaactataaagagcatctaaactttaatatgtattttatgtatgtttacactgtcaaatgtctgggacaaaataaaa

SEQ ID NO: 10 - protein, 440 aa, Homo sapiens
MEYQILKMSSCLFILLFLTPGILCICPLQCTCTERHRHVDCSGRNLTTLPPGLQENIIHLNLSYNHFTDLHNQLTPYTNLRTLDISNNRLESLPAQLPRSLWNMSAANNNIKLLDKSDTAYQWNLKYLDVSKNMLEKVVLIKNTLRSLEVLNLSSNKLWTVPTNMPSKLHIVDLSNNSLTQILPGTLINLTNLTHLYLHNNKFTFIPEQSFDQLLQLQEITLHNNRWSCDHKQNITYLLKWVMETKAHVIGTPCSKQVSSLKEQSMYPTPPGFTSSLFTMSEMQTVDTINSLSMVTQPKVTKTPKQYRGKETTFGVTLSKDTTFSSTDRAVVAYPEDTPTEMTNSHEAAAATLTIHLQDGMSSNASLTSATKSPPSPVTLSIARGMPNNFSEMPRQSTTLNLRREETTANGNTRPPSAASAWKVNASLLLMLNAVVMLAG

SEQ ID NO: 11 - DNA, 22 bp, Artificial sequence
  Synthetic primer used to generate the OMgp probe
tccagggtcctcagattggtat

SEQ ID NO: 12 - DNA, 22 bp, Artificial sequence
  Synthetic primer used to create knock-out mice
ccgaatgctaactgacccatgc

SEQ ID NO: 13 - DNA, 22 bp, Artificial sequence
  Synthetic primer used to create knock-out mice
gaacagtccacatgcctgtgcc

SEQ ID NO: 14 - DNA, 21 bp, Artificial sequence
  Synthetic primer used to create knock-out mice
gatgcccttcagctcgatgcg

SEQ ID NO: 15 - DNA, 20 bp, Artificial sequence
  Synthetic primer used to create OMgp probe
gcccacagccagaaacagtt

SEQ ID NO: 16 - DNA, 384 bp, Artificial sequence
  35-E08 HV
gaagttcaattgttagagtctggtggcggtcttgttcagcctggtggttctttacgtctttcttgcgctgcttccggattcactttctcttattacgatatgggttgggttcgccaagctcctggtaaaggtttggagtgggtttcttggatctctccttctggtggcaagactgtttatgctgactccgttaaaggtcgcttcactatctctagagacaactctaagaatactctctacttgcagatgaacagcttaagggctgaggacacggccgtgtattactgtgcgagagagccaggggaatattgtagtggtggtagctgctacccggactacggtatggacgtctggggccaagggaccacggtcaccgtctcaagc

SEQ ID NO: 17 - DNA, 321 bp, Artificial sequence
  35-E08 LV
gacatccaaatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtgggtctgggacagatttcactctcaccatcagcagtctgcaacctgaagattttgcaacttactactgtcaacagagttacactacccctcgaacgttcggccaagggaccaaggtggacatcaga

SEQ ID NO: 18 - DNA, 360 bp, Artificial sequence
  36-A08 HV
gaagttcaattgttagagtctggtggcggtcttgttcagcctggtggttctttacgtctttcttgcgctgcttccggattcactttctctgcttactggatggattgggttcgccaagctcctggtaaaggtttggagtgggtttctgttatcggtccttctggtggctttactgattatgctgactccgttaaaggtcgcttcactatctctagagacaactctaagaatactctctacttgcagatgaacagcttaagggctgaggacacggccgtgtattactgtgcgaaagatctcatgggatacagctatggttctggctactggggccagggcaccctggtcaccgtctcaagc

SEQ ID NO: 19 - DNA, 330 bp, Artificial sequence
  36-A08 LV
cagagcgaattgactcagcctgcctccgtgtctgggtctcctggacaggcgatcaccatctcctgcactggaaccagcagtgacgttggtggttataactatgtctcctggtaccaacagcacccaggcaaagcccccaaactcatgatttatgaggtcagtaatcggccctcaggggtttcaaatcgcttctctggctccaagtctggcaacacggcctccctgaccatctctgggctccaggctgaggacgaggctgattattactgcagctcatatacaagcagcagcacatgggtgttcggcggagggaccaaactgaccgtcctg

SEQ ID NO: 20 - DNA, 369 bp, Artificial sequence
  36-H06 HV
gaagttcaattgttagagtctggtggcggtcttgttcagcctggtggttctttacgtctttcttgcgctgcttccggattcactttctctctttacgatatggattgggttcgccaagctcctggtaaaggtttggagtgggtttcttctatctcttcttctggtggcggtacttcttatgctgactccgttaaaggtcgcttcactatctctagagacaactctaagaatactctctacttgcagatgaacagcttaagggctgaggacacagccacatattactgtgcgagacgtctggattacgatttttggagggcgggagcttttgatatctggggccaagggacaatggtcaccgtctcaagc

SEQ ID NO: 21 - DNA, 321 bp, Artificial sequence
  36-H06 LV
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcgaacgttcggccaagggaccaaggtggaaatcaaa

SEQ ID NO: 22 - DNA, 366 bp, Artificial sequence
  38-B02 HV
gaagttcaattgttagagtctggtggcggtcttgttcagcctggtggttctttacgtctttcttgcgctgcttccggattcactttctctgtttacgatatgatgtgggttcgccaagctcctggtaaaggtttggagtgggtttcttctatctggtcttctggtggcggtacttattatgctgactccgttaaaggtcgcttcactatctctagagacaactctaagaatactctctacttgcagatgaacagcttaagggctgaggacacagccgtgtattactgtacgagacatgctggtgggagctaccaaaatgatgcttttgatatctggggccaagggacaatggtcaccgtctcaagc

SEQ ID NO: 23 - DNA, 321 bp, Artificial sequence
  38-B02 LV
gacatccagatgacccagtctccatcttccgtgtctgcatctgtgggagacagagtcaccatcacttgtcgggcgagtcaaggtattaacagctggttagcctggtatcagcagaaacctgggacagcccctaaactcctgatctctgctgcatccagtttgcaagatggggtcccatcaaggttcagcggcggtggatctgggacagagttcactctcaccatcagcagcctgcagcctgaagattttgcaacttactattgtcaacagggtaacagtttccctccgaccttcggcggagggaccagagtagagatcaga

SEQ ID NO: 24 - DNA, 372 bp, Artificial sequence
  38-F11 HV
gaagttcaattgttagagtctggtggcggtcttgttcagcctggtggttctttacgtctttcttgcgctgcttccggattcactttctctatttacgctatggagtgggttcgccaagctcctggtaaaggtttggagtgggtttcttctatctctccttctggtggctggactgagtatgctgactccgttaaaggtcgcttcactatctctagagacaactctaagaatactctctacttgcagatgaacagcttaagggctgaggacacggccgtgtattactgtgcgagaaccattaattattactatgatagtagtggttatgtaggggactactggggccagggaaccctggtcaccgtctcaagc

SEQ ID NO: 25 - DNA, 321 bp, Artificial sequence
  38-F11 LV
gacatccagatgacccagtctccttccaccctgtctgcctctgtaggagacagagtcaccatgacttgccgggccagtcagaatattagtacctggttggcctggtatcagcagaaaccagggaaagcccctaaactcctgatctatgaggcatctaatttagaaactggggtcccatcaaggttcagcggcagtggatctgggacagaattcactctcaccatcgccagcctgcagcctgatgattttgcagcttattactgccaacagtatcagacttattcgtggacgttcggccaagggaccaaggtggatgtcaaa

SEQ ID NO: 26 - DNA, 372 bp, Artificial sequence
  38-H02 HV
gaagttcaattgttagagtctggtggcggtcttgttcagcctggtggttctttacgtctttcttgcgctgcttccggattcactttctctttttacgatatggtttgggttcgccaagctcctggtaaaggtttggagtgggtttctcgtatctatccttctggtggccctacttcttatgctgactccgttaaaggtcgcttcactatctctagagacaactctaagaatactctctacttgcagatgaacagcttaagggctgaggacacggccgtgtattactgtgcgagggccagccagtattactatggttcggggagttatgcttttgatatctggggccaagggacaatggtcaccgtctcaagc

SEQ ID NO: 27 - DNA, 321 bp, Artificial sequence
  38-H02 LV
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccttggacgttcggccaagggaccaaggtggaaatcaaa

SEQ ID NO: 28 - DNA, 372 bp, Artificial sequence
  39-F09 HV
gaagttcaattgttagagtctggtggcggtcttgttcagcctggtggttctttacgtctttcttgcgctgcttccggattcactttctctctttacgagatgggttgggttcgccaagctcctggtaaaggtttggagtgggtttctgttatcggtccttctggtggcgagactttttatgctgactccgttaaaggtcgcttcactatctctagagacaactctaagaatactctctacttgcagatgaacagcttaagggctgaggacacggccgtgtattactgtgcgagaggggcgtcctgggggttccataccgaggaatatgcttttgatatctggggccaagggacaatggtcaccgtctcaagc

SEQ ID NO: 29 - DNA, 321 bp, Artificial sequence
  39-F09 LV
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtgccactggatctgggacagacttcactctctccatcagcagtctgcaacctgaagattttgcaacttactactgtcaacagagtttcagttcccctagcacttttggccaggggaccaacctggagatcaaa

SEQ ID NO: 30 - protein, 11 aa, Artificial sequence
  35-E08 LV-CDR1
RASQSISSYLN

SEQ ID NO: 31 - protein, 7 aa, Artificial sequence
  35-E08 LV-CDR2
AASSLQS

SEQ ID NO: 32 - protein, 9 aa, Artificial sequence
  35-E08 LV-CDR3
QQSYTTPRT

SEQ ID NO: 33 - protein, 5 aa, Artificial sequence
  35-E08 HV-CDR1
YYDMG

SEQ ID NO: 34 - protein, 17 aa, Artificial sequence
  35-E08 HV-CDR2
WISPSGGKTVYADSVKG

SEQ ID NO: 35 - protein, 19 aa, Artificial sequence
  35-E08 HV-CDR3
EPGEYCSGGSCYPDYGMDV

SEQ ID NO: 36 - protein, 107 aa, Artificial sequence
  35-E08 LV
DIQMTQSPSSLSASVGDRVTITCRASQSISSYLNWYQQKPGKAPKLLIYAASSLQSGVPSRFSGSGSGTDFTLTISSLQPEDFATYYCQQSYTTPRTFGQGTKVDIR

SEQ ID NO: 37 - protein, 128 aa, Artificial sequence
  35-E08 HV
EVQLLESGGGLVQPGGSLRLSCAASGFTFSYYDMGWVRQAPGKGLEWVSWISPSGGKTVYADSVKGRFTISRDNSKNTLYLQMNSLRAEDTAVYYCAREPGEYCSGGSCYPDYGMDVWGQGTTVTVSS

SEQ ID NO: 38 - protein, 14 aa, Artificial sequence
  36-A08 LV-CDR1
TGTSSDVGGYNYVS

SEQ ID NO: 39 - protein, 7 aa, Artificial sequence
  36-A08 LV-CDR2
EVSNRPS

SEQ ID NO: 40 - protein, 10 aa, Artificial sequence
  36-A08 LV-CDR3
SSYTSSSTWV

SEQ ID NO: 41 - protein, 5 aa, Artificial sequence
  36-A08 HV-CDR1
AYWMD

SEQ ID NO: 42 - protein, 17 aa, Artificial sequence
  36-A08 HV-CDR2
VIGPSGGFTDYADSVKG

SEQ ID NO: 43 - protein, 11 aa, Artificial sequence
  36-A08 HV-CDR3
DLMGYSYGSGY

SEQ ID NO: 44 - protein, 110 aa, Artificial sequence
  36-A08 LV
QSELTQPASVSGSPGQAITISCTGTSSDVGGYNYVSWYQQHPGKAPKLMIYEVSNRPSGVSNRFSGSKSGNTASLTISGLQAEDEADYYCSSYTSSSTWVFGGGTKLTVL

SEQ ID NO: 45 - protein, 120 aa, Artificial sequence
  36-A08 HV
EVQLLESGGGLVQPGGSLRLSCAASGFTFSAYWMDWVRQAPGKGLEWVSVIGPSGGFTDYADSVKGRFTISRDNSKNTLYLQMNSLRAEDTAVYYCAKDLMGYSYGSGYWGQGTLVTVSS

SEQ ID NO: 46 - protein, 11 aa, Artificial sequence
  36-H06 LV-CDR1
RASQSISSYLN

SEQ ID NO: 47 - protein, 7 aa, Artificial sequence
  36-H06 LV-CDR2
AASSLQS

SEQ ID NO: 48 - protein, 9 aa, Artificial sequence
  36-H06 LV-CDR3
QQSYSTPRT

SEQ ID NO: 49 - protein, 5 aa, Artificial sequence
  36-H06 HV-CDR1
LYDMD

SEQ ID NO: 50 - protein, 17 aa, Artificial sequence
  36-H06 HV-CDR2
SISSSGGGTSYADSVKG

SEQ ID NO: 51 - protein, 14 aa, Artificial sequence
  36-H06 HV-CDR3
RLDYDFWRAGAFDI

SEQ ID NO: 52 - protein, 107 aa, Artificial sequence
  36-H06 LV
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 53 - protein, 123 aa, Artificial sequence
  36-H06 HV
EVQLLESGGGLVQPGGSLRLSCAASGFTFSLYDMDWVRQAPGKGLEWVSSISSSGGGTSYADSVKGRFTISRDNSKNTLYLQMNSLRAEDTATYYCARRLDYDFWRAGAFDIWGQGTMVTVSS

SEQ ID NO: 54 - protein, 11 aa, Artificial sequence
  38-B02 LV-CDR1
RASQGINSWLA

SEQ ID NO: 55 - protein, 7 aa, Artificial sequence
  38-B02 LV-CDR2
AASSLQD

SEQ ID NO: 56 - protein, 9 aa, Artificial sequence
  38-B02 LV-CDR3
QQGNSFPPT

SEQ ID NO: 57 - protein, 5 aa, Artificial sequence
  38-B02 HV-CDR1
VYDMM

SEQ ID NO: 58 - protein, 17 aa, Artificial sequence
  38-B02 HV-CDR2
SIWSSGGGTYYADSVKG

SEQ ID NO: 59 - protein, 13 aa, Artificial sequence
  38-B02 HV-CDR3
HAGGSYQNDAFDI

SEQ ID NO: 60 - protein, 107 aa, Artificial sequence
  38-B02 LV
DIQMTQSPSSVSASVGDRVTITCRASQGINSWLAWYQQKPGTAPKLLISAASSLQDGVPSRFSGGGSGTEFTLTISSLQPEDFATYYCQQGNSFPPTFGGGTRVEIR

SEQ ID NO: 61 - protein, 122 aa, Artificial sequence
  38-B02 HV
EVQLLESGGGLVQPGGSLRLSCAASGFTFSVYDMMWVRQAPGKGLEWVSSIWSSGGGTYYADSVKGRFTISRDNSKNTLYLQMNSLRAEDTAVYYCTRHAGGSYQNDAFDIWGQGTMVTVSS

SEQ ID NO: 62 - protein, 11 aa, Artificial sequence
  38-F11 LV-CDR1
RASQNISTWLA

SEQ ID NO: 63 - protein, 7 aa, Artificial sequence
  38-F11 LV-CDR2
EASNLET

SEQ ID NO: 64 - protein, 9 aa, Artificial sequence
  38-F11 LV-CDR3
QQYQTYSWT

SEQ ID NO: 65 - protein, 5 aa, Artificial sequence
  38-F11 HV-CDR1
IYAME

SEQ ID NO: 66 - protein, 17 aa, Artificial sequence
  38-F11 HV-CDR2
SISPSGGWTEYADSVKG

SEQ ID NO: 67 - protein, 15 aa, Artificial sequence
  38-F11 HV-CDR3
TINYYYDSSGYVGDY

SEQ ID NO: 68 - protein, 107 aa, Artificial sequence
  38-F11 LV
DIQMTQSPSTLSASVGDRVTMTCRASQNISTWLAWYQQKPGKAPKLLIYEASNLETGVPSRFSGSGSGTEFTLTIASLQPDDFAAYYCQQYQTYSWTFGQGTKVDVK

SEQ ID NO: 69 - protein, 124 aa, Artificial sequence
  38-F11 HV
EVQLLESGGGLVQPGGSLRLSCAASGFTFSIYAMEWVRQAPGKGLEWVSSISPSGGWTEYADSVKGRFTISRDNSKNTLYLQMNSLRAEDTAVYYCARTINYYYDSSGYVGDYWGQGTLVTVSS

SEQ ID NO: 70 - protein, 11 aa, Artificial sequence
  38-H02 LV-CDR1
RASQSISSYLN

SEQ ID NO: 71 - protein, 7 aa, Artificial sequence
  38-H02 LV-CDR2
AASSLQS

SEQ ID NO: 72 - protein, 9 aa, Artificial sequence
  38-H02 LV-CDR3
QQSYSTPWT

SEQ ID NO: 73 - protein, 5 aa, Artificial sequence
  38-H02 HV-CDR1
FYDMV

SEQ ID NO: 74 - protein, 17 aa, Artificial sequence
  38-H02 HV-CDR2
RIYPSGGPTSYADSVKG

SEQ ID NO: 75 - protein, 15 aa, Artificial sequence
  38-H02 HV-CDR3
ASQYYYGSGSYAFDI

SEQ ID NO: 76 - protein, 107 aa, Artificial sequence
  38-H02 LV
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 77 - protein, 124 aa, Artificial sequence
  38-H02 HV
EVQLLESGGGLVQPGGSLRLSCAASGFTFSFYDMVWVRQAPGKGLEWVSRIYPSGGPTSYADSVKGRFTISRDNSKNTLYLQMNSLRAEDTAVYYCARASQYYYGSGSYAFDIWGQGTMVTVSS

SEQ ID NO: 78 - protein, 14 aa, Artificial sequence
  38-H11 LV-CDR1
TGTSSDVGGYNYVS

SEQ ID NO: 79 - protein, 7 aa, Artificial sequence
  38-H11 LV-CDR-2
DVSNRPS

SEQ ID NO: 80 - protein, 10 aa, Artificial sequence
  38-H11 LV-CDR3
SSYTSSSTYV

SEQ ID NO: 81 - protein, 5 aa, Artificial sequence
  38-H11 HV-CDR1
FYFMD

SEQ ID NO: 82 - protein, 17 aa, Artificial sequence
  38-H11 HV-CDR2
VISPSGGSTFYADSVKG

SEQ ID NO: 83 - protein, 10 aa, Artificial sequence
  38-H11 HV-CDR3
DLVYGDHGGY

SEQ ID NO: 84 - protein, 110 aa, Artificial sequence
  38-H11 LV
QYELTQPASVSGSPGQSITISCTGTSSDVGGYNYVSWYQQHPGKAPKLMIYDVSNRPSGVSNRFSGSKSGNTASLTISGLQAEDEADYYCSSYTSSSTYVFGTGTKVTVL

SEQ ID NO: 85 - protein, 119 aa, Artificial sequence
  38-H11 HV
EVQLLESGGGLVQPGGSLRLSCAASGFTFSFYFMDWVRQAPGKGLEWVSVISPSGGSTFYADSVKGRFTISRDNSKNTLYLQMNSLRAEDTAVYYCARDLVYGDHGGYWGQGTLVTVSS

SEQ ID NO: 86 - protein, 11 aa, Artificial sequence
  39-F09 LV-CDR1
RASQSISSYLN

SEQ ID NO: 87 - protein, 7 aa, Artificial sequence
  39-F09 LV-CDR2
AASSLQS

SEQ ID NO: 88 - protein, 9 aa, Artificial sequence
  39-F09 LV-CDR3
QQSFSSPST

SEQ ID NO: 89 - protein, 5 aa, Artificial sequence
  39-F09 HV-CDR1
LYEMG

SEQ ID NO: 90 - protein, 17 aa, Artificial sequence
  39-F09 HV-CDR2
VIGPSGGETFYADSVKG

SEQ ID NO: 91 - protein, 15 aa, Artificial sequence
  39-F09 HV-CDR3
GASWGFHTEEYAFDI

SEQ ID NO: 92 - protein, 107 aa, Artificial sequence
  39-F09 LV
DIQMTQSPSSLSASVGDRVTITCRASQSISSYLNWYQQKPGKAPKLLIYAASSLQSGVPSRFSATGSGTDFTLSISSLQPEDFATYYCQQSFSSPSTFGQGTNLEIK

SEQ ID NO: 93 - protein, 124 aa, Artificial sequence
  39-F09 HV
EVQLLESGGGLVQPGGSLRLSCAASGFTFSLYEMGWVRQAPGKGLEWVSVIGPSGGETFYADSVKGRFTISRDNSKNTLYLQMNSLRAEDTAVYYCARGASWGFHTEEYAFDIWGQGTMVTVSS